(19) United States
(12) Patent Application Publication
(10) Pub. No.: US 2025/0222598 A1
(43) Pub. Date: Jul. 10, 2025

(54) SYSTEM, METHOD, AND APPARATUS FACILITATING AUTOMATED MANUFACTURE OF CELL THERAPY

(71) Applicant: Multiply Labs Inc., San Francisco, CA (US)

(72) Inventors: Federico PARIETTI, San Francisco, CA (US); Jeffrey Ackerman CURHAN, Warwick, RI (US)

(21) Appl. No.: 19/011,570

(22) Filed: Jan. 6, 2025

Related U.S. Application Data
(60) Provisional application No. 63/698,008, filed on Sep. 23, 2024.
(60) Provisional application No. 63/618,280, filed on Jan. 5, 2024.

(51) Int. Cl. B25J 15/04; F16L 37/00

(52) U.S. Cl. CPC B25J 15/0408; F16L 37/002

(57) ABSTRACT

Disclosed are devices, systems, biological foundries, and methods for facilitating automated modular manufacture of cellular engineering targets.

### CROSS-REFERENCE TO RELATED APPLICATION

The present application claims priority to U.S. Provisional Patent Application No.: 63/618,280 filed Jan. 5, 2024, and U.S. Provisional Patent Application No.: 63/698,008 filed Sep. 23, 2024, each of which is hereby incorporated by reference in its entirety for all purposes.

### TECHNICAL FIELD

The present disclosure generally relates to devices, systems and methods for facilitating automated manufacturing at a biological foundry.

### BACKGROUND

Cell therapies are next-generation drugs where live cells are used to treat a subject. This is in contrast with traditional small-molecule and biologic drugs, where small or large molecules—but not whole living cells—are used to treat patients. Many of the most recent and promising innovations in medicine are represented by cell therapies in which the cells of a subject (either the patient or a donor) are extracted, genetically engineered in a lab, grown in an incubator, and finally infused in the patient in order to achieve a therapeutic effect. However, despite the life-saving effects of many cell therapies, there are significant bottlenecks to their widespread adoption. For instance, one obstacle is represented by the current limits in manufacturing capacity for cell therapies. Conventional cell therapy production processes are still largely labor-based and inefficient.

Traditionally, cell therapies are produced with labor-intensive processes. These conventional processes require not only a large number of manufacturing operators, but also the employment of highly skilled (and expensive) technicians. These constraints make it particularly difficult to manufacture cell therapies at an industrial scale. Cell therapy manufacturing processes are low-scale and labor-intensive because they were originally developed in the context of academic research. The original lab processes—which were developed to demonstrate the feasibility of cell therapies—were then hastily modified and retrofitted in order to fulfill regulatory requirements and achieve good manufacturing practices.

This conventional approach allowed drug manufacturers to bring to the market the first approved cell therapies. However, this labor-intensive, lab-oriented approach is unsuitable to achieve industrial scale. At their core, current cell manufacturing processes were designed to be manually completed by highly trained personnel—such as the researchers that conduct scientific experiments in an academic environment. Requiring this type of skillset becomes a disadvantage in an industrial setting. Cell manufacturing processes depend on highly trained, highly educated manual labor, and this makes them incompatible with the efficiency of mass-manufacturing industrial processes.

The dominant conventional approach to cell manufacturing is based on a set of separate individual pieces of manufacturing equipment placed on a clean room bench. This manufacturing process still looks exactly like a research laboratory, where all the machinery is manually operated and directly supervised by highly skilled operators. In order to execute the cell manufacturing processes, these skilled operators gown up, enter a clean room, and manually activate the machines. The operators also transfer the batch material from machine to machine, manually sample the batches to perform quality control testing, ensure that reagents are delivered to the cells, and ensure that waste material is removed. This labor-based conventional approach is very different from the organization of industrial-scale processes, where most tasks are autonomously executed by specialized machinery, which is supervised by ordinary manufacturing technicians (not engineers, nor scientists).

As such, the conventional labor-based approach to cell therapy manufacturing has at least three fundamental limits. First, the conventional approach is not scalable and not robust to operator variability. Because the conventional approach is extremely labor-intensive, cell therapy manufacturing is limited to small-scale applications. Increasing throughput beyond a few hundred products per year has proven extremely difficult, because such an effort would require hiring, training, retaining, and managing a large number of highly skilled, expensive operators. Moreover, labor-based processes are typically unable to reach industrial scale, and cell manufacturing is not an exception. This pronounced reliance of labor presents additional disadvantages, including the fact that—because of operator variability—the yield and the features of the finished cell therapy product are hard to predict and to control. This operator variability makes scaling the process of manufacturing cell therapy products even harder—particularly in terms of margins, in which a higher number of rejected batches increases the cost per batch.

Additionally, the conventional approach to manufacturing cell therapy products is inefficient. Since individual machines for the cell therapy manufacturing process are utilized in series (e.g., the machines are used one at a time, with a single batch manually moved from a piece of machinery to the next), when a machine is active all the others are idle. This results in a low utilization rate for all machines, since most of the machines are waiting for the batch to arrive, while a single machine is being used. The problem of a very low utilization rate is particularly evident for cell manufacturing processes, which are characterized by machines with markedly different cycle times. More specifically, systems like bioreactors process a single batch for weeks, while machines like thawing and freezing systems are only used for a few hours on a single batch. This results in utilization rates that are even lowed for the faster machines—because the slower machines are the bottleneck and limit the rate of the rest of the serial process.

Finally, the conventional approach to manufacturing cell therapy products has low throughput. Because the process is managed and executed by human operators, only one batch can be produced at any given time on a serial production line. For instance, if two batches were manufactured at the same time on the same production line, in fact, there would be high risk of cross-contamination or of mix-up errors by the operators. Since all the serial machines are used for just one product at a time, the resulting throughput of the production line is extremely low. As a reference, typically a cell therapy product takes two to three weeks to be manufactured. This means that, in order to avoid mix-ups, a whole production line must be reserved for a single product for about half of a month—a rate that is incompatible with industrial scale. Because of this temporal constraint, a whole manufacturing suite (typically consisting of about 1,000 square feet of clean room space) must be reserved for a single serial production line. Therefore, the only way to increase throughput via this conventional approach is by creating facilities with multiple independent suites that replicate the same process. However, each suite can only handle one product at a time, occupies significant clean room space, and is entirely operated by skilled labor. As such, this conventional approach is not scalable, and not suitable to manufacture more than a few hundreds of cell therapies per year—with very high production costs.

One solution to this conventional approach are closed system cell therapy machines that have been developed to attempt to address the shortcomings of the traditional approach. However, even this solution is still labor-intensive and inadequate to reach industrial scale. For instance, this solution can be described as an end-to-end serial system that is contained into a single machine. Different parts of the same machine perform the different steps of the production process. In other words, a single piece of equipment contains all the sub-systems that are needed to perform the cell manufacturing process. An intricate set of tubes connects all of these systems, so that the cell therapy product (which is typically in liquid form) can be transferred from one sub-system to the next without being exposed to the external environment, which provides the closed system.

However, these end-to-end, closed systems are sold as a unique piece of machinery. As such, the machinery cannot be modified by the buyer: once a system is bought, the buyer is constrained to run the exact process for which that machine was designed. Additionally, the machinery still needs to be operated by a highly skilled technician, who needs to perform a complicated set of actions to set up, monitor, and manage the manufacturing process. More specifically, highly trained operators set up the intricate network of tubes that is required by each batch. These operators are also tasked with opening and closing the valves that regulate the flow of material from one part of the system to the next. Furthermore, technicians also manually sample the batch, whenever testing is needed for quality control.

As such, this prior closed system solution suffers disadvantages, in that the closed system solution is overcomplicated. Setting up dozens of tubes, liquid reservoir bags, and reagents requires highly trained labor. This setting up process also takes a long time—even for a skilled technician—to set up, operate, and supervise the machinery. This results in the need for a number of operators that increases proportionally to the number of production system—making it impossible to achieve industrial scale and contain manufacturing costs.

Furthermore, the prior closed system solution is inefficient. Since the architecture of the closed system is still serial, this approach suffers of the same efficiency constraints as the dominant (bench-based) approach. At any given time, most of the subsystems inside of the end-to-end machine are unused. This happens because only one system can be used at a time—this is a serial production line with the hard limit of a single product per production run. Moreover, since some parts of the process are particularly slow (for example, the expansion of the cells into a bioreactor), the subsystems are characterized by an even lower utilization rate than the slower subsystems of the machinery.

Additionally, this closed system lacks design flexibility. This inflexibility draw back is typical of closed systems that are built specifically to execute a particular process. Once the machinery is bought, it is not possible to replace an outdated subsystem with a better one (for example, a subsystem that performs a task better, or with a higher throughput). Any modification to the original closed system machinery requires massive engineering and retooling costs, comparable to building a whole new end-to-end system from scratch. This lack of flexibility is particularly disadvantageous in the case of cell therapy manufacturing—where processes are often tuned and improvement at all stages of clinical development.

Moreover, since each closed system is end-to-end and can only manufacture a single product at a time, the only way to increase throughput is to buy more of these closed systems. This in turn worsens the above-mentioned complexity and underutilization problems. In other words, deploying more complex systems increases the need for skilled operators, which in turn increases the cost of manufacturing. Since each machine is largely underutilized (only one subsystem is active at any given time), chronic underutilization also characterizes a facility that is equipped with multiple end-to-end systems. Furthermore, conventional docking station designs does lend themselves to application in cell therapy manufacturing. For instance, conventional docking stations do not include passive compliance and passive damping systems. Instead, conventional docking stations utilize rigid features, jigs, pins, chamfers, and the like.

Prior solutions, like wedges and chamfers, are easy for robotic systems to interface with. However, wedges and chamfers can only keep a part in place due to gravity. This is inadequate when there are vibrations (i.e., the wedge could move the part outside of the docking station), or when forces perpendicular to gravity could be exerted on the part. For example, if the part is pushed from the side, it can easily slide out of a chamfered docking station. On the other hand, prior solutions like locating pins are hard to operate for robots, because: the locating pins require high accuracy; and the locating pins have a high rigidity, which means that they are not tolerant to misalignments. This affects negatively the repeatability of the process, which would present a higher risk of failure for pick and/or place operations conducting during the manufacture of cell therapies.

Additionally, a major problem of labor-based cell manufacturing processes is that human operators need to sample each batch manually. In cell manufacturing processes, sterility must be always ensured. This is particularly important, because cell therapies cannot be sterilized at the end of the manufacturing process (that would kill the cells). At the same time, guaranteeing the quality of cell manufacturing processes requires a large number of quality control steps. And, in order to perform quality control tests, the cell therapy products must be frequently sampled (i.e. a part of the product must be removed from the batch, while ensuring the sterility of both the sample and the product). In conventional cell manufacturing processes, sampling tasks are executed by human operators.

One disadvantage of this conventional approach to sampling is that human operators are a significant potential source of contamination for cell therapy products. Every time a batch is sampled manually, there is a high risk of contamination because the operator must manually remove a part of the liquid containing the cell product. Even semi-automated sampling procedures, where an operator activates a system that performs the sampling task, present significant risk of contamination due to requiring the presence of a human technicians in close proximity to the process.

Another critical issue is that sampling procedures are performed extremely frequently in cell manufacturing processes. Cell therapy products are sometimes sampled multiple times during a single day. Since cell manufacturing processes have a long completion time (most require more than a week, and many can take up to fifteen to twenty days), manual sampling is repeated dozens of times for every single batch. Repeating risky sampling procedures with this extreme frequency greatly increases the risk of contamination.

Given the above background, there is a need in the art for improved systems, methods, and apparatuses for facilitating an improved manufacture of cell therapies that addresses these dilemmas.

The information disclosed in this background section is only for enhancement of understanding of the general background of the invention and should not be taken as an acknowledgement or any form of suggestion that this information forms the prior art already known to a person skilled in the art.

### SUMMARY

Advantageously, the devices, systems and methods detailed in the present disclosure address the shortcomings in the prior art detailed above.

Systems, methods, and apparatuses for broadly implementing a manufacture of a cellular engineering target at a biological foundry system are provided.

Specifically, exemplary systems, methods, and apparatuses of the present disclosure directly apply to both manufacturing systems for producing small-cellular engineering targets, high-mix cellular engineering targets, personalized cellular engineering targets, or just-in-time production of such targets.

The methods and apparatuses of the present disclosure have other features and advantages which will be apparent from or are set forth in more detail in the accompanying drawings, which are incorporated herein, and the following Detailed Description, which together serve to explain certain principles of the present invention.

### BRIEF DESCRIPTION OF THE DRAWINGS

FIG. 1 illustrates an exemplary biological foundry workflow system topology including a computer system and a plurality of instruments associated with a biological foundry, in accordance with some exemplary embodiments of the present disclosure.

FIG. 2 illustrates various modules and/or components of a computer system, in accordance with some exemplary embodiments of the present disclosure.

FIGS. 3A, 3B, 3C, 3D, and 3E illustrate an exemplary port in accordance with some exemplary embodiments of the present disclosure.

FIGS. 4A, 4B, and 4C illustrate an exemplary coupler in accordance with some exemplary embodiments of the present disclosure.

FIGS. 5A, 5B, and 5C illustrate a first exemplary robotic end of arm tool (EOAT) in accordance with some exemplary embodiments of the present disclosure.

FIGS. 6A, 6B, and 6C illustrate a second exemplary robotic EOAT in accordance with some exemplary embodiments of the present disclosure.

FIGS. 7A-7B illustrate a third exemplary robotic EOAT in accordance with some exemplary embodiments of the present disclosure.

FIGS. 8A, 8B, 8C, 8D, and 8E illustrate a fourth exemplary robotic EOAT in accordance with some exemplary embodiments of the present disclosure.

FIGS. 9A-9D illustrate a device for singulating one or more disinfecting caps (DCaps) in accordance with some exemplary embodiments of the present disclosure.

FIGS. 10A-10B illustrate a first exemplary cartridge in accordance with some exemplary embodiments of the present disclosure.

FIGS. 11A-11B illustrate a first exemplary dock in accordance with some exemplary embodiments of the present disclosure.

FIGS. 12A-12F illustrate a second exemplary cartridge in accordance with some exemplary embodiments of the present disclosure.

FIG. 13 illustrates a second exemplary dock in accordance with some exemplary embodiments of the present disclosure.

FIGS. 14A-14C illustrate an exemplary incubator in accordance with some exemplary embodiments of the present disclosure.

FIGS. 15A-15D illustrate an exemplary cart dock system in accordance with some exemplary embodiments of the present disclosure.

FIG. 16 illustrates a third exemplary cartridge in accordance with some exemplary embodiments of the present disclosure.

FIG. 17 illustrates a fourth exemplary cartridge in accordance with some exemplary embodiments of the present disclosure.

FIGS. 18A-18D illustrate an exemplary filtration system in accordance with some exemplary embodiments of the present disclosure.

FIGS. 19A-19D illustrate a fifth exemplary cartridge in accordance with some exemplary embodiments of the present disclosure.

FIGS. 20A-20D illustrate a third exemplary dock in accordance with some exemplary embodiments of the present disclosure.

FIGS. 21A-21B illustrate a sixth exemplary cartridge in accordance with some exemplary embodiments of the present disclosure.

FIGS. 22A-22Q illustrate an exemplary biological foundry and exemplary operations of the biological foundry in accordance with some exemplary embodiments of the present disclosure.

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

The present disclosure provides systems, methods, and apparatuses for facilitating automated modular manufacture of cellular engineering targets. Exemplary systems, methods, and apparatuses for the manufacturing of cellular engineering targets of the present disclosure includes the advantages of modularity, flexibility, and scalability. Moreover, exemplary systems, methods, and apparatuses of the present disclosure retain the benefits of a conventional closed-system processes, such as providing a sterile clean room environment, without sacrificing the aforementioned advantages. Furthermore, exemplary systems, methods, and apparatus of the present disclosure leverage advanced robotic features and technologies that enables the transformation of cellular engineering target manufacturing from labor-based and low-throughput processes to fully industrialized, high-throughput processes with high scale, efficiency and repeatability. Accordingly, an exemplary modular biological foundry system provided by the present disclosure offers the advantages of increased throughput, in that multiple separate cellular engineering targets can be produced at the same time within the modular biological foundry system.

Reference will now be made in detail to various embodiments of the present disclosure, examples of which are illustrated in the accompanying drawing and described below. While the disclosure will be described in conjunction with exemplary embodiments, it will be understood that the present description is not intended to limit the invention(s) to those exemplary embodiments. On the contrary, the invention(s) is/are intended to cover not only the exemplary embodiments, but also various alternatives, modifications, equivalents and other embodiments, which may be included within the spirit and scope of the present invention as defined by the appended claims.

It will also be understood that, although the terms first, second, etc. may be used herein to describe various elements, these elements should not be limited by these terms. These terms are only used to distinguish one element from another. For example, a first instrument could be termed a second instrument, and, similarly, a second instrument could be termed a first instrument, without departing from the scope of the present disclosure. The first instrument and the second instrument are both instruments, but they are not the same instrument.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and or “comprising,” when used in this specification, specify the presence of stated features, integers, steps, operations, elements, and or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

Furthermore, when a reference number is given an “i^{th}” denotation, the reference number refers to a generic component, set, or embodiment. For instance, an application termed “application i” refers to the i^{th} application in a plurality of applications.

The term “about” or “approximately” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

The foregoing description included example systems, methods, techniques, instruction sequences, and computing machine program products that embody illustrative implementations. For purposes of explanation, numerous specific details are set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions below are not intended to be exhaustive or to limit the implementations to the precise forms disclosed. Many modifications and variations are possible in view of the above teachings. The implementations are chosen and described in order to best explain the principles and their practical applications, to thereby enable others skilled in the art to best utilize the implementations and various implementations with various modifications as are suited to the particular use contemplated.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

An aspect of the present disclosure is directed to providing systems, methods, and apparatuses for facilitating automated modular manufacture of cell therapies.

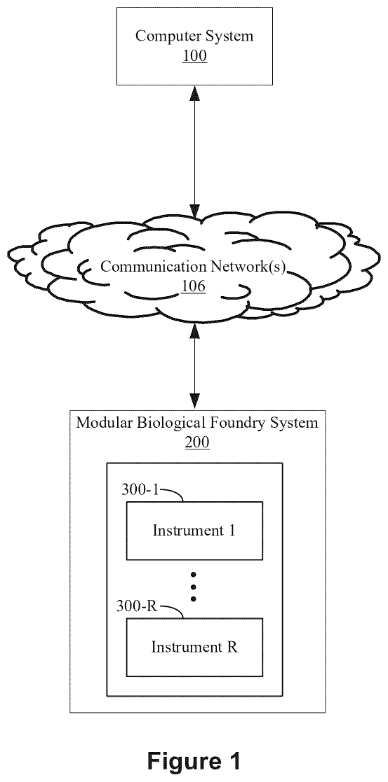

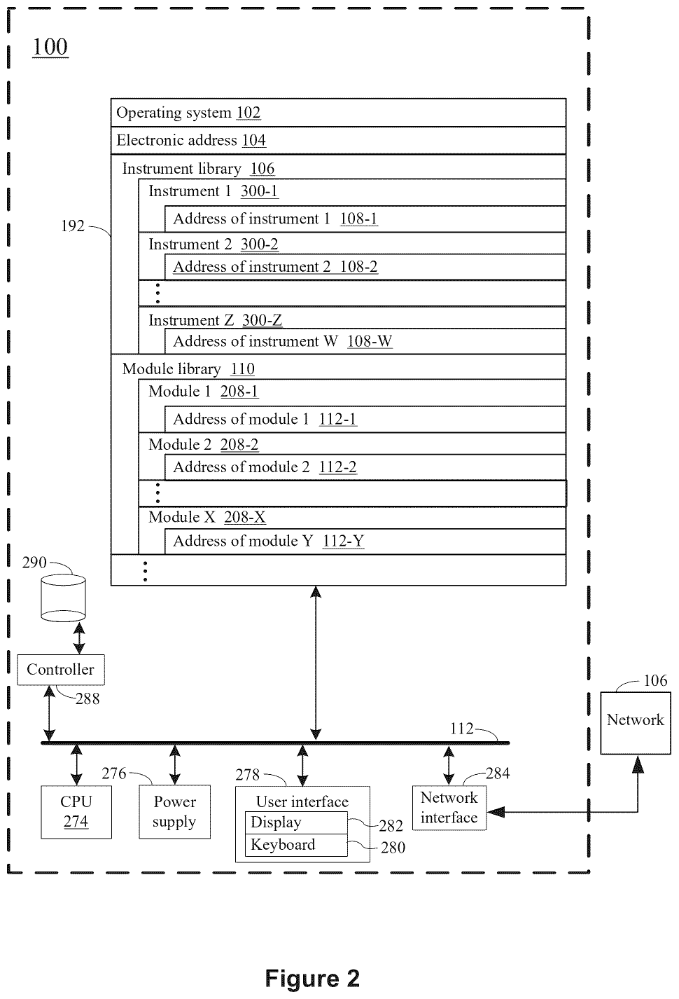

A detailed description of an exemplary system 10 for implementing the automated modular production of cellular engineering targets (e.g., cell therapies) at a biological foundry 200 is described in conjunction with FIG. 1 and FIG. 2. As such, FIG. 1 and FIG. 2 collectively illustrate an exemplary topology of the system 10. In the topology, there is a computer system 100 for generating a workflow that produces a plurality of cellular engineering targets, and providing scheduling of a plurality of instruments in correlation with a corresponding plurality of biological foundry operations, and oversight of the manufacture of the plurality of cellular engineering targets at the modular biological foundry system.

In some embodiments, each cellular engineering target, in the context of biological engineering at a modular biological foundry system, is one of the objectives of a research and development project that defines the desired biological trait to be achieved. The cellular engineering target can be either quantitative or qualitative. For example, in one embodiment, a cellular engineering target(s) can be a genetic configuration for a biosynthetic pathway that produces more compound of interest than a current level. In another embodiment, the cellular engineering target(s) is a genetic configuration for a microbial host that has a tolerance to an inhibitor over X mg/L.

In some embodiments, each cellular engineering target includes modified immune cells or precursors thereof, such as modified T cells, including a chimeric antigen receptor (CAR). Thus, in some embodiments, the immune cell is genetically modified at a modular biological foundry system to express the CAR. In some embodiments, CARs include an antigen binding domain, a transmembrane domain, a hinge domain, and an intracellular signaling domain.

In some embodiments, the antigen binding domain is operably linked to another domain of the CAR, such as the transmembrane domain or the intracellular domain, for expression in the cellular engineering target. In some embodiments, a first nucleic acid sequence encoding the antigen binding domain is operably linked to a second nucleic acid encoding a transmembrane domain, and further operably linked to a third a nucleic acid sequence encoding an intracellular domain.

The antigen binding domains described herein can be combined with any of the transmembrane domains, any of the intracellular domains or cytoplasmic domains, or any of the other domains that may be included in a CAR. In some embodiments, a cellular engineering target CAR of the present disclosure includes a spacer domain. In some embodiments, each of the antigen binding domain, transmembrane domain, and intracellular domain is separated by a linker.

In the present disclosure, the cellular engineering targets generally include mammalian cells, and typically include human cells. In some embodiments, the cellular engineering target is derived from the blood, bone marrow, lymph, or lymphoid organs. In some embodiments, the cellular engineering targets includes cells of the immune system, such as cells of innate or adaptive immunity, e.g., myeloid or lymphoid cells, including lymphocytes, typically T cells and/or NK cells. In some embodiments, the cellular engineering targets include stem cells, such as multipotent and pluripotent stem cells, including induced pluripotent stem cells (iPSCs). The cellular engineering targets typically are primary cells, such as those isolated directly from a subject and/or isolated from a subject and frozen. In some embodiments, the cellular engineering targets include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, degree of differentiation, or a combination thereof. With reference to the subject to be treated, the cellular engineering targets be allogeneic and/or autologous. In some embodiments, the modular biological foundry system facilitates manufacturing the cellular engineering targets by isolating cells from the subject, preparing the cells, processing the cells, culturing the cells, engineering the cells, and re-introducing the cells into the same subject, before or after cryopreservation. In some embodiments, one or more of these steps is performed at modules 208 of modular biological foundry system 200 by utilizing rigid cartridge 1100 and a robot material transfer system.

In some embodiments, among the sub-types and subpopulations of T cells and/or of CD4+ and/or of CD8+ T cells of the cellular engineering targets are naive T (T.sub.N) cells, effector T cells (T.sub.EFF), memory T cells and sub-types thereof, such as stem cell memory T (T.sub.SCM), central memory T (T.sub.CM), effector memory T (T.sub.EM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cellular engineering targets are natural killer (NK) cells. In some embodiments, the cellular engineering targets are monocytes or granulocytes, e.g., myeloid cells, macrophages, neutrophils, dendritic cells, mast cells, eosinophils, and/or basophils.

Accordingly, the present disclosure provides systems and methods for producing or generating a cellular engineering target that is a modified immune cell or precursor thereof (e.g., a T cell) of the invention for tumor immunotherapy, e.g., adoptive immunotherapy. The cellular engineering targets generally are engineered by introducing one or more nucleic acids encoding a subject CAR, dominant negative receptor and/or switch receptor, and/or bispecific antibody, and/or combinations thereof.

In some embodiments, one or more nucleic acids encoding the subject CAR, dominant negative receptor and/or switch receptor, and/or bispecific antibody is introduced into a cell by an expression vector. Expression vectors including a nucleic acid sequence encoding a subject CAR, dominant negative receptor and/or switch receptor, and/or bispecific antibody, and/or combinations thereof, of the present disclosure are provided herein. Suitable expression vectors include lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, Piggybak, and Integrases such as Phi31. Some other suitable expression vectors include Herpes simplex virus (HSV) and retrovirus expression vectors.

Adenovirus expression vectors are based on adenoviruses, which have a low capacity for integration into genomic DNA but a high efficiency for transfecting host cells. Adenovirus expression vectors contain adenovirus sequences sufficient to: (a) support packaging of the expression vector and (b) to ultimately express the subject CAR, dominant negative receptor and/or switch receptor, and/or bispecific antibody, and/or combinations thereof, in the host cell. In some embodiments, the adenovirus genome is a 36 kb, linear, double stranded DNA, where a foreign DNA sequence (e.g., a nucleic acid encoding a subject CAR, dominant negative receptor and/or switch receptor, and/or bispecific antibody, and/or combinations thereof) may be inserted to substitute large pieces of adenoviral DNA in order to make the expression vector of the present invention. Additional details and information can be found at Danthinne et al., 2002, Gene Therapy, 7(20, pg. 1707, which is hereby incorporated by reference in its entirety.

Another expression vector is based on an adeno associated virus, which takes advantage of the adenovirus coupled systems. This AAV expression vector has a high frequency of integration into the host genome. Moreover, this AAV expression can infect non-dividing cells, thus making it useful for delivery of genes into mammalian cells, for example, in tissue cultures or in vivo. The AAV vector has a broad host range for infectivity.

Retrovirus expression vectors are capable of integrating into the host genome, delivering a large amount of foreign genetic material, infecting a broad spectrum of species and cell types and being packaged in special cell lines. The retrovirus vector is constructed by inserting a nucleic acid (e.g., a nucleic acid encoding a subject CAR, dominant negative receptor and/or switch receptor, and/or bispecific antibody, and/or combinations thereof) into the viral genome at certain locations to produce a virus that is replication defective. Though the retrovirus vectors are able to infect a broad variety of cell types, integration and stable expression of the subject CAR, dominant negative receptor and/or switch receptor, and/or bispecific antibody, and/or combinations thereof, requires the division of host cells.

Lentivirus vectors are derived from lentiviruses, which are complex retroviruses that, in addition to the common retroviral genes gag, pol, and env, contain other genes with regulatory or structural function (see, e.g., U.S. Pat. Nos. 6,013,516 and 5,994,136). Some examples of lentiviruses include the Human Immunodeficiency Viruses (HIV-1, HIV-2) and the Simian Immunodeficiency Virus (SIV). Lentivirus vectors have been generated by multiply attenuating the HIV virulence genes, for example, the genes env, vif, vpr, vpu and nef are deleted making the vector biologically safe. Lentivirus vectors are capable of infecting non-dividing cells and can be used for both in vivo and ex vivo gene transfer and expression, e.g., of a nucleic acid encoding a subject CAR, dominant negative receptor and/or switch receptor, and/or bispecific antibody, and/or combinations thereof.

Expression vectors including a nucleic acid of the present disclosure can be introduced into a host cell by any means known to persons skilled in the art. The expression vectors may include viral sequences for transfection, if desired. Alternatively, the expression vectors may be introduced by fusion, electroporation, biolistic, transfection, lipofection, or the like. The host cell may be grown and expanded in culture before introduction of the expression vectors, followed by the appropriate treatment for introduction and integration of the vectors. The host cells are then expanded and may be screened by virtue of a marker present in the vectors. Various markers that may be used are known in the art, and may include hprt, neomycin resistance, thymidine kinase, hygromycin resistance, etc. As used herein, the terms “cell,” “cell line,” and “cell culture” may be used interchangeably. In some embodiments, the host cell an immune cell or precursor thereof, e.g., a T cell, an NK cell, or an NKT cell. In some embodiments, one or more of these steps is performed at modules 208 of modular biological foundry system 200 by utilizing rigid cartridge 1108 and a robot material transfer system.

The present invention also provides genetically engineered cells which include and stably express a subject CAR, dominant negative receptor and/or switch receptor, and/or bispecific antibody, and/or combinations thereof, of the present disclosure. In some embodiments, the genetically engineered cells are genetically engineered T-lymphocytes (T cells), naive T cells (TN), memory T cells (for example, central memory T cells (TCM), effector memory cells (TEM)), natural killer cells (NK cells), and macrophages capable of giving rise to therapeutically relevant progeny. In one embodiment, the genetically engineered cells are autologous cells.

In some embodiments, modified cells (e.g., including a subject CAR, dominant negative receptor and/or switch receptor, and/or expresses and secretes a bispecific antibody, and/or combinations thereof) is produced by stably transfecting host cells with an expression vector including a nucleic acid of the present disclosure. Additional methods to generate a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). Transfected cells expressing a subject CAR, dominant negative receptor and/or switch receptor, and/or bispecific antibody, and/or combinations thereof, of the present disclosure may be expanded ex vivo.

Physical methods for introducing an expression vector into host cells include calcium phosphate precipitation, lipofection, particle bombardment, microinjection, electroporation, and the like. Methods for producing cells including vectors and/or exogenous nucleic acids are well-known in the art. See, Sambrook et al. 2001, “Molecular Cloning: A Laboratory Manual,” Cold Spring Harbor Laboratory, print, which is hereby incorporated by reference in its entirety. Chemical methods for introducing an expression vector into a host cell include colloidal dispersion systems, such as macromolecule complexes, nanocapsules, microspheres, beads, and lipid-based systems including oil-in-water emulsions, micelles, mixed micelles, and liposomes.

In some embodiments, lipids suitable for use in the manufacture of a cellular engineering target at a modular biological foundry system is obtained from commercial sources. Stock solutions of lipids in chloroform or chloroform/methanol can be stored at about −20 degrees C. Chloroform may be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Compositions that have different structures in solution than the normal vesicular structure are also encompassed. For example, the lipids may assume a micellar structure or merely exist as non-uniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the nucleic acids in the host cell, a variety of assays may be performed. Such assays include, for example, molecular biology assays well known to those of skill in the art, such as Southern and Northern blotting, RT-PCR and PCR; biochemistry assays, such as detecting the presence or absence of a particular peptide, e.g., by immunological means (ELISAs and Western blots) or by assays described herein to identify agents falling within the scope of the invention. In some embodiments, one or more of these assays is performed at modules 208 of modular biological foundry system 200 by utilizing rigid cartridge 1100 and a robot material transfer system.

In one embodiment, the nucleic acids introduced into the host cell are RNA. In another embodiment, the RNA is mRNA that comprises in vitro transcribed RNA or synthetic RNA. The RNA may be produced by in vitro transcription using a polymerase chain reaction (PCR)-generated template. DNA of interest from any source can be directly converted by PCR into a template for in vitro mRNA synthesis using appropriate primers and RNA polymerase. The source of the DNA may be, for example, genomic DNA, plasmid DNA, phage DNA, cDNA, synthetic DNA sequence or any other appropriate source of DNA.

PCR may be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers may also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

In some embodiments, chemical structures that have the ability to promote stability and/or translation efficiency of the RNA are used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

The polyA/T segment of the transcriptional DNA template can be produced during PCR by using a reverse primer containing a polyT tail, such as 100 T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art.

In some embodiments, the RNA is electroporated into the cells, such as in vitro transcribed RNA. Any solutes suitable for cell electroporation, which can contain factors facilitating cellular permeability and viability such as sugars, peptides, lipids, proteins, antioxidants, and surfactants can be included.

In some embodiments, a nucleic acid encoding a subject CAR, dominant negative receptor and/or switch receptor, and/or bispecific antibody, and/or combinations thereof, of the present disclosure will be RNA, e.g., in vitro synthesized RNA. Methods for in vitro synthesis of RNA are known in the art; any known method can be used to synthesize RNA comprising a sequence encoding a subject CAR, dominant negative receptor and/or switch receptor, and/or bispecific antibody, and/or combinations thereof. Methods for introducing RNA into a host cell are known in the art. Introducing RNA comprising a nucleotide sequence encoding a subject CAR, dominant negative receptor and/or switch receptor, and/or bispecific antibody, and/or combinations thereof, into a host cell can be carried out in vitro or ex vivo or in vivo. For example, a host cell (e.g., an NK cell, a cytotoxic T lymphocyte, etc.) can be electroporated in vitro or ex vivo with RNA comprising a nucleotide sequence encoding a subject CAR, dominant negative receptor and/or switch receptor, and/or bispecific antibody, and/or combinations thereof.

The disclosed methods can be applied to the modulation of T cell activity in basic research and therapy, in the fields of cancer, stem cells, acute and chronic infections, and autoimmune diseases, including the assessment of the ability of the genetically modified T cell to kill a target cancer cell.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains.

One advantage of RNA transfection methods of the invention is that RNA transfection is essentially transient and a vector-free. A RNA transgene can be delivered to a lymphocyte and expressed therein following a brief in vitro cell activation, as a minimal expressing cassette without the need for any additional viral sequences. Under these conditions, integration of the transgene into the host cell genome is unlikely. Cloning of cells is not necessary because of the efficiency of transfection of the RNA and its ability to uniformly modify the entire lymphocyte population.

Genetic modification of T cells with in vitro-transcribed RNA (IVT-RNA) makes use of two different strategies both of which have been successively tested in various animal models. Cells are transfected with in vitro-transcribed RNA by means of lipofection or electroporation. It is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

In another aspect, the RNA construct is delivered into the cells by electroporation. The various parameters including electric field strength required for electroporation of any known cell type are generally known in the relevant research literature as well as numerous patents and applications in the field. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

In some embodiments, the immune cells (e.g. T cells) can be incubated or cultivated prior to, during and/or subsequent to introducing the nucleic acid molecule encoding the subject CAR, dominant negative receptor and/or switch receptor, and/or bispecific antibody, and/or combinations thereof. In some embodiments, the cells (e.g. T cells) can be incubated or cultivated prior to, during or subsequent to the introduction of the nucleic acid molecule encoding the subject CAR, dominant negative receptor and/or switch receptor, and/or bispecific antibody, and/or combinations thereof, such as prior to, during or subsequent to the transduction of the cells with a viral vector (e.g. lentiviral vector) encoding the subject CAR, dominant negative receptor and/or switch receptor, and/or bispecific antibody, and/or combinations thereof. In some embodiments, the method includes activating or stimulating cells with a stimulating or activating agent (e.g. anti-CD3/anti-CD28 antibodies) prior to introducing the nucleic acid molecule encoding the subject CAR, dominant negative receptor and/or switch receptor, and/or bispecific antibody, and/or combinations thereof. In some embodiments, one or more of these steps is performed at modules 208 of modular biological foundry system 200 by utilizing rigid cartridge 1100 and a robot material transfer system.

In some embodiments, where the nucleic acid sequences encoding the subject CAR, dominant negative receptor and/or switch receptor, and/or bispecific antibody, and/or combinations thereof, of the present invention reside on one or more separate nucleic acid sequences, the order of introducing each of the one or more nucleic acid sequences may vary. For example, a nucleic acid sequence encoding a subject CAR and dominant negative receptor and/or switch receptor may first be introduced into the host cell, followed by introduction of a nucleic acid sequence encoding a subject bispecific antibody. For example, a nucleic acid sequence encoding a subject bispecific antibody may first be introduced into the host cell, followed by introduction of a nucleic acid sequence encoding a subject CAR and dominant negative receptor and/or switch receptor. In some embodiments, each of the one or more nucleic acid sequences are introduced into the host cell simultaneously. Those of skill in the art will be able to determine the order in which each of the one or more nucleic acid sequences are introduced into the host cell.

Prior to expansion, a source of immune cells is obtained from a subject for ex vivo manipulation. Sources of target cells for ex vivo manipulation may also include, e.g., autologous or heterologous donor blood, cord blood, or bone marrow. For example, the source of immune cells may be from the subject to be treated with the modified immune cells of the invention, e.g., the subject's blood, the subject's cord blood, or the subject's bone marrow. Non-limiting examples of subjects include humans, dogs, cats, mice, rats, and transgenic species thereof. Preferably, the subject is a human.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some embodiments, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell a hematopoietic stem cell, a natural killer cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MATT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some embodiments, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components. In some embodiments, one or more of these steps is performed at modules 208 of modular biological foundry system 200 by utilizing rigid cartridge 1100 and a robot material transfer system.

In some examples, cells from the circulating blood of a subject are obtained, e.g., by apheresis or leukapheresis. The samples, in some embodiments, contain lymphocytes, including T cells, monocytes, granulocytes, B cells, other nucleated white blood cells, red blood cells, and/or platelets, and, in some embodiments, contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, a washing step is accomplished by tangential flow filtration (TFF) according to the manufacturer's instructions. In some embodiments, the cells are resuspended in a variety of biocompatible buffers after washing. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient. In some embodiments, one or more of these steps is performed at modules 208 of modular biological foundry system 200 by utilizing rigid cartridge 1100 and a robot material transfer system.

In one embodiment, immune cells are obtained cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample are removed and the cells directly resuspended in culture media. In some embodiments, one or more of these steps is performed at modules 208 of modular biological foundry system 200 by utilizing rigid cartridge 1100 and a robot material transfer system.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. In some embodiments, one or more of these steps is performed at modules 208 of modular biological foundry system 200 by utilizing rigid cartridge 1100 and a robot material transfer system.

Such separation steps can be based on positive selection, in which the cells having bound the reagents are retained for further use, and/or negative selection, in which the cells having not bound to the antibody or binding partner are retained. In some examples, both fractions are retained for further use. In some embodiments, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker.sup.high) of one or more particular markers, such as surface markers, or that are negative for (marker.sup.−) or express relatively low levels (marker.sup.low) of one or more markers. For example, in some embodiments, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD 127, and/or CD62L and/or depleted of (e.g., negatively selected for) cells that are positive for or express high surface levels of CD45RA. In some embodiments, cells are enriched for or depleted of cells positive or expressing high surface levels of CD 122, CD95, CD25, CD27, and/or IL7-Ra (CD 127). In some examples, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD14. In some embodiments, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which, in some embodiments, is particularly robust in such sub-populations. In some embodiments, combining TCM-enriched CD8+ T cells and CD4+ T cells further enhances efficacy.

In embodiments, memory T cells are present in both CD62L+ and CD62L− subsets of CD8+ peripheral blood lymphocytes. PBMC can be enriched for or depleted of CD62L− CD8+ and/or CD62L+CD8+ fractions, such as using anti-CD8 and anti-CD62L antibodies. In some embodiments, a CD4+ T cell population and a CD8+ T cell sub-population, e.g., a sub-population enriched for central memory (TCM) cells. In some embodiments, the enrichment for central memory T (TCM) cells is based on positive or high surface expression of CD45RO, CD62L, CCR7, CD28, CD3, and/or CD 127; in some embodiments, it is based on negative selection for cells expressing or highly expressing CD45RA and/or granzyme B. In some embodiments, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections, in some embodiments, are carried out simultaneously and in other aspects are carried out sequentially, in either order. In some embodiments, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

CD4+ T helper cells are sorted into naive, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO. In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In some embodiments, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL. In some embodiments, one or more of these steps is performed at modules 208 of modular biological foundry system 200 by utilizing rigid cartridge 1100 and a robot material transfer system.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques. In some embodiments, one or more of these steps is performed at modules 208 of modular biological foundry system 200 by utilizing rigid cartridge 1100 and a robot material transfer system.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immuno-adherence or flow cytometry that uses a cocktail of monoclonal antibodies directed to cell surface markers present on the cells negatively selected. For example, to enrich for CD4.sup.+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. In some embodiments, one or more of these steps is performed at modules 208 of modular biological foundry system 200 by utilizing rigid cartridge 1100 and a robot material transfer system.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80 degrees C. at a rate of 1 degrees C. per minute and stored in the vapor phase of a liquid nitrogen storage tank. Other methods of controlled freezing may be used as well as uncontrolled freezing immediately at −20 degrees C. or in liquid nitrogen. In some embodiments, one or more of these steps is performed at modules 208 of modular biological foundry system 200 by utilizing rigid cartridge 1100 and a robot material transfer system.

In one embodiment, the population of T cells includes cells such as peripheral blood mononuclear cells, cord blood cells, a purified population of T cells, and a T cell line. In another embodiment, peripheral blood mononuclear cells include the population of T cells. In yet another embodiment, purified T cells include the population of T cells.

In certain embodiments, T regulatory cells (Tregs) is isolated from a sample. The sample can include, but is not limited to, umbilical cord blood or peripheral blood. In certain embodiments, the Tregs are isolated by flow-cytometry sorting. The sample can be enriched for Tregs prior to isolation by any means known in the art. The isolated Tregs can be cryopreserved, and/or expanded prior to use. In some embodiments, one or more of these steps is performed at modules 208 of modular biological foundry system 200 by utilizing rigid cartridge 1100 and a robot material transfer system.

Whether prior to or after modification of cells to express a subject CAR, dominant negative receptor, and/or switch receptor, and/or bispecific antibody, and/or combinations thereof, the cells can be activated and expanded in number using methods known to one of skill in the art. For example, the T cells of the invention may be expanded by contact with a surface having attached thereto an agent that stimulates a CD3/TCR complex associated signal and a ligand that stimulates a co-stimulatory molecule on the surface of the T cells. In particular, T cell populations may be stimulated by contact with an anti-CD3 antibody, or an antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, T cells can be contacted with an anti-CD3 antibody and an anti-CD28 antibody, under conditions appropriate for stimulating proliferation of the T cells. Examples of an anti-CD28 antibody include 9.3, B-T3, XR-CD28 and these can be used in the present disclosure as can other methods and reagents known in the art.

Expanding T cells by the methods disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the T cells expand in the range of about 20-fold to about 50-fold.

Following culturing, the T cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The T cell medium may be replaced during the culture of the T cells at any time. Preferably, the T cell medium is replaced about every 2 to 3 days. The T cells are then harvested from the culture apparatus whereupon the T cells can be used immediately or cryopreserved to be stored for use at a later time. In one embodiment, the invention includes cryopreserving the expanded T cells. The cryopreserved T cells are thawed prior to introducing nucleic acids into the T cell.

In another embodiment, the method comprises isolating T cells and expanding the T cells. In another embodiment, the invention further comprises cryopreserving the T cells prior to expansion. In yet another embodiment, the cryopreserved T cells are thawed for electroporation with the RNA encoding the chimeric membrane protein. In some embodiments, one or more of these steps is performed at modules 208 of modular biological foundry system 200 by utilizing rigid cartridge 1100 and a robot material transfer system.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time. In some embodiments, one or more of these steps is performed at modules 208 of modular biological foundry system 200 by utilizing rigid cartridge 1100 and a robot material transfer system.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging. Therefore, the number of population doublings of a culture is greater than the passage number. The expansion of cells (i.e., the number of population doublings) during the period between passaging depends on many factors, including but is not limited to the seeding density, substrate, medium, and time between passaging. In some embodiments, one or more of these steps is performed at modules 208 of modular biological foundry system 200 by utilizing rigid cartridge 1100 and a robot material transfer system.

In one embodiment, the cells may be cultured for several hours (about 3 hours) to about 14 days or any hourly integer value in between. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-.alpha.. or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, .alpha.-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37 degrees C.) and atmosphere (e.g., air plus 5% CO.sub.2).

The medium used to culture the T cells may include an agent that can co-stimulate the T cells. For example, an agent that can stimulate CD3 is an antibody to CD3, and an agent that can stimulate CD28 is an antibody to CD28. A cell isolated by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the T cells expand in the range of about 20-fold to about 50-fold, or more. In one embodiment, human T regulatory cells are expanded via anti-CD3 antibody coated KT64.86 artificial antigen presenting cells (aAPCs). In one embodiment, human T regulatory cells are expanded via anti-CD3 antibody coated K562 artificial antigen presenting cells (aAPCs). In some embodiments, one or more of these steps is performed at modules 208 of modular biological foundry system 200 by utilizing rigid cartridge 1100 and a robot material transfer system.

In one embodiment, the method of expanding the T cells can further comprise isolating the expanded T cells for further applications. In another embodiment, the method of expanding can further comprise a subsequent electroporation of the expanded T cells followed by culturing. The subsequent electroporation may include introducing a nucleic acid encoding an agent, such as transducing the expanded T cells, transfecting the expanded T cells, or electroporating the expanded T cells with a nucleic acid, into the expanded population of T cells, wherein the agent further stimulates the T cell. The agent may stimulate the T cells, such as by stimulating further expansion, effector function, or another T cell function. In some embodiments, one or more of these steps is performed at modules 208 of modular biological foundry system 200 by utilizing rigid cartridge 1100 and a robot material transfer system.

### EXAMPLE 1

### Methods of Treatment of a Subject Using Cellular Engineering Targets

In some embodiments, the cellular engineering targets is modified cells (e.g., T cells). In some embodiments, a composition for immunotherapy includes the modified cells. In some embodiments, the composition includes a pharmaceutical composition and further include a pharmaceutically acceptable carrier. In some embodiments, a therapeutically effective amount of the pharmaceutical composition include the modified T cells is administered.

In one aspect, the present disclosure includes a method for adoptive cell transfer therapy including administering to a subject in need thereof a cellular engineering target including a modified T cell of the present disclosure. In another aspect, the present disclosure includes a method of treating a disease or condition in a subject including administering to a subject in need thereof a population of modified T cells

In some embodiments, a method of treating a disease or condition in a subject in need thereof includes administering to the subject a modified cell (e.g., modified T cell) of the present invention. In one embodiment, the method of treating a disease or condition in a subject in need thereof comprises administering to the subject a modified cell (e.g., a modified T cell) comprising a subject CAR, dominant negative receptor and/or switch receptor, and/or a bispecific antibody, and/or combinations thereof. In one embodiment, the method of treating a disease or condition in a subject in need thereof comprises administering to the subject a modified cell (e.g., a modified T cell) comprising a subject CAR (e.g., a CAR having affinity for PSMA on a target cell) and a dominant negative receptor and/or switch receptor. In one embodiment, the method of treating a disease or condition in a subject in need thereof comprises administering to the subject a modified cell (e.g., a modified T cell) comprising a subject CAR (e.g., a CAR having affinity for PSMA on a target cell), a dominant negative receptor and/or switch receptor, and wherein the modified cell is capable of expressing and secreting a bispecific antibody.

Methods for administration of immune cells for adoptive cell therapy are known and may be used in connection with the provided methods and compositions. In some embodiments, autologous transfer conducts the cell therapy, e.g., adoptive T cell therapy, in which the cells are isolated and/or otherwise prepared from the subject who is to receive the cell therapy, or from a sample derived from such a subject. Thus, in some embodiments, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, by isolating and/or otherwise preparing the cells from a subject other than a subject who is to receive or who ultimately receives the cell therapy, e.g., a first subject. In such embodiments, the method includes administering to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the method includes treating the subject with a therapeutic agent targeting the disease or condition, e.g., the tumor, prior to administering of the cells or composition containing the cells. In some embodiments, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administering the cellular engineering target effectively treats the subject despite the subject having become resistant to another therapy.

In some embodiments, the subject is responsive to the other therapeutic agent, and treatment with the therapeutic agent reduces disease burden. In some embodiments, the subject is initially responsive to the therapeutic agent, but exhibits a relapse of the disease or condition over time. In some embodiments, the subject has not relapsed. In some such embodiments, a determination that the subject is at risk for relapse is provided, such as at a high risk of relapse, and thus the method includes administering cellular engineering target prophylactically, e.g., to reduce the likelihood of or prevent relapse. In some embodiments, the subject has not received prior treatment with another therapeutic agent.

In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administrating the cellular engineering target effectively treats the subject despite the subject having become resistant to another therapy.

In some embodiments, the method includes administering the modified immune cells of the cellular engineering target of the present disclosure to an animal, preferably a mammal, even more preferably a human, to treat a cancer. In addition, in some embodiments, the cellular engineering target of the present invention is utilized for the treatment of any condition related to a cancer, especially a cell-mediated immune response against a tumor cell(s), where it is desirable to treat or alleviate the disease. The types of cancers with the modified cells or pharmaceutical compositions of the invention include, carcinoma, blastoma, and sarcoma, and certain leukemia or lymphoid malignancies, benign and malignant tumors, and malignancies e.g., sarcomas, carcinomas, and melanomas. Other exemplary cancers include but are not limited breast cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, and the like. The cancers may be non-solid tumors (such as hematological tumors) or solid tumors. Adult tumors/cancers and pediatric tumors/cancers are also included.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

In some embodiments, carcinomas amenable to therapy by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

In some embodiments, sarcomas amenable to therapy by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Prostate adenocarcinoma is an extremely common and lethal disease. Prostate cancer is the most common malignancy among men. Prostate cancer is the second-leading cause of cancer-related deaths among men, accounting for an estimated 10% of annual male cancer deaths. PSMA is highly expressed in malignant prostate tissue, with low-levels of expression in some normal human tissues. Under normal physiologic conditions, PSMA is expressed in the prostate gland (secretory acinar epithelium), kidney (proximal tubules), nervous system glia (astrocytes and Schwann cells), and the small intestine (jejunal brush border). PSMA is much more highly expressed in prostate epithelium and is significantly upregulated in malignant prostate tissues. PSMA expression in normal cells has been found to be 100-fold to 1000-fold less than in prostate carcinoma cells. PSMA expression increases significantly during the transformation from benign prostatic hyperplasia to prostatic adenocarcinoma. PSMA expression has been found to be directly correlated with the histologic grade of malignant prostate tissue and increases with more advanced disease (i.e. highest PSMA expression found in prostate cancer metastases in lymph node and bone).

In one embodiment, the methods of the invention are useful for treating prostate cancer, for example advanced castrate-resistant prostate cancer. It should be readily understood by one of ordinary skill in the art that any type of cancer wherein the PSMA tumor antigen is expressed, can be treated using the methods of the present invention. For example, neovascular expression of PSMA was found in non-small cell lung cancer. Accordingly, the methods of the invention may also be useful for treating non-small cell lung cancer (NSCLC).

In certain exemplary embodiments, the modified immune cells of the invention treat prostate cancer. In one embodiment, a method of the present disclosure provides a treatment for castrate-resistant prostate cancer. In one embodiment, a method of the present disclosure provides a treatment for advanced castrate-resistant prostate cancer. In one embodiment, a method of the present disclosure provides a treatment for metastatic castrate-resistant prostate cancer. In one embodiment, a method of the present disclosure provides a treatment for metastatic castrate-resistant prostate cancer, wherein the patient with metastatic castrate-resistant prostate cancer has .gtoreq.10% tumor cells expressing PSMA. In one embodiment, a method of the present disclosure provides a treatment for castrate-resistant prostate adenocarcinoma, wherein the patient has castrate levels of testosterone (e.g., <50 ng/ml) with or without the use of androgen deprivation therapy.

In certain embodiments, the method includes providing the subject with a secondary treatment. Secondary treatments include but are not limited to chemotherapy, radiation, surgery, and medications.

In some embodiments, the method includes administering the cellular engineering in dosages and routes and at times determined based on appropriate pre-clinical and clinical experimentation and trials. In some embodiments, the method includes administering cellular engineering target compositions multiple times at dosages within these ranges. The administrating of the cells of the invention includes other methods useful to treat the desired disease or condition as determined by those of skill in the art.

In some embodiments, administrating of the cellular engineering target of the present disclosure includes any convenient manner known to those of skill in the art. In some embodiments, administrating of the cellular engineering target includes aerosol inhalation, injection, ingestion, transfusion, implantation or transplantation. In some embodiments, administrating of the cellular engineering target compositions includes transarterially, subcutaneously, intradermally, intratumorally, intranodally, intramedullary, intramuscularly, by intravenous (i.v.) injection, or intraperitoneally. In some embodiments, administrating of the cellular engineering target includes injection into a site of the subject, a local disease site in the subject, a lymph node, an organ, a tumor, and the like.

In some embodiments, administrating of the cellular engineering target is at a desired dosage, which includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the method includes administering the populations or sub-types of cells, such as CD8.sup.+ and CD4.sup.+ T cells, at or within a tolerated difference of a desired dose of total cells, such as a desired dose of T cells. In some embodiments, the desired dose is a desired number of cells or a desired number of cells per unit of body weight of the subject to whom the cells are administered, e.g., cells/kg. In some embodiments, the desired dose is at or above a minimum number of cells or minimum number of cells per unit of body weight. In some embodiments, among the total cells, the individual populations or sub-types are present at or near a desired output ratio (such as CD4.sup.+ to CD8.sup.+ ratio), e.g., within a certain tolerated difference or error of such a ratio.

In some embodiments, the method includes administrating of the cellular engineering target at or within a tolerated difference of a desired dose of one or more of the individual populations or sub-types of cells, such as a desired dose of CD4+ cells and/or a desired dose of CD8+ cells. In some embodiments, the desired dose is a desired number of cells of the sub-type or population, or a desired number of such cells per unit of body weight of the subject to whom the cells are administered, e.g., cells/kg. In some embodiments, the desired dose is at or above a minimum number of cells of the population or subtype, or minimum number of cells of the population or sub-type per unit of body weight. Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4.sup.+ to CD8.sup.+ cells, and/or is based on a desired fixed or minimum dose of CD4.sup.+ and/or CD8.sup.+ cells.

In some embodiments, the method includes administrating of the cellular engineering target to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650) million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 1.times.10.sup.5 cells/kg to about 1.times.10.sup.11 cells/kg, 10.sup.4, and at or about 10.sup.11 cells/kilograms (kg) body weight, such as between 10.sup.5 and 10.sup.6 cells/kg body weight, for example, at or about 1.times.10.sup.5 cells/kg, 1.5.times.10.sup.5 cells/kg, 2.times.10.sup.5 cells/kg, or 1.times.10.sup.6 cells/kg body weight. For example, in some embodiments, the cells are administered at, or within a certain range of error of, between at or about 10.sup.4 and at or about 10.sup.9 T cells/kilograms (kg) body weight, such as between 10.sup.5 and 10.sup.6 T cells/kg body weight, for example, at or about 1.times.10.sup.5 T cells/kg, 1.5.times.10.sup.5 T cells/kg, 2.times.10.sup.5 T cells/kg, or 1.times.10.sup.6 T cells/kg body weight. In other exemplary embodiments, a suitable dosage range of modified cells for use in a method of the present disclosure includes, without limitation, from about 1.times.10.sup.5 cells/kg to about 1.times.10.sup.6 cells/kg, from about 1.times.10.sup.6 cells/kg to about 1.times.10.sup.7 cells/kg, from about 1.times.10.sup.7 cells/kg about 1.times.10.sup.8 cells/kg, from about 1.times.10.sup.8 cells/kg about 1.times.10.sup.9 cells/kg, from about 1.times.10.sup.9 cells/kg about 1.times.10.sup.10 cells/kg, from about 1.times.10.sup.10 cells/kg about 1.times.10.sup.11 cells/kg. In an exemplary embodiment, a suitable dosage for use in a method of the present disclosure is about 1.times.10.sup.8 cells/kg. In an exemplary embodiment, a suitable dosage for use in a method of the present disclosure is about 1.times.10.sup.7 cells/kg. In other embodiments, a suitable dosage is from about 1.times.10.sup.7 total cells to about 5.times.10.sup.7 total cells. In some embodiments, a suitable dosage is from about 1.times.10.sup.8 total cells to about 5.times.10.sup.8 total cells. In some embodiments, a suitable dosage is from about 1.4.times.10.sup.7 total cells to about 1.1.times.10.sup.9 total cells. In an exemplary embodiment, a suitable dosage for use in a method of the present disclosure is about 7.times.10.sup.9 total cells. In an exemplary embodiment, a suitable dosage is from about 1.times.10.sup.7 total cells to about 3.times.10.sup.7 total cells.

In some embodiments, the dose of total cells and/or dose of individual sub-populations of cells is within a range of between at or about 1.times.10.sup.5 cells/m.sup.2 to about 1.times.10.sup.11 cells/m.sup.2. In an exemplary embodiment, the dose of total cells and/or dose of individual sub-populations of cells is within a range of between at or about 1.times.10.sup.7/m.sup.2 to at or about 3.times.10.sup.7/m.sup.2. In an exemplary embodiment, the dose of total cells and/or dose of individual sub-populations of cells is within a range of between at or about 1.times.10.sup.8/m.sup.2 to at or about 3.times.10.sup.8/m.sup.2. In some embodiments, the dose of total cells and/or dose of individual sub-populations of cells is the maximum tolerated dose by a given patient.

In some embodiments, the method includes administrating the cellular engineering target at or within a certain range of error of between at or about 10.sup.4 and at or about 10.sup.9 CD4.sup.+ and/or CD8.sup.+ cells/kilograms (kg) body weight, such as between 10.sup.5 and 10.sup.6 CD4.sup.+ and/or CD8.sup.+ cells/kg body weight, for example, at or about 1.times.10.sup.5 CD4.sup.+ and/or CD8.sup.+ cells/kg, 1.5.times.10.sup.5 CD4.sup.+ and/or CD8.sup.+ cells/kg, 2.times.10.sup.5 CD4.sup.+ and/or CD8.sup.+ cells/kg, or 1.times.10.sup.6 CD4.sup.+ and/or CD8.sup.+ cells/kg body weight. In some embodiments, the cells are administered at or within a certain range of error of, greater than, and/or at least about 1.times.10.sup.6, about 2.5.times.10.sup.6, about 5.times.10.sup.6, about 7.5.times.10.sup.6, or about 9.times.10.sup.6 CD4.sup.+ cells, and/or at least about 1.times.10.sup.6, about 2.5.times.10.sup.6, about 5.times.10.sup.6, about 7.5.times.10.sup.6, or about 9.times.10.sup.6 CD8+ cells, and/or at least about 1.times.10.sup.6, about 2.5.times.10.sup.6, about 5.times.10.sup.6, about 7.5.times.10.sup.6, or about 9.times.10.sup.6 T cells. In some embodiments, the cells are administered at or within a certain range of error of between about 10.sup.8 and 10.sup.12 or between about 10.sup.10 and 10.sup.11 T cells, between about 10.sup.8 and 10.sup.12 or between about 10.sup.10 and 10.sup.11 CD4.sup.+ cells, and/or between about 10.sup.8 and 10.sup.12 or between about 10.sup.10 and 10.sup.11 CD8.sup.+ cells.

In some embodiments, the method includes administrating the cellular engineering target with a toleration range of a desired output ratio of multiple cell populations or sub-types, such as CD4+ and CD8+ cells or sub-types. In some embodiments, the desired ratio is a specific ratio or can be a range of ratios, for example, in some embodiments, the desired ratio (e.g., ratio of CD4.sup.+ to CD8.sup.+ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1. 1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some embodiments, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, the method includes administrating the cellular engineering target a dose of modified cells in a single dose or multiple doses. In some embodiments, administrating the cellular engineering includes multiple doses, e.g., once a week or every 7 day's, once every 2 weeks or every 14 days, once every 3 weeks or every 21 days, once every 4 weeks or every 28 days. In an exemplary embodiment, administrating the cellular engineering includes a single dose of modified cells, such as by rapid intravenous infusion.

In some embodiments, for the prevention or treatment of disease, the appropriate dosage depends on the type of disease, the type of cells or recombinant receptors, the severity and course of the disease, whether administrating the cells for preventive or therapeutic purposes, previous therapy, the subject's clinical history and response to the cellular engineering target, and the discretion of the attending physician. In some embodiments, the method includes administrating the compositions and cells once or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

In some embodiments, the method includes determining the biological activity of the cellular engineering target, e.g., by any of a number of known methods. In some embodiments, one or more parameters utilized in such a determination include specific binding of an engineered or natural T cell or other immune cell to antigen, in vivo, e.g., by imaging, or ex vivo, e.g., by ELISA or flow cytometry. In certain embodiments, the method includes determining the ability of the engineered cells to destroy target cells using any suitable method known in the art, such as cytotoxicity assays. In certain embodiments, the method includes determining the biological activity of the cells by assaying expression and/or secretion of one or more cytokines, such as CD 107a, IFNy, IL-2, and TNF. In some embodiments, the method includes determining the biological activity by assessing clinical outcome, such as reduction in tumor burden or load.

In some embodiments, the method includes providing a specific dosage regimen that includes a lymphodepletion step prior to the administration of the modified T cells. In an exemplary embodiment, the lymphodepletion step includes administrating cyclophosphamide and/or fludarabine.

In some embodiments, the administrating of lymphodepletion includes administrating cyclophosphamide at a dose of between about 200 mg/m.sup.2/day and about 2000 mg/m.sup.2/day (e.g., 200 mg/m.sup.2/day, 300 mg/m.sup.2/day, or 500 mg/m.sup.2/day). In an exemplary embodiment, the dose of cyclophosphamide is about 300 mg/m.sup.2/day. In some embodiments, the lymphodepletion step includes administration of fludarabine at a dose of between about 20 mg/m.sup.2/day and about 900 mg/m.sup.2/day (e.g., 20 mg/m.sup.2/day. 25 mg/m.sup.2/day, 30 mg/m.sup.2/day, or 60 mg/m.sup.2/day). In an exemplary embodiment, the dose of fludarabine is about 30 mg/m.sup.2/day.

In some embodiment,, the administrating of lymphodepletion includes administrating cyclophosphamide at a dose of between about 200 mg/m.sup.2/day and about 2000 mg/m.sup.2/day (e.g., 200 mg/m.sup.2/day, 300 mg/m.sup.2/day, or 500 mg/m.sup.2/day), and fludarabine at a dose of between about 20 mg/m.sup.2/day and about 900 mg/m.sup.2/day (e.g., 20 mg/m.sup.2/day, 25 mg/m.sup.2/day, 30 mg/m.sup.2/day, or 60 mg/m.sup.2/day). In an exemplary embodiment, the administrating of lymphodepletion includes administrating cyclophosphamide at a dose of about 300 mg/m.sup.2/day, and fludarabine at a dose of about 30 mg/m.sup.2/day.

In an exemplary embodiment, a subject has a diagnosis for castrate-resistant prostate cancer, the method includes administrating lymphodepleting chemotherapy prior to administrating of the modified T cellular engineering target. In an exemplary embodiment, for a subject having castrate-resistant prostate cancer, the subject receives lymphodepleting chemotherapy including at or about 500 mg/m.sup.2 to at or about 1 g/m.sup.2 of cyclophosphamide by intravenous infusion. In an exemplary embodiment, for a subject having castrate-resistant prostate cancer, the subject receives lymphodepleting chemotherapy including at or about 500 mg/m.sup.2 to at or about 1 g/m.sup.2 of cyclophosphamide by intravenous infusion about 3 days (.+−.1 day) prior to administration of the modified T cells. In an exemplary embodiment, for a subject having castrate-resistant prostate cancer, the subject receives lymphodepleting chemotherapy including at or about 500 mg/m.sup.2 to at or about 1 g/m.sup.2 of cyclophosphamide by intravenous infusion up to 4 days prior to administration of the modified T cells. In an exemplary embodiment, for a subject having castrate-resistant prostate cancer, the subject receives lymphodepleting chemotherapy including at or about 500 mg/m.sup.2 to at or about 1 g/m.sup.2 of cyclophosphamide by intravenous infusion 4 days prior to administration of the modified T cells. In an exemplary embodiment, for a subject having castrate-resistant prostate cancer, the subject receives lymphodepleting chemotherapy including at or about 500 mg/m.sup.2 to at or about 1 g/m.sup.2 of cyclophosphamide by intravenous infusion 3 days prior to administration of the modified T cells. In an exemplary embodiment, for a subject having castrate-resistant prostate cancer, the subject receives lymphodepleting chemotherapy including at or about 500 mg/m.sup.2 to at or about 1 g/m.sup.2 of cyclophosphamide by intravenous infusion 2 days prior to administration of the modified T cells.

In an exemplary embodiment, the method includes, a subject having castrate-resistant prostate cancer, administrating lymphodepleting chemotherapy including 300 mg/m.sup.2 of cyclophosphamide by intravenous infusion 3 days prior to administrating the modified T cells. In an exemplary embodiment, for a subject having castrate-resistant prostate cancer, the method includes administrating lymphodepleting chemotherapy including 300 mg/m.sup.2 of cyclophosphamide by intravenous infusion for 3 days prior to administrating the modified T cells.

In an exemplary embodiment, for a subject having castrate-resistant prostate cancer, the method includes administrating lymphodepleting chemotherapy including fludarabine at a dose of between about 20 mg/m.sup.2/day and about 900 mg/m.sup.2/day (e.g., 20 mg/m.sup.2/day, 25 mg/m.sup.2/day, 30 mg/m.sup.2/day, or 60 mg/m.sup.2/day). In an exemplary embodiment, for a subject having castrate-resistant prostate cancer, the method includes administrating lymphodepleting chemotherapy including fludarabine at a dose of 30 mg/m.sup.2 for 3 days.

In an exemplary embodiment, for a subject having castrate-resistant prostate cancer, the method includes administrating lymphodepleting chemotherapy including cyclophosphamide at a dose of between about 200 mg/m.sup.2/day and about 2000 mg/m.sup.2/day (e.g., 200 mg/m.sup.2/day, 300 mg/m.sup.2/day, or 500 mg/m.sup.2/day), and fludarabine at a dose of between about 20 mg/m.sup.2/day and about 900 mg/m.sup.2/day (e.g., 20 mg/m.sup.2/day, 25 mg/m.sup.2/day, 30 mg/m.sup.2/day, or 60 mg/m.sup.2/day). In an exemplary embodiment, for a subject having castrate-resistant prostate cancer, the method includes administrating lymphodepleting chemotherapy including cyclophosphamide at a dose of about 300 mg/m.sup.2/day, and fludarabine at a dose of 30 mg/m.sup.2 for 3 days.

It is known in the art that one of the adverse effects following infusion of CAR T cells is the onset of immune activation, known as cytokine release syndrome (CRS). CRS is immune activation resulting in elevated inflammatory cytokines. Clinical and laboratory measures range from mild CRS (constitutional symptoms and/or grade-2 organ toxicity) to severe CRS (sCRS; grade.gtoreq.3 organ toxicity, aggressive clinical intervention, and/or potentially life threatening). Clinical features include high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been shown following CAR T-cell infusion. The presence of CRS generally correlates with expansion and progressive immune activation of adoptively transferred cells. It has been demonstrated that the degree of CRS severity is dictated by disease burden at the time of infusion as patients with high tumor burden experience a more sCRS.

Accordingly, the present disclosure provides for, following the diagnosis of CRS, appropriate CRS management strategies that mitigate one or more physiological symptoms of uncontrolled inflammation without dampening the antitumor efficacy of the cellular engineering target (e.g., CAR T cells). CRS management strategies are known in the art. For example, in some embodiments, the method includes administrating systemic corticosteroids to rapidly reverse symptoms of sCRS (e.g., grade 3 CRS) without compromising initial antitumor response.

In some embodiments, the method includes administrating an anti-IL-6R antibody. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administrating of tocilizumab has demonstrated near-immediate reversal of CRS.

In some embodiments, the method includes selecting and treating a subject having failed at least one prior course of standard of cancer therapy. For example, a suitable subject may have had a confirmed diagnosis of relapsed prostate cancer. In some embodiments, the method includes selecting and treating a subject having had at least one prior course of standard of cancer therapy. For example, a suitable subject may have had prior therapy with at least one standard 17.alpha. lyase inhibitor or second-generation anti-androgen therapy for the treatment of metastatic castrate resistant prostate cancer.

In an exemplary embodiment, a suitable subject is a subject having metastatic castrate resistant prostate cancer. In an exemplary embodiment, a suitable subject is a subject having metastatic castrate resistant prostate cancer having .gtoreq.10% tumor cells expressing PSMA as determined by immunohistochemistry analysis on fresh tissue.

In some embodiments, a suitable subject is a subject that has radiographic evidence of osseous metastatic disease and/or quantifiable, non-osseous metastatic disease (nodal or visceral).

In some embodiments, a suitable subject includes an ECOG performance status of 0-1.

In some embodiments, a suitable subject exhibits adequate organ function, as defined by: serum creatinine.ltoreq.1.5 mg/dl or creatinine clearance.gtoreq.60 cc/min; and/or serum total bilirubin<1.5.times.ULN; serum ALT/AST<2.times.ULN.

In some embodiments, a suitable subject exhibits adequate hematologic reserve as defined by: Hgb>10 g/dl; PLT>100 k/ul; and/or ANC>1.5 k/ul.

In some embodiments, a suitable subject is not transfusion dependent.

In some embodiments, a suitable subject is a subject that has evidence of progressive castrate resistant prostate adenocarcinoma, as defined by: castrate levels of testosterone(<50 ng/ml) with or without the use of androgen deprivation therapy; and/or evidence of one of the following measures of progressive disease: soft tissue progression by RECIST 1.1 criteria, osseous disease progression with 2 or more new lesions on bone scan (as per PCWG2 criteria), increase in serum PSA of at least 25% and an absolute increase of 2 ng/ml or more from nadir (as per PCWG2 criteria).

In some embodiments, a suitable subject has had previous treatment with at least one second-generation androgen signaling inhibitor. In some embodiments, a suitable subject has had previous treatment with abiraterone. In some embodiments, a suitable subject has had previous treatment with enzalutamide.

In some embodiments, a suitable subject includes .gtoreq.10% tumor cells expressing PSMA by immunohistochemistry (IHC) on a metastatic tissue biopsy.

In some embodiments, a suitable subject includes radiographic evidence for metastatic disease (osseous or nodal/visceral).

In some embodiments, a suitable subject includes .ltoreq.4 lines of therapy for metastatic CRPC.

Additional details and information regarding the manufacture of cellular engineering targets can be found at U.S. Pat. No. 10,780,120, entitled “Prostate-specific membrane antigen cars and methods of use thereof,” filed Mar. 5, 2019; U.S. Pat. No. 10,839,945, entitled “Cell processing method,” filed Jul. 6, 2015; U.S. Pat. No. 10,428,351, entitled “Methods for transduction and cell processing,” filed Nov. 4, 2015; U.S. Pat. No. 10,877,055, entitled “Parallel cell processing method and facility,” filed Jan. 11, 2019, each of which is hereby incorporated by reference in its entirety for all purposes.

Referring to FIG. 2, the computer system 100 is configured to store an instrument library 106 describing a plurality of instruments 52 of a respective modular biological foundry system, 200.

In some embodiments, the server 200 receives the data elements wirelessly through radio-frequency (RF) signals. In some embodiments, such signals are in accordance with an 802.11 (Wi-Fi), Bluetooth, or ZigBee standard.

In some embodiments, the computer system 100 is not proximate to the biological foundry 200 and/or does not have wireless capabilities or such wireless capabilities are not used for the purpose of providing instructions to the instruments 300 of the biological foundry 200. In such embodiments, a communication network 106 is utilized to communicate an update for executing a respective instance of a corresponding compiled workflow from the service to the biological foundry. In some embodiments, the communication network 106 is utilized to communicate a result of a manufacture of a respective cellular engineering target produced at the biological foundry 200 to the computer system 100.

Examples of communication networks 106 include, but are not limited to, the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of the present disclosure.

Of course, other topologies of the system 10 other than the one depicted in FIG. 1 are possible. For instance, in some embodiments, rather than relying on a communications network 106, the computer system 100 wirelessly transmits information directly to the biological foundry 200. Further, in some embodiments, the computer system 100 constitutes a portable electronic device, a server computer, or in fact constitute several computers that are linked together in a network or are instantiated as one or virtual machines and/or containers in a cloud-computing context. As such, the exemplary topology shown in FIG. 1 merely serves to describe the features of an embodiment of the present disclosure in a manner that will be readily understood to one of skill in the art.

Additional details and information regarding workflows at a biological foundry system can be found at International Patent Application no.: PCT/US2021/018927, entitled “Systems and Methods for Facilitating Modular and Parallelized Manufacturing at a Biological Foundry,” filed Feb. 19, 2021, which is hereby incorporated by reference in its entirety for all purposes.

Accordingly, one aspect of the present disclosure is directed to providing systems, methods, and apparatuses that facilitates providing a modular biological foundry system. The modular biological foundry system includes a controller and a communications interface that is in electrical communication with the controller. Moreover, the modular biological foundry system includes a plurality of peripheral devices, which in turn includes an articulated handling robot and a power supply. The articulated handling robot is configured to move a cell therapy cartridge between at least a first biological foundry instrument and a second biological foundry instrument in a plurality of biological foundry instruments configured to produce a portion of the one or more cellular engineering targets. The modular system includes a frame surrounding the articulated handling robot. The frame includes at least two modules in a plurality of modules. A first module in the at least two modules is configured to accommodate a respective biological foundry instrument in the plurality of biological foundry instructions. Moreover, each module in the plurality of modules includes a plurality of elongated members. Each module in the plurality of modules further includes a first plurality of coupling mechanisms for coupling at least two elongated members in the plurality of elongated members. Additionally, each module in the plurality of modules includes a second plurality of coupling mechanisms for removably coupling a respective elongated member in the plurality of elongated members with the frame. Furthermore, each module in the plurality of modules includes a plurality of walls engaged with and supported by the at least two elongated members in the plurality of elongated members. Accordingly, the plurality of walls forms an internal volume of a respective module sealed from an environment, such that a modular biological foundry system is provided.

In some embodiments, the modular biological foundry system further includes a transport path coupled to the articulated handling robot and in electrical communication with the communications interface. The transport path extends from a first end portion of a first wall in the plurality of walls to a second end portion of a second wall in the plurality of walls.

With the foregoing in mind, consider that a manufacturing process for a cellular engineering target is divided into a plurality of steps. In each step, a different manufacturing task or sub-process is carried out. For instance, in some embodiments, the different manufacturing task includes incubation of cell culture, isolation of one or more types of cells, viral transduction, freezing, thawing, etc. Accordingly, the present disclosure provides a modular clean room biological foundry system that utilizes a separate module to conduct each step of the manufacturing process.

Each module 208 includes an instrument (e.g., instrument 300 of FIG. 1) that performs a particular step (or set of steps) in a cellular engineering target manufacturing process. Accordingly, in some embodiments, each module includes both the instrument that carries out the task, and/or one or more support instruments for that task. Support instruments includes air filtering systems, electronic systems, processors (e.g., CPU 202 of FIG. 2), cloud connectivity devices (e.g., communications network 106 of FIG. 1), and one or more reservoirs of raw material or waste material of the manufacturing process. A module has a well-defined shape formed through elongated members, which allows for the module to change positions within the modular biological foundry without requiring modification to the frame of the modular biological foundry system. Accordingly, a plurality of modules, together, realize the manufacturing process for producing a plurality of cellular engineering targets.

Additional details and information regarding workflows at a biological foundry system can be found at International Patent Application no.: PCT/US21/28031, entitled “Modular Robotic System and Modular Closed-System Architecture for the Parallel, Automatic Manufacturing Of Cell Therapies,” filed April 19, 2021, which is hereby incorporated by reference in its entirety for all purposes.

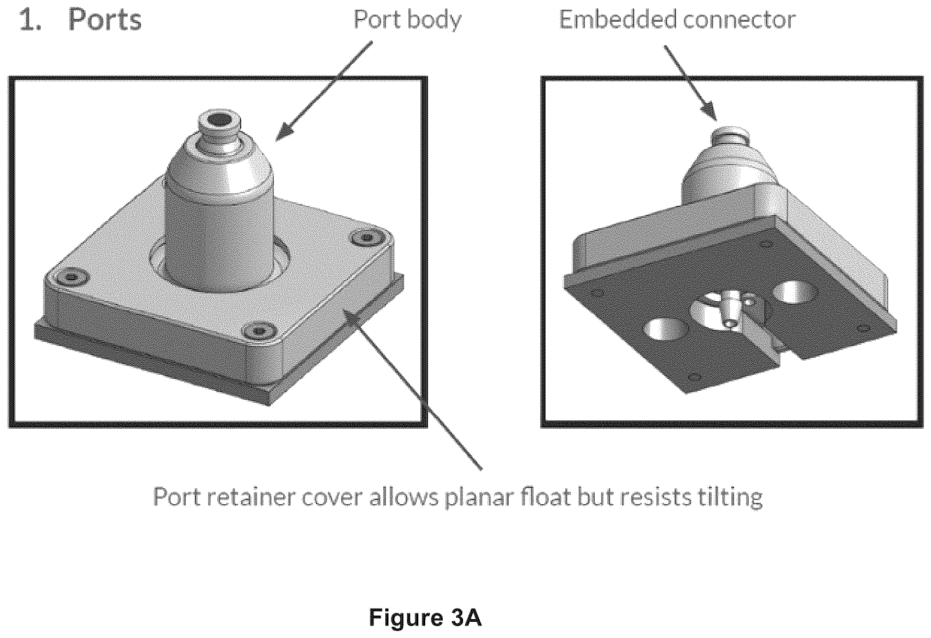

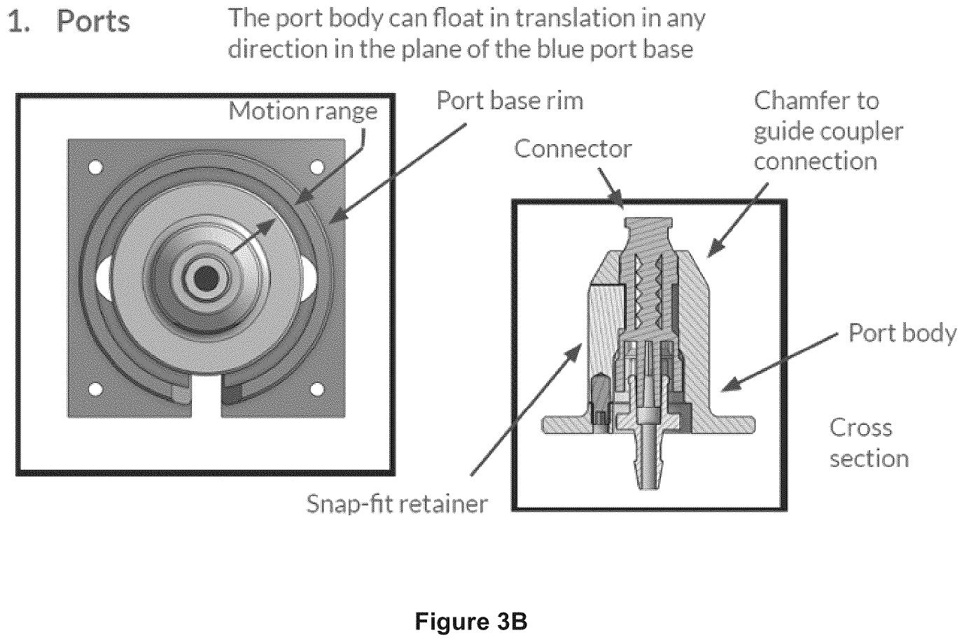

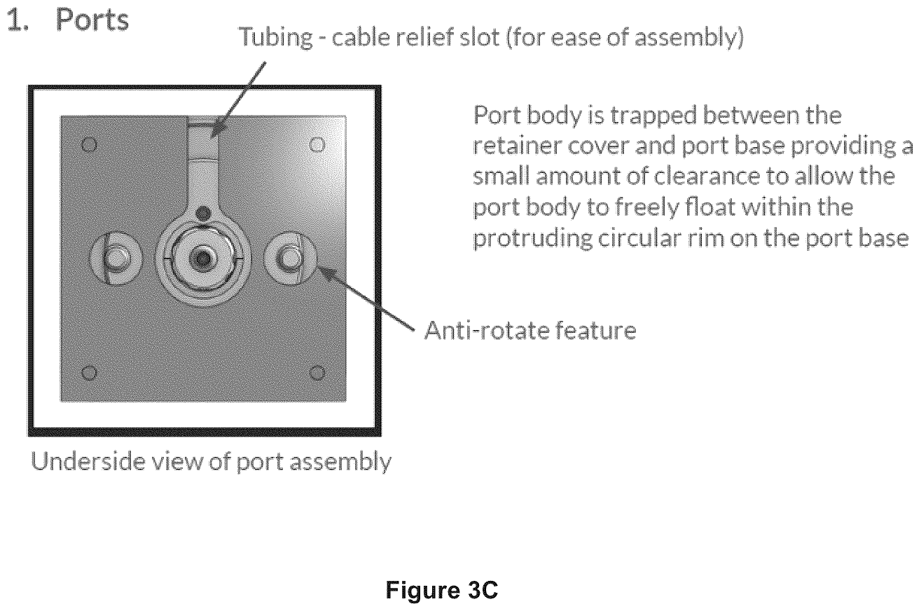

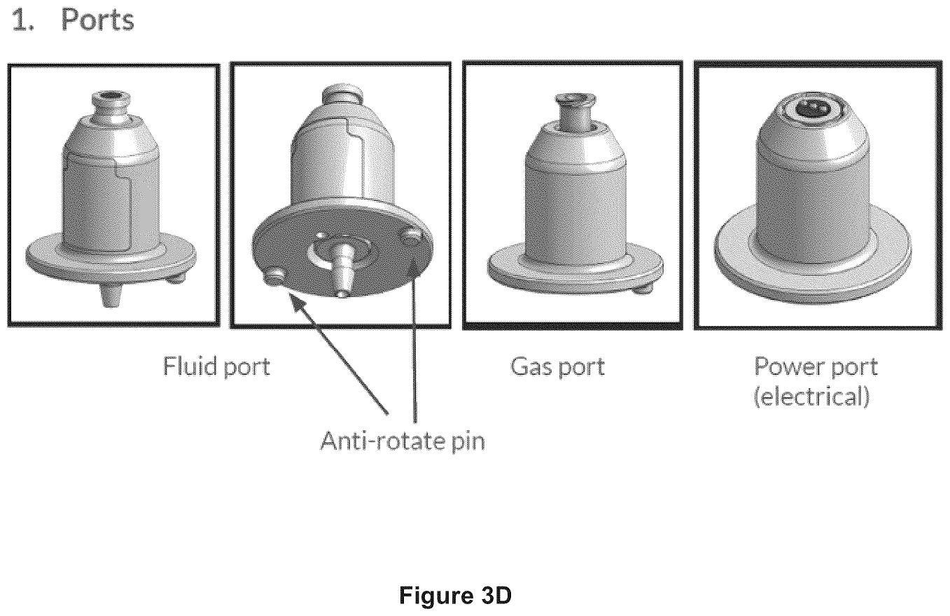

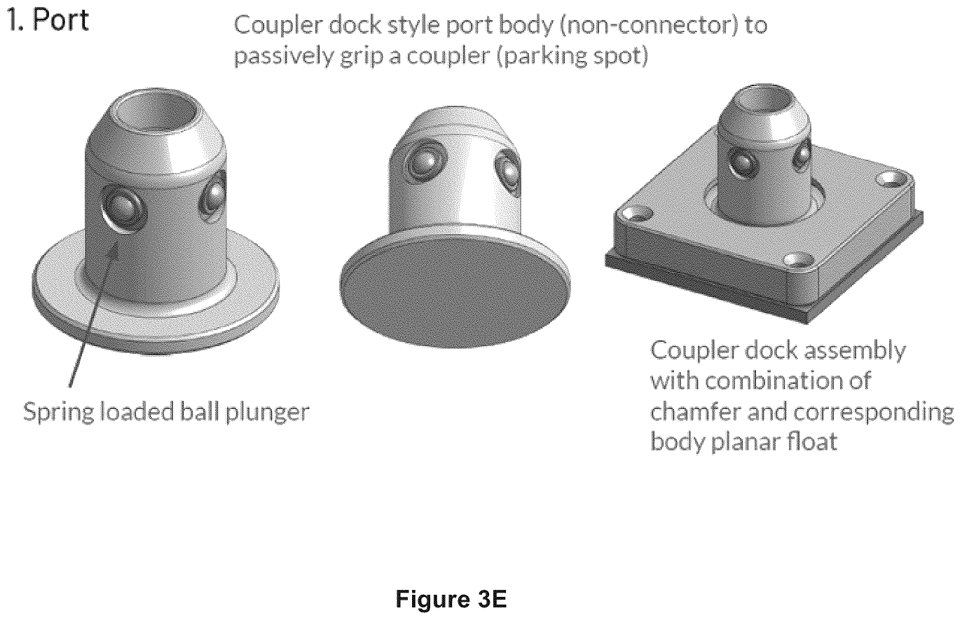

FIGS. 3A-3E illustrate an exemplary port in accordance with some exemplary embodiments of the present disclosure. In various embodiments, the port includes a floating non-rotating design that is compatible with a variety of fluid and non-fluid connectors and incorporates an external lead-in feature for the mating coupler. One of the key innovations is the combination of the port body shape which has a large chamfered tip to engage with a mating coupler and corresponding size multi-directional planar float of the port body that accommodates axial misalignment but resists torsional loads typically associated with a threaded connector. The port body is designed to house fluid, gas, and electrical connectors effectively converting an array of industry standard connectors into robotic compatible connectors due to the external mating feature and position tolerance compensation via port body float.

In some embodiments, the port is configured for accommodating axial misalignment during mating with a part. The port includes a retainer and a port body. The retainer includes: a first retaining member having a first surface; a second retaining member having a second surface spaced apart from the first surface of the first retaining member in a first direction, wherein the first retaining member and the second retaining member are connected to each other; a first circular through-hole formed on the first retaining member; and a circular rim formed on the first surface of the first retaining member or the second surface of the second retaining member, and concentric with the first circular through-hole. The port body includes: a circular base disposed between the first surface of the first retaining member and the second surface of the second retaining member; a circular stem extending from the circular base in the first direction and passing through the first circular through-hole formed on the first retaining member; and a chamfered tip formed at a free end portion of the circular stem and configured for guiding the port body to mate with the part. In some such embodiments, (i) an outer diameter of the circular base is larger than a diameter of the first circular through-hole but smaller than an inner diameter of the circular rim and (ii) an outer diameter of the circular stem is smaller than the diameter of the first circular through-hole, such that the port body is movable relative to the retainer in one or more directions substantially perpendicular to the first direction and within a range bounded by the circular rim, the first circular through-hole, or both of the circular rim and the first circular through-hole.

In some embodiments, the part is a coupler.

In some embodiments, the second retaining member is an individual piece.

In some embodiments, the second retaining member is a wall of a device.

In some embodiments, the circular rim is formed on the second surface of the second retaining member.

In some embodiments, the circular rim does not form a closed circle.

In an exemplary embodiment, the circular rim includes one or more rim segments.

In some embodiments, the circular base is substantially planar.

In an exemplary embodiment, a thickness of the circular base equals substantially a distance between the first surface of the first retaining member and the second surface of the second retaining member.

In some embodiments, the retainer further includes one or more first anti-rotation members. In some embodiments, the port body further includes one or more second anti-rotation members coupled to the one or more first anti-rotation members to restrict the port body from rotating relative to the retainer around an axis of the port body.

In some embodiments, each of the one or more first anti-rotation members is a hole formed on the second retaining member. In some embodiments, each of the one or more second anti-rotation members is a pin formed on the circular base and inserted into a corresponding hole formed on the first retaining member. In some embodiments, a diameter of the pin is smaller than a diameter of the corresponding hole formed on the second retaining member.

In an exemplary embodiment, each of the one or more first anti-rotation members is formed adjacent to an inner edge of the circular rim. In some embodiments, each of the one or more second anti-rotation members is formed adjacent to an outer edge of the circular base.

In some embodiments, the port body is hollow for housing a connector.

In some embodiments, the connector includes a fluid connector, a gas connector, an electrical connector, or any combination thereof.

In some embodiments, one or more first engaging members are disposed on the circular stem of the port body and further configured to engage with the connector.

In an exemplary embodiment, an engaging member in the one or more first engaging members is a snap-fit.

In some embodiments, a second circular through-hole is formed on the second retaining member and concentric with the first circular through-hole formed on the first retaining member.

In some embodiments, one or more slots are formed at the first or second retaining member for each of assembly.

In some embodiments, one or more second engaging members are disposed on the circular stem of the port body and further configured for engaging with the part.

In an exemplary embodiment, an engaging member in the one or more second engaging members is a spring loaded ball plunger.

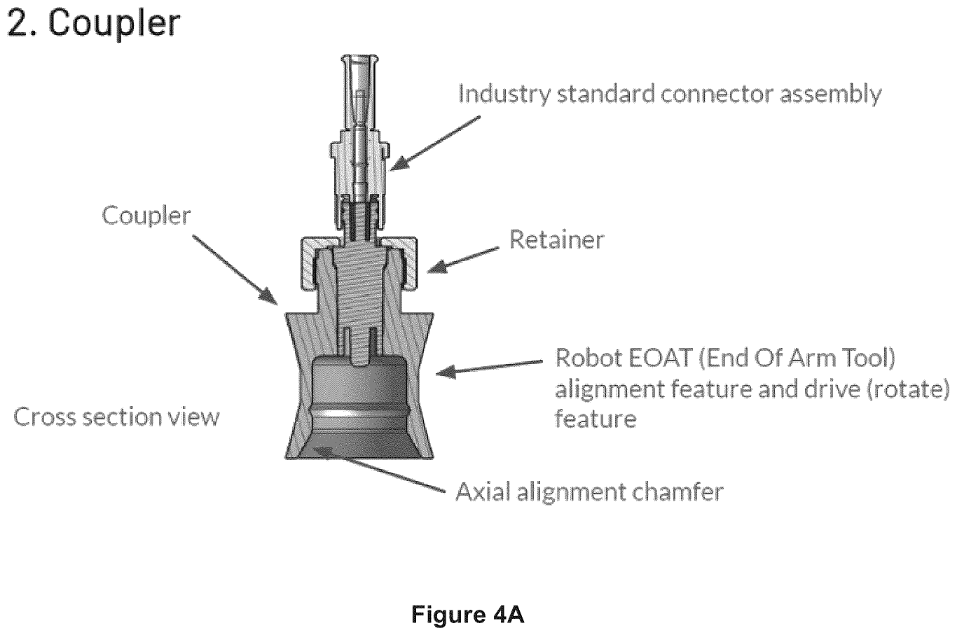

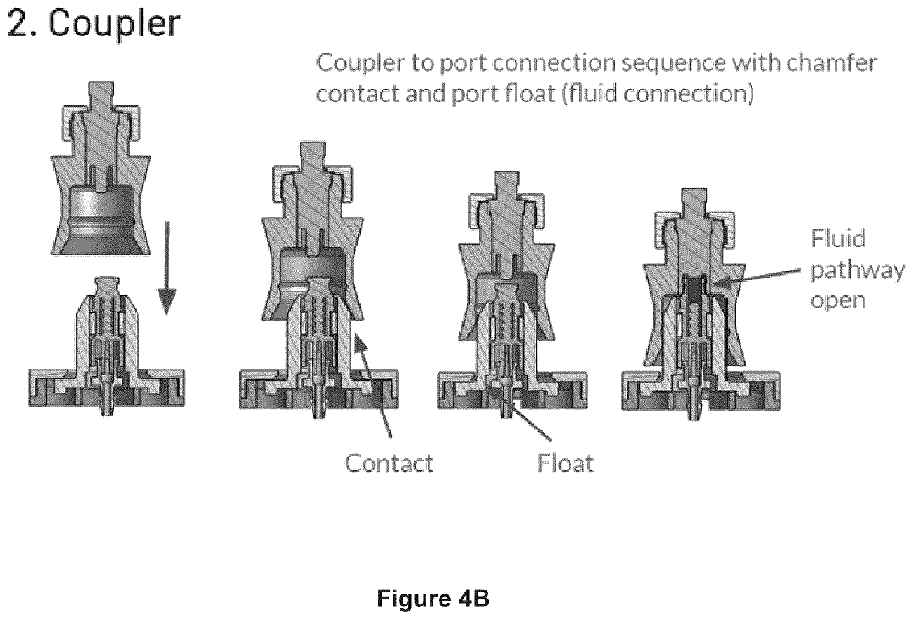

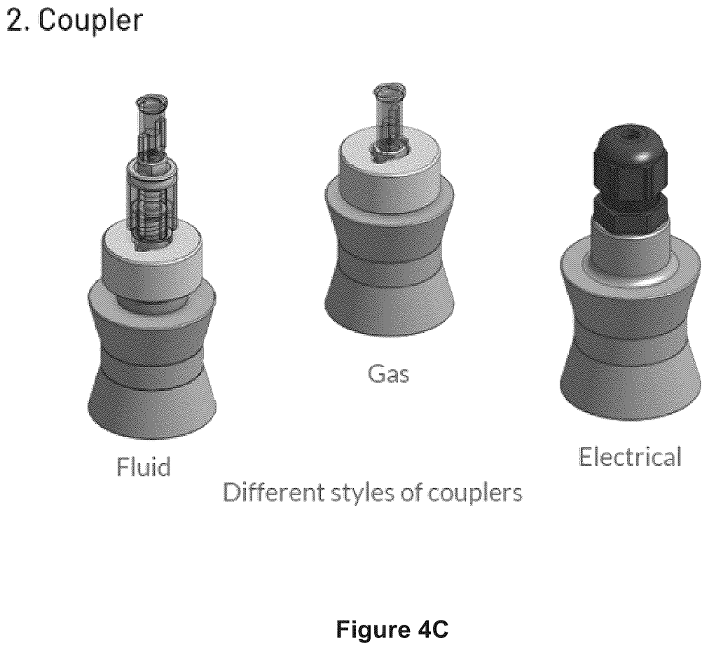

FIGS. 4A-4C illustrate an exemplary coupler in accordance with some exemplary embodiments of the present disclosure. In various embodiments, the coupler includes a dual function design with external drive and alignment features and internal features for compatibility with a variety of fluid and non-fluid connectors. In some embodiments, one of the key innovations is the dual function aspect of the design that can convert a variety of industry standard connectors into robot-grippable-robot-drivable (twist) connectors with specific features to accommodate axial misalignment during mating with the corresponding port.

In some embodiments, the coupler is configured for converting a first connector to a robot-operable connector. In some embodiments, the coupler includes a rotational axis; a coupling section for connecting the first connector to the coupler. In some embodiments, the couple includes a robot-operable section including a revolving exterior surface around the rotational axis of the coupler. In some such embodiments, (i) the revolving exterior surface includes a first segment proximal to the coupling section and a second segment distal to the coupling section, (ii) each of the first and second segments of the revolving exterior surface has a wide side and a narrow side, and (iii) the narrow sides of the first and second segments of the revolving exterior surface face each other, thereby providing an interface for a robotic end of arm tool (EOAT) to grip, hold and/or rotate the coupler.

In some embodiments, the first connector is an industrial standard connector.

In some embodiments, the first connector includes a fluid connector, a gas connector, an electrical connector, or any combination thereof.

In some embodiments, the coupling section is connected to the first connector by a retainer.

In an exemplary embodiment, the retainer is a component of the first connector.

In some embodiments, the first and second segments of the revolving exterior surface are substantially the same in size and shape.

In some embodiments, one of the first and second segments of the revolving exterior surface is a conical surface. In some embodiments, the other of the first and second segments of the revolving exterior surface is an inverted conical surface.

In some embodiments, the revolving exterior surface further includes a third segment between the first and second segments and connecting the narrow side of the first segment with the narrow side of the second segment.

In an exemplary embodiment, the third segment of revolving exterior surface is a cylindrical surface.

In some embodiments, the coupler is hollow for housing at least a portion of the first connector.

In some embodiments, the robot-operable section includes an inner chamfer formed at a free end portion of the robot-operable section to guide connection of the coupler with a port and/or accommodate axial misalignment during connection of the coupler with the port.

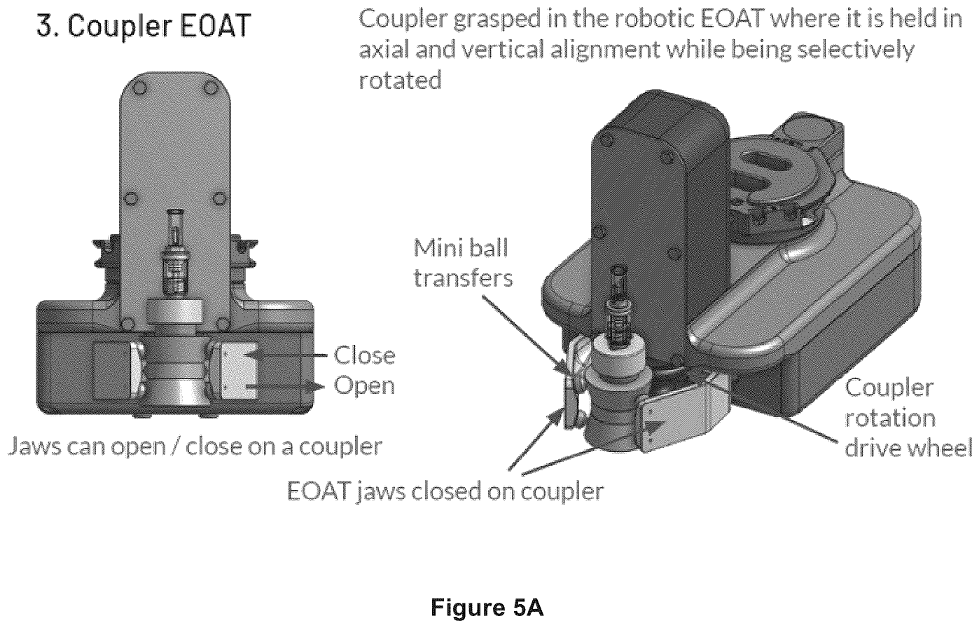

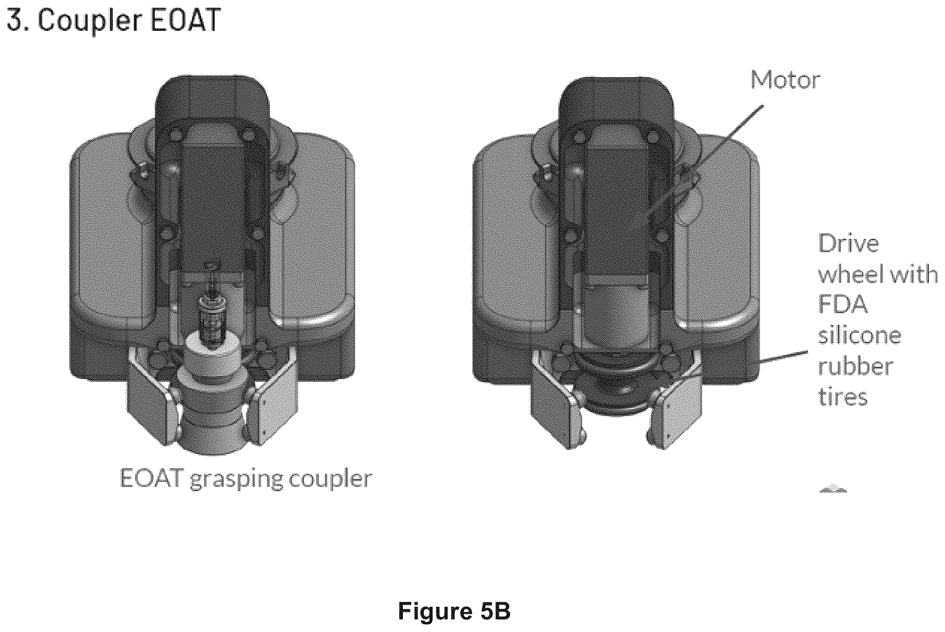

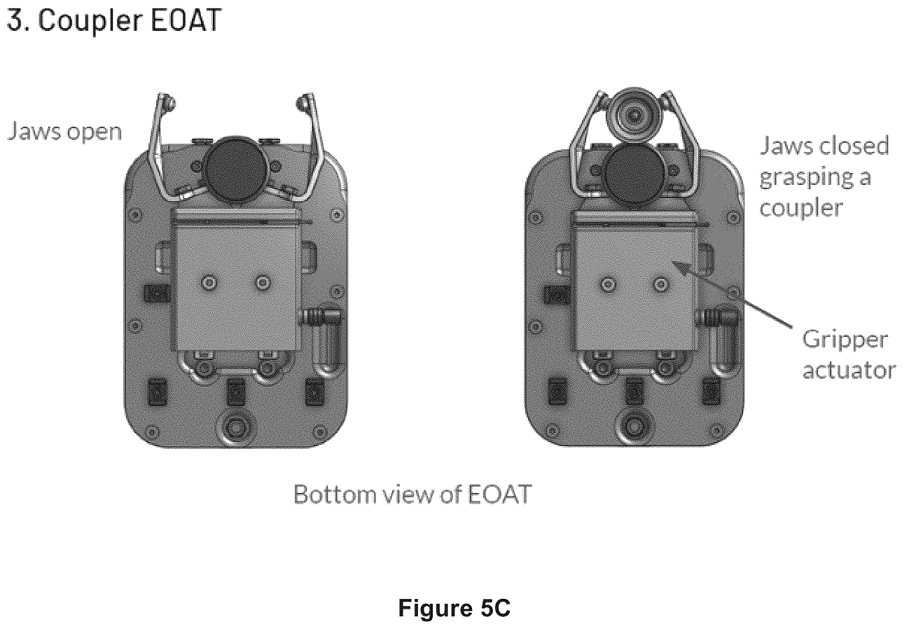

FIGS. 5A-5C illustrate a first exemplary robotic end of arm tool (EOAT) in accordance with some exemplary embodiments of the present disclosure. This robotic EOAT includes a novel robotic coupler grasp and rotate mechanism. In some embodiments, the combination of axial grasping with coupler position control along the rotation axis, and the simultaneous ability to rotate the grasped coupler via a friction drive wheel against the exterior of the coupler (hold and rotate the part while the EOAT body remains stationary).

In some embodiments, this robotic EOAT is configured for operating a part that includes a revolving exterior surface around a rotational axis of the part. In some embodiments, the revolving exterior surface includes a first non-cylindrical segment and a second non-cylindrical segment. In some embodiments, the robotic EOAT includes a support, a first jaw, a second jaw; and a wheel. The first jaw and the second jaw are connected to the support and operable between an open position and a closed position for gripping and releasing the part. In some embodiments, each of the first and second jaws includes a first contact bearing and a second contact bearing. The wheel is connected to the support and operable to rotate around a rotational axis of the wheel, wherein the wheel includes a first rim and a second rim. When the first and second jaws are in the closed position with the part in between: the first contact bearing of the first jaw, the first contact bearing of the second jaw and the first rim of the wheel are leveled substantially with each other and abut the first non-cylindrical segment of the revolving exterior surface of the part, and the second contact bearing of the first jaw, the second contact bearing of the second jaw and the second rim of the wheel are leveled substantially with each other and abut the second non-cylindrical segment of the revolving exterior surface of the part, thereby (i) restricting the part from moving axially, (ii) restricting the part from moving translationally in a plane substantially perpendicular to the rotational axis of the part, and (iii) allowing the part to rotate around the rotational axis of the part by the wheel.

In some embodiments, the part is the coupler disclosed herein (e.g., the coupler disclosed herein with respect to FIGS. 4A-4C).

In some embodiments, the first and second jaws are substantially symmetrical to each other with respect to the wheel.

In some embodiments, the first jaw; the second jaw and the wheel are disposed at a side of the support.

In some embodiments, the first and second contact bearings of the first or second jaw are aligned in a direction substantially parallel to the rotational axis of the wheel.

In some embodiments, a bearing in the first and second contact bearings of the first and second jaws is a ball of a ball transfer unit.

In some embodiments, each of the first and second jaws includes a jaw surface, and the first and second contact bearings are disposed at the jaw surface.

In an exemplary embodiment, the jaw surface is profiled in accordance with the revolving exterior surface of the part.

In some embodiments, each of the first and second rims of the wheel includes a silicone rubber tire.

In some embodiments, the robotic EOAT further includes an actuator to open and close the first and second jaws, a motor to drive the wheel, or both of the actuator and the motor.

In an exemplary embodiment, the first and second jaws are connected to the support through the actuator, and the wheel is connected to the support through the motor.

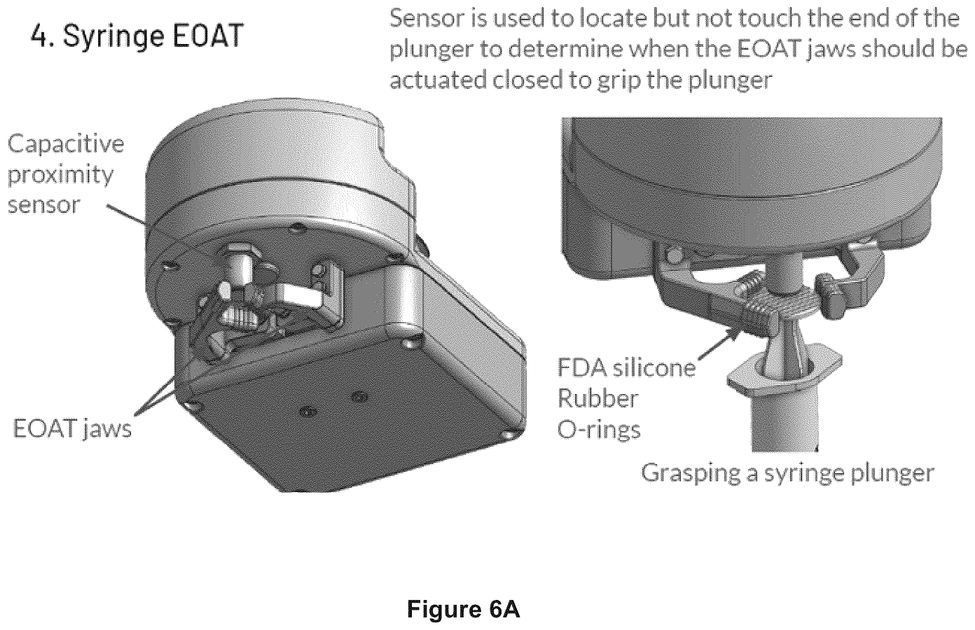

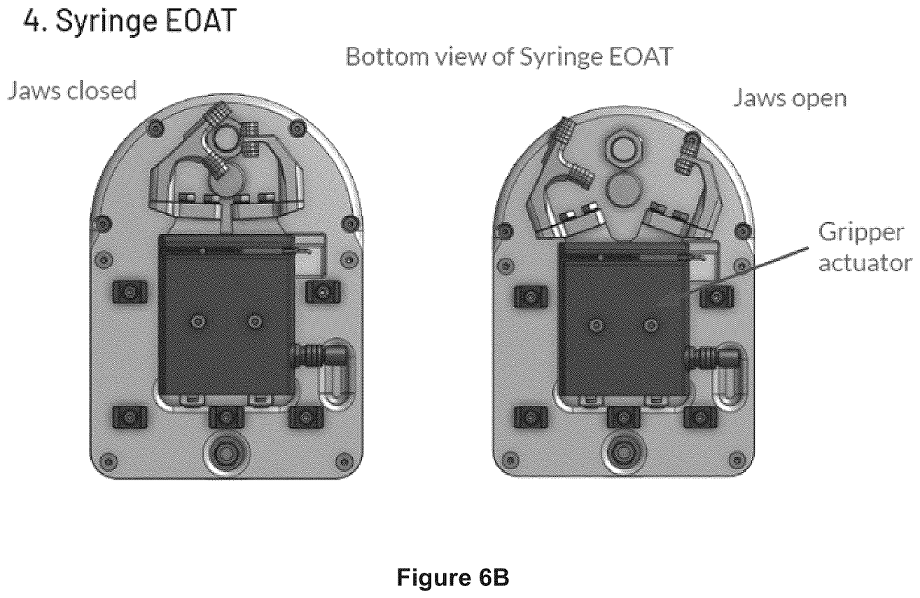

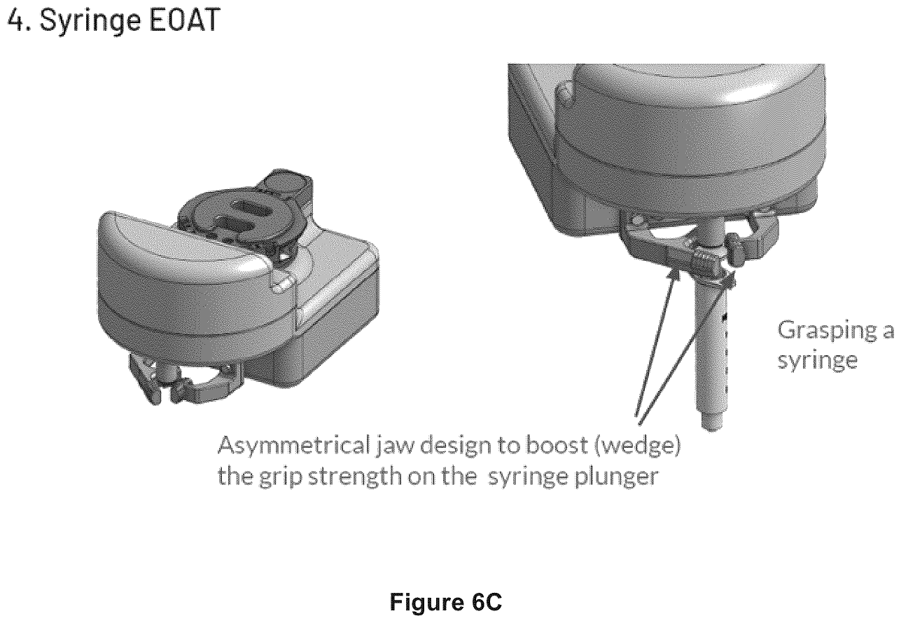

FIGS. 6A-6B illustrate a second exemplary robotic EOAT in accordance with some exemplary embodiments of the present disclosure. In various embodiments, this robotic EOAT includes a novel syringe plunger grasp mechanism that accommodates misalignment and prevents unintentional movement of the syringe piston during gripping. In some embodiments, the combination of a sensor to detect the position of a syringe plunger and the jaw profiles with FDA silicone rubber friction bands enable the syringe EOAT to grip the end of the plunger with uncertain position information and still not move the plunger position relative to the syringe body.

In some embodiments, the robotic EOAT is configured for operating a part. The robotic EOAT includes a support, a first jaw, a second jaw, and a sensor. In some embodiments, the first jaw and the second jaw are connected to the support and operable between an open position and a closed position for gripping and releasing a part. In some embodiments, the sensor is disposed at the support and configured to detect a proximity of the part and provide a signal to actuate the first and second jaws.

In some embodiments, the part is a plunger of a syringe.

In some embodiments, each of the first and second jaws includes a gripping segment and a plurality of silicone rubber O-rings disposed on the gripping segment.

In an exemplary embodiment, the gripping segment of the first jaw is substantially straight and the gripping segment of the second jaw is curved.

In some embodiments, the first jaw is different than the second jaw.

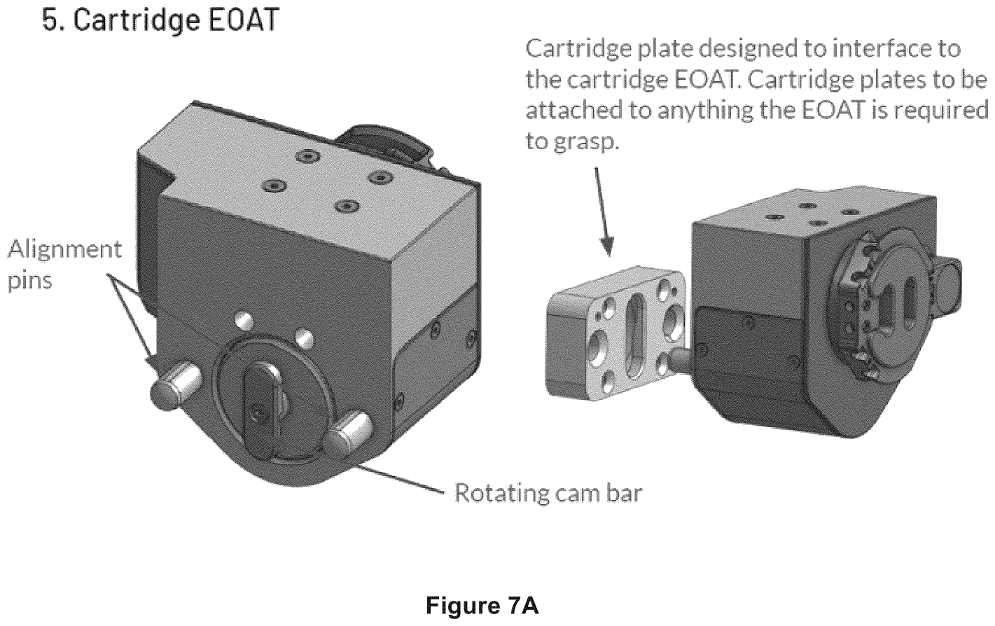

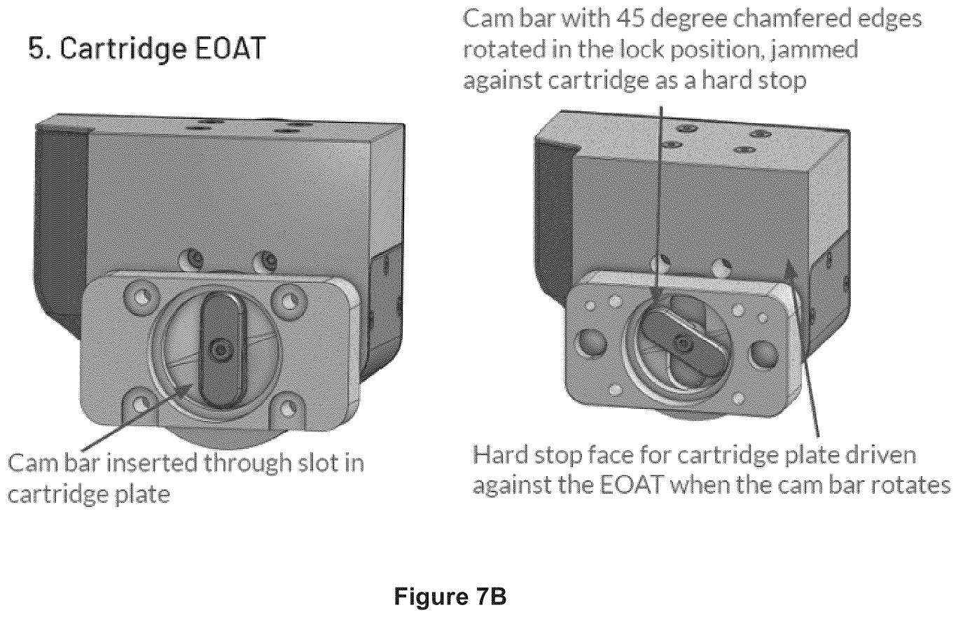

FIGS. 7A-7B illustrate a third exemplary robotic EOAT in accordance with some exemplary embodiments of the present disclosure. In various embodiments, the robotic EOAT includes a novel dual pin and rotating clamp design that rigidly grasps with a single actuator. In some embodiments, the combination of a rotating cam bar with precision alignment pins in a robot EOAT allows for gripping cartridges, trays, cassettes, etc. In some embodiments, there is a distinct performance benefit of this type of align and lock EOAT over traditional pinch grip jaw style EOAT when performing handling tasks on compatible system elements such as cartridges, trays, and cassettes.

In some embodiments, a device is configured for gripping a part. The device includes a robotic EOAT and an interface member. The robotic EOAT includes: a support having a supporting surface that is substantially planar. In some embodiments, the robotic EOAT includes a shaft connected to the support and including a rotational axis substantially perpendicular to the supporting surface of the support. In some embodiments, the robotic EOAT includes a cam bar disposed at an end portion of the shaft. In some embodiments, the cam bar includes (i) a first cam surface facing the supporting surface of the support and characterized by a first cam profile, and (ii) an elongated cross-section in a plane parallel to the supporting surface of the support and having a length larger than a width. In some embodiments, the robotic EOAT includes a plurality of pins connected to the support and positioned around the shaft. In some embodiments, the interface member is configured to be connected to the part. In some embodiments, the interface member includes: a first interface surface that is substantially planar; a second interface surface opposite to the first interface surface, in which when the interface member is connected to the part, the first interface surface faces outwardly and the second interface surface faces the part. In some embodiments, the interface member includes a plurality of pin holes. In some embodiments, each respective pin hole in the plurality of pin holes is configured to receive a corresponding pin in the plurality of pins, thereby facilitating alignment of the robotic EOAT with the interface member. In some embodiments, an elongated slot is formed through the first interface surface and configured in accordance with the elongated cross-section of the cam bar to allow insertion of the cam bar into the interface member. In some embodiments, a blind hole us formed through the second interface surface and aligned with the elongated slot, in which the blind hole has (i) a diameter larger than the length of the elongated cross-section of the cam bar and (ii) a bottom surface within the interface member, thereby allowing the cam bar to rotate once it is inserted into the interface member through the elongated slot. In some such embodiments, rotation of the cam bar causes the first cam surface abut the bottom surface of the blind hole and the first interface surface of the interface member abut the supporting surface of the support, thereby gripping the part by the robotic EOAT through the interface member.

In some embodiments, the part is a cartridge, a tray, or a cassette.

In some embodiments, the first cam profile of the cam bar includes 45 degree chamfered edges, allowing the cam bar to rotate to a lock position and jamming against the bottom surface of the blind hole of the interface member as a hard stop.

In some embodiments, the plurality of pins includes a first pin and a second pin positioned substantially symmetrical to each other with respect to the rotational axis of the shaft. In some embodiments, the plurality of pin holes including a first pin hole and a second pin hole positioned substantially symmetrical to each other with respect to the elongated slot.

In some embodiments, the interface member is in a form of a plate.

In some embodiments, the bottom surface of the blind hole is characterized by a second cam profile.

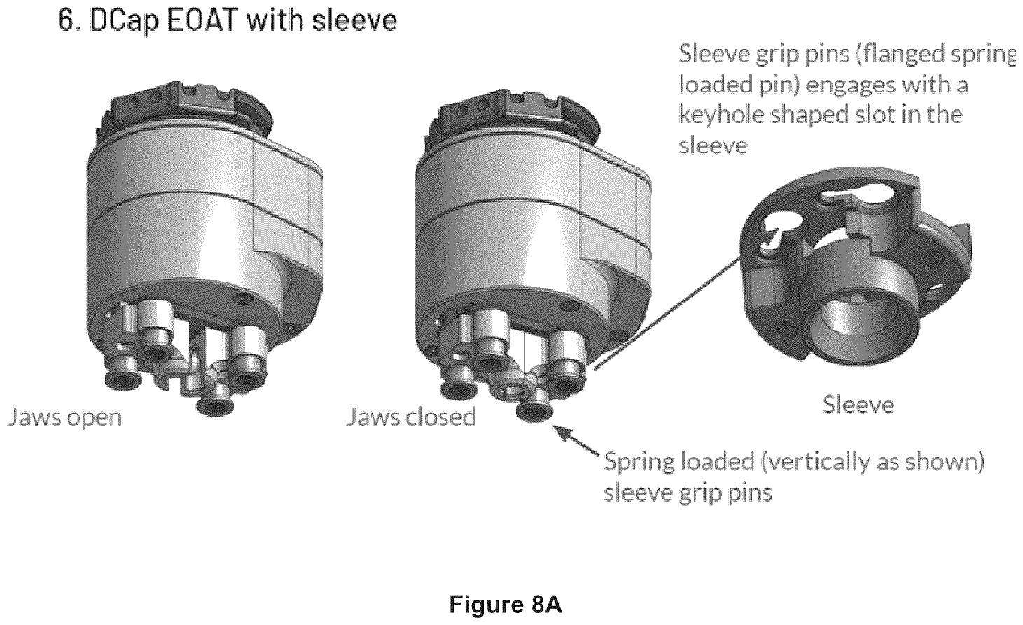

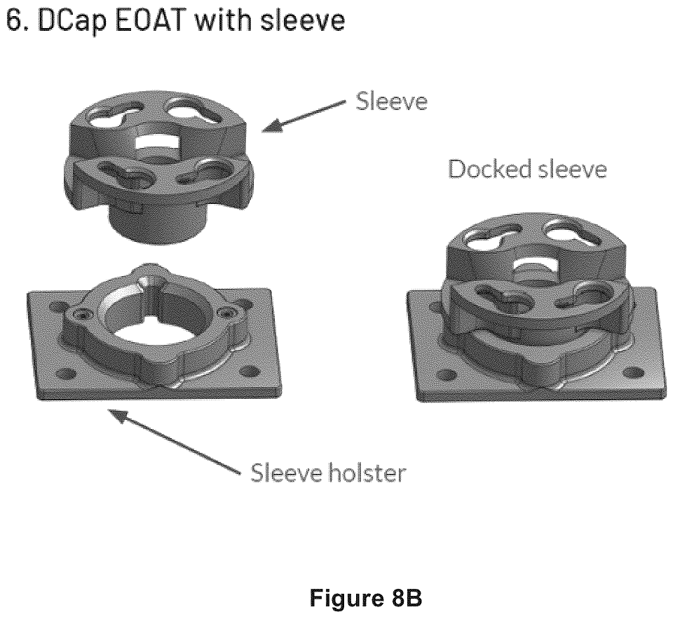

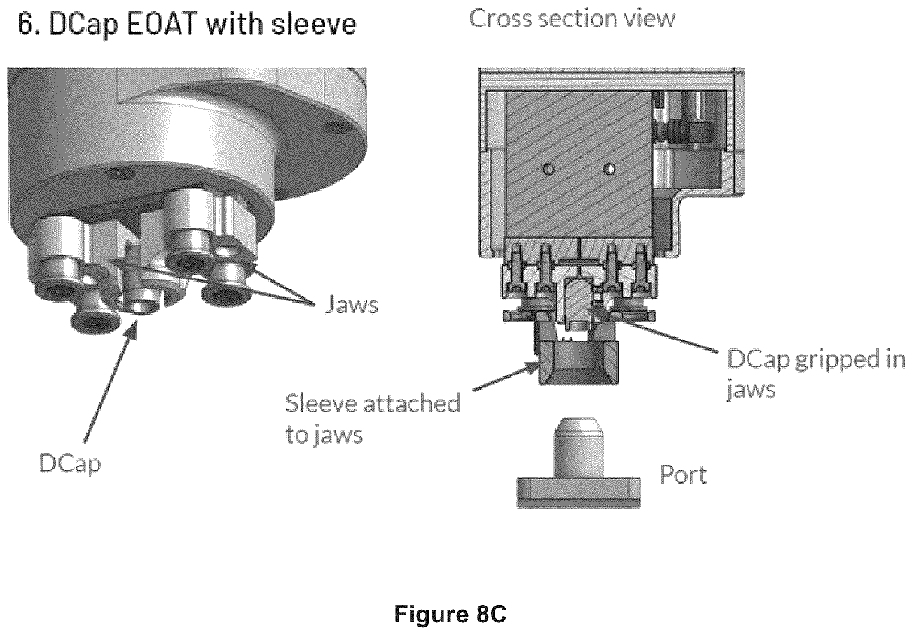

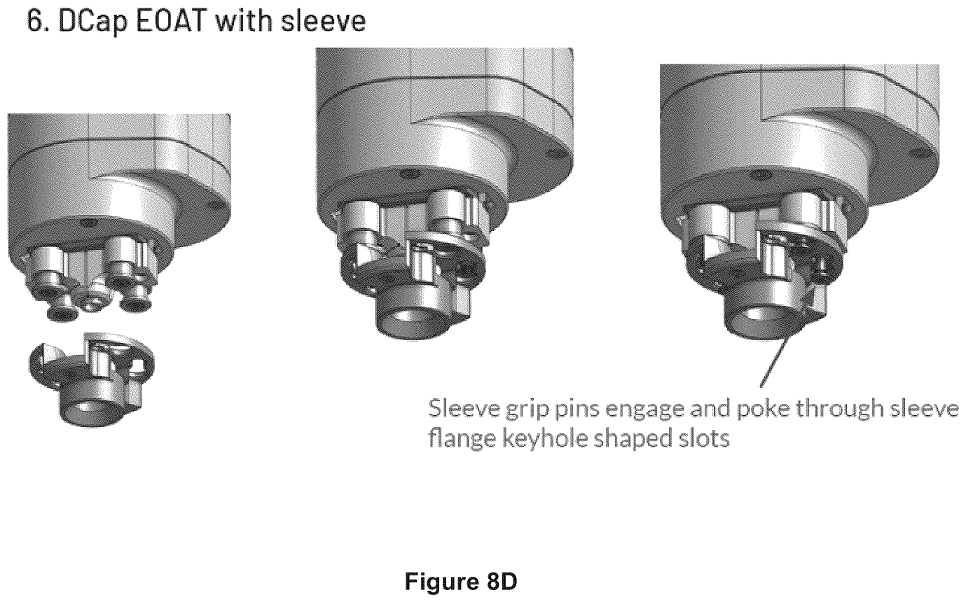

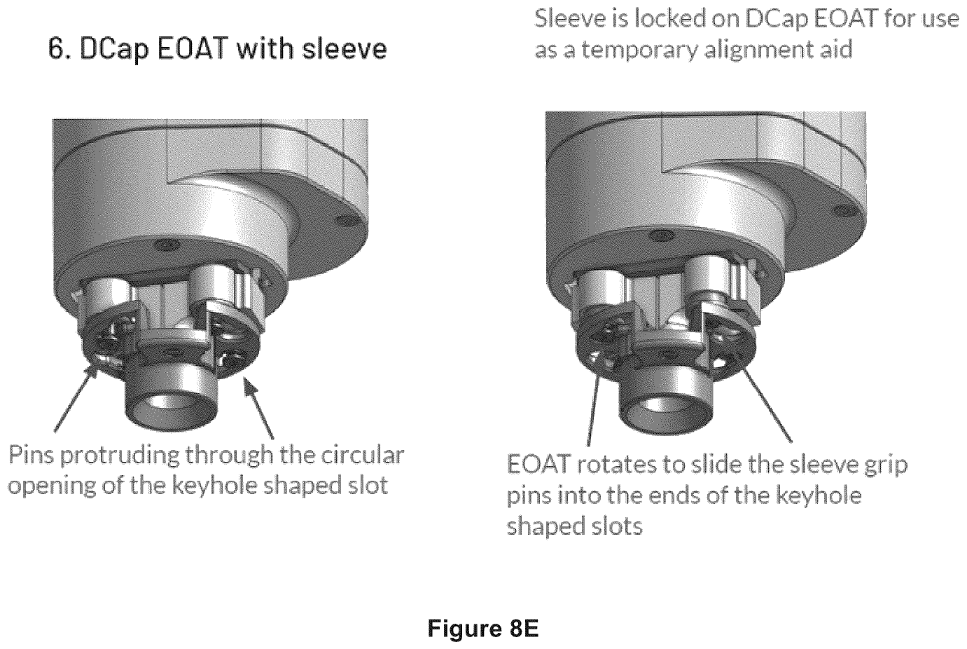

FIGS. 8A-8E illustrate a fourth exemplary robotic EOAT in accordance with some exemplary embodiments of the present disclosure. In various embodiments, the robotic EOAT includes a novel design that uses an external sleeve that mimics a coupler to externally guide the BOAT onto a port feature. One of the key innovations is the coordinated use of the EOAT jaws to both grasp a sanitizing one or more disinfecting caps (DCap) and to also provide grip and locking features to hold an external sleeve that mimics the geometry of a coupler to enable the held DCap to engage with a port utilizing the same alignment aids (chamfer guided floating port body) even though the sanitizing DCap has no twist-on alignment features. In some embodiments, the sleeve is required to be removable because a DCap cannot be picked up by the jaws if the sleeve is installed (height interference with DCap tray).

In some embodiments, a device is configured for coupling a first part with a second part. The device includes a robotic EOAT and a sleeve. In some embodiments, the robotic EOAT includes a support, a first jaw and a second jaw, and a plurality of pins. In some embodiments, the first jaw and the second jaw are connected to the support and operable between an open position and a closed position for gripping and releasing the first part, wherein when in the closed position, the first and second jaws collectively define a first axis. As for the plurality of pins, (i) each of the plurality of pins has an outer flange at a free end portion thereof, (ii) at least one pin in the plurality of pins is disposed at the first jaw; (iii) at least one pin in the plurality of pins is disposed at the second jaw, and (iv) when the first and second jaws are in the closed position, pins in the plurality of pins are positioned around the first axis defined by the first and second jaws at a common radius. In some embodiments, the sleeve includes a rotational axis, a first wall, a plurality of keyhole and a cylindrical section. In some embodiments, the first wall is substantially perpendicular to the rotational axis. In some embodiments, the plurality of keyhole shaped slots is formed on the first wall and corresponding to the plurality of pins of the robotic EOAT. In some such embodiments, each respective slot in the plurality of slots includes (i) a first slot segment larger than the outer flange of a corresponding pin in the plurality of pins and a second slot segment smaller than the outer flange of a corresponding pin, thereby allowing the corresponding pin to insert into the respective slot through the first slot segment and move to the second slot segment to abut the first wall by rotating the sleeve around the rotational axis. In some embodiments, the cylindrical section is connected to or formed with the first wall, in which the cylindrical section is hollow and includes an inner chamfer formed at a free end portion of the cylindrical section to help align the second part with the first part.

In some embodiments, the first part is a DCap.

In some embodiments, the second part is a port.

In some embodiments, the port is a port disclosed herein (e.g., the port disclosed herein with respect to FIGS. 3A-3E).

In some embodiments, each of the plurality of pins is a spring loaded pin.

In some embodiments, each of the plurality of slots is keyhole shaped.

In some embodiments, the first wall includes a first wall segment corresponding to the first jaw and a second wall segment corresponding to the second jaw; and a recess is formed between the first wall segment and the second wall segment.

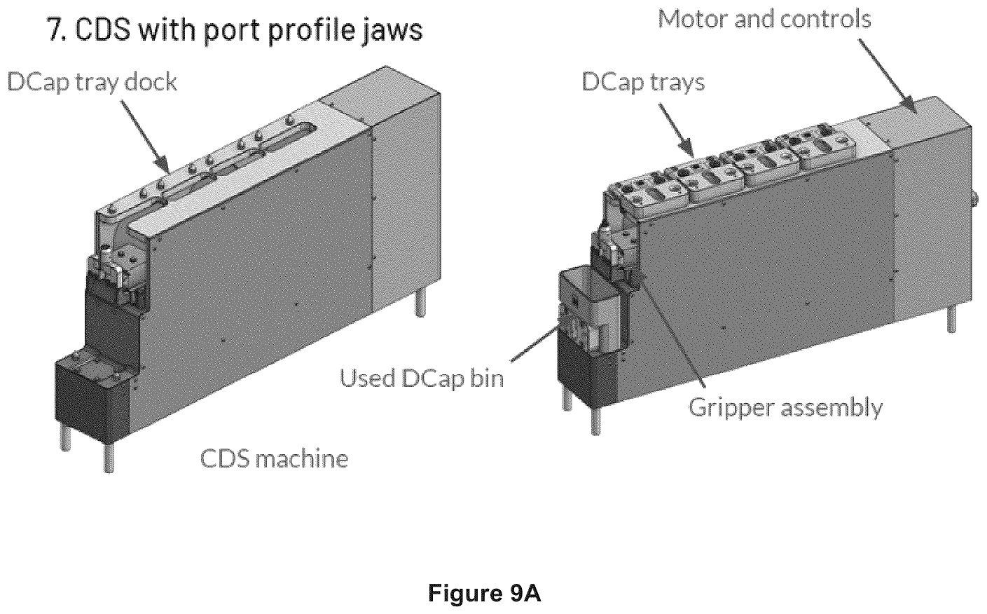

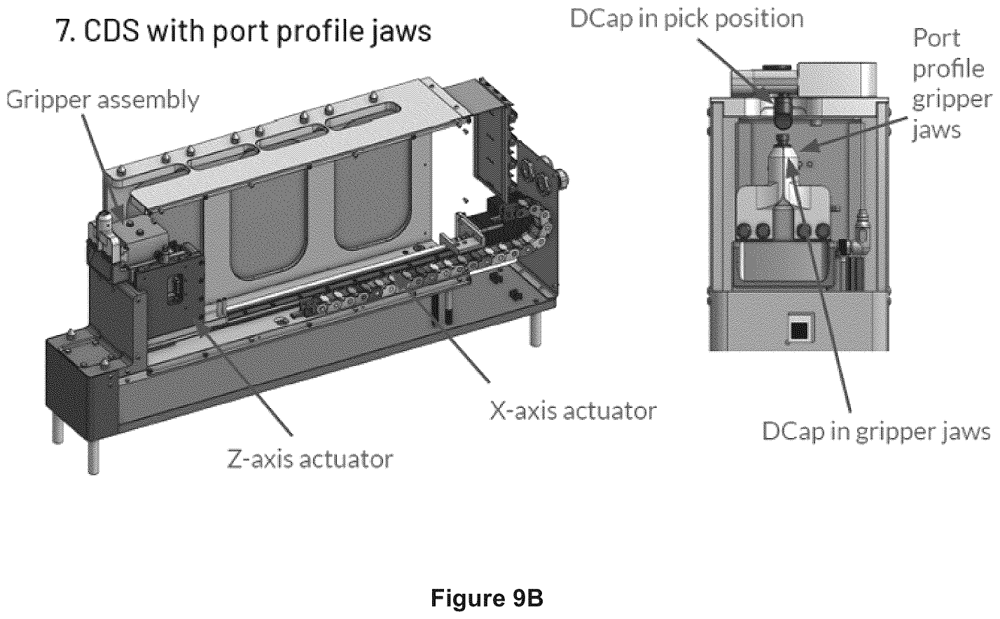

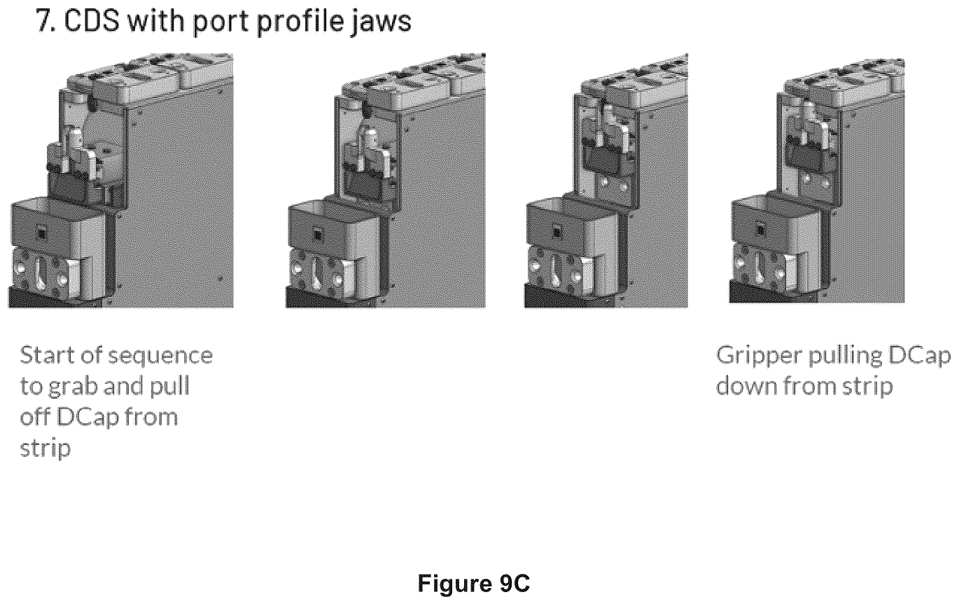

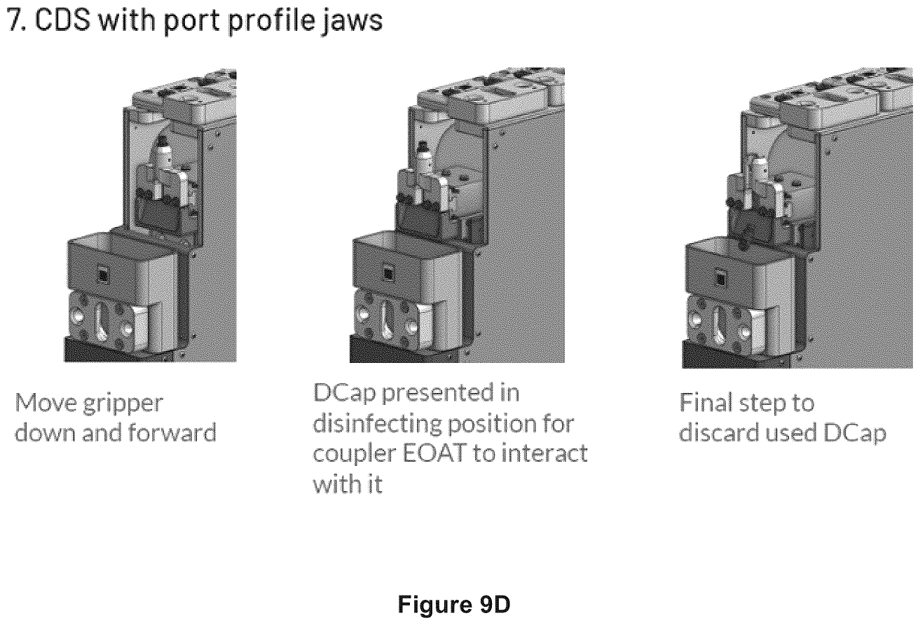

FIGS. 9A-9D illustrate a device for singulating DCaps in accordance with some exemplary embodiments of the present disclosure. In various embodiments, the device is configured to be a unique machine that can singulate DCaps and present them individually in port profile gripper jaws to align with a coupler. One of the key innovations is the combination of mechanized components and structures that enables on demand DCaps mounted on a standard strip to be individually pulled from the strip and presented in gripper jaws that mimic a port external profile to enable a coupler held in a coupler EOAT to engage with the DCap for sanitizing and then the DCap is discarded in a provided waste bin.

In some embodiments, the device is configured for singulating DCaps. The device includes a housing and a gripper assembly. The housing includes an upper wall for mounting a plurality of DCap trays along a first direction of the support, wherein each of the plurality of DCap trays includes one or more DCaps exposed to a space below the upper wall. The gripper assembly is disposed below the upper wall of the housing and movable along the first direction and a vertical direction substantially perpendicular to the first direction, wherein the gripper assembly includes a first jaw and a second jaw operable between an open position and a closed position for gripping and releasing a DCap.

In some embodiments, the device further includes: a first actuator to move the gripper assembly in the first direction; and a second actuator to move the gripper assembly in the vertical direction.

In some embodiments, the device further includes a bin disposed below the first and second jaws of the gripper assembly to receive a used DCap.

In some embodiments, the gripper assembly is operated to: (i) move in the first direction to position the first and second jaws under a first DCap; (ii) move upward in the vertical direction to position the first and second jaws at a level corresponding to the first DCap; (iii) close the first and second jaws to grip the first DCap; (iv) move downward in the vertical direction to pull off the first DCap; (v) move in the first direction to position the first DCap at a location for sanitizing a part; and (vi) optionally or additionally, open the first and second jaws to release the first DCap.

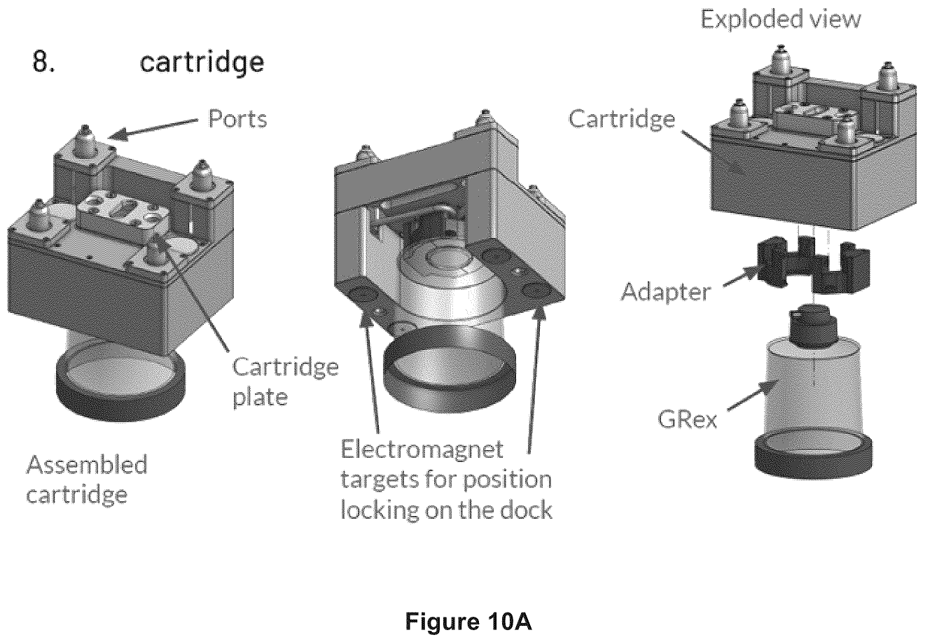

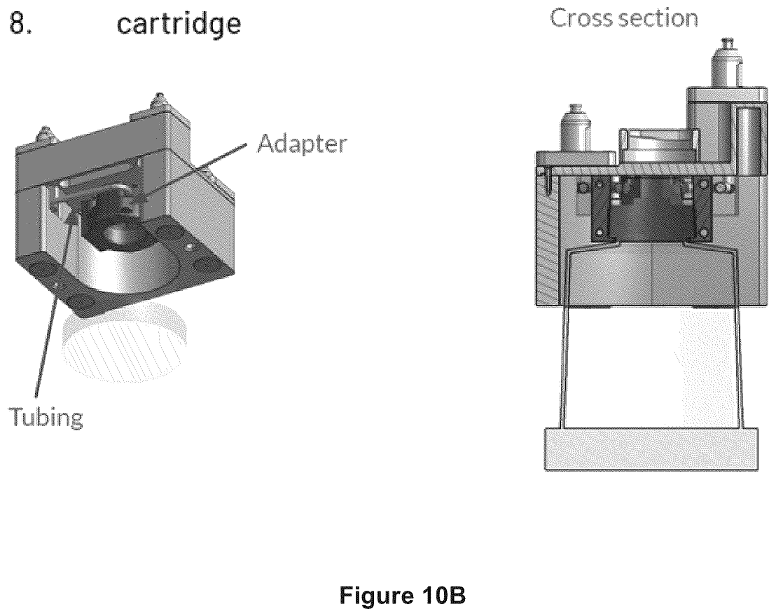

FIGS. 10A-10B illustrate a first exemplary cartridge in accordance with some exemplary embodiments of the present disclosure. In various embodiments, the cartridge includes a novel design that converts a standard vessel to a robot compatible cartridge. One of the key innovations is taking an industry standard bioreactor and adapting it to a robot compatible compact cartridge with gripping features, floating ports, concealed tubing, and docking features.

In some embodiments, the cartridge is configured for converting a first bioreactor, which includes a cap and a plurality of bioreactor ports, to a robot-operable bioreactor. The cartridge includes a cartridge body including a side wall, an upper wall connected to or formed with an upper portion of the side wall, and an interior space defined by the side wall and the upper wall. The cartridge also includes an adapter disposed in the interior space and connected to the cartridge body, wherein the adapter is configured for coupling with the cap of the first bioreactor, thereby connecting the first bioreactor to the cartridge body. The cartridge further includes a plurality of cartridge ports, each disposed at the side or upper wall of the cartridge body. Furthermore, the cartridge includes a plurality of connectors, each housed by a corresponding cartridge port in the plurality of cartridge ports and including a first end outside of the cartridge body and a second end inside of the cartridge body. In addition, the cartridge includes a plurality of tubes disposed in the interior space of the cartridge body, each fluidly connecting a corresponding bioreactor port in the plurality of bioreactor ports with a second end of a corresponding connector in the plurality of connectors.

In some embodiments, the first bioreactor is a Gas Permeable Rapid Expansion bioreactor (G-Rex®).

In some embodiments, the plurality of bioreactor ports is formed at the cap of the first bioreactor.

In some embodiments, the adapter includes a plurality of slots for accommodating the plurality of bioreactor ports, the plurality of tubes, or any combination thereof.

In some embodiments, each of the plurality of cartridge ports is a port disclosed herein (e.g., the port disclosed herein with respect to FIGS. 3A-3E).

In some embodiments, the plurality of cartridge ports is disposed at the upper wall of the cartridge body.

In some embodiments, the upper wall of the cartridge body includes a first level and a second level above the first level, and at least one cartridge port in the plurality of cartridge ports is disposed at each of the first and second levels of the upper wall.

In an exemplary embodiment, two cartridge ports in the plurality of cartridge ports are disposed at each of the first and second levels of the upper wall.

In some embodiments, the cartridge further includes a cartridge plate disposed at the side or upper wall of the cartridge body and configured to be grasped by a robotic EOAT.

In an exemplary embodiment, the cartridge plate is disposed at a middle portion of the upper wall of the cartridge body.

In some embodiments, a circular hole is formed on a side of the cartridge plate that faces the cartridge body; and an elongated slot is formed on an opposite side of the cartridge plate and to the circular hole, wherein the elongated slot is concentric with the circular hole and has a length equal to or less than a diameter of the circular hole, thereby allowing a cam bar of a robotic EOAT to insert through the elongated slot into the circular hole and rotate to engage with the cartridge plate.

In some embodiments, the cartridge further includes a plurality of pin hole formed at a lower portion of the side wall of the cartridge body to facilitate alignment and positioning of the cartridge on a dock.

In some embodiments, the cartridge further includes a plurality of electromagnet targets disposed at a lower portion of the side wall of the cartridge body to facilitate positioning and locking of the cartridge on a dock.

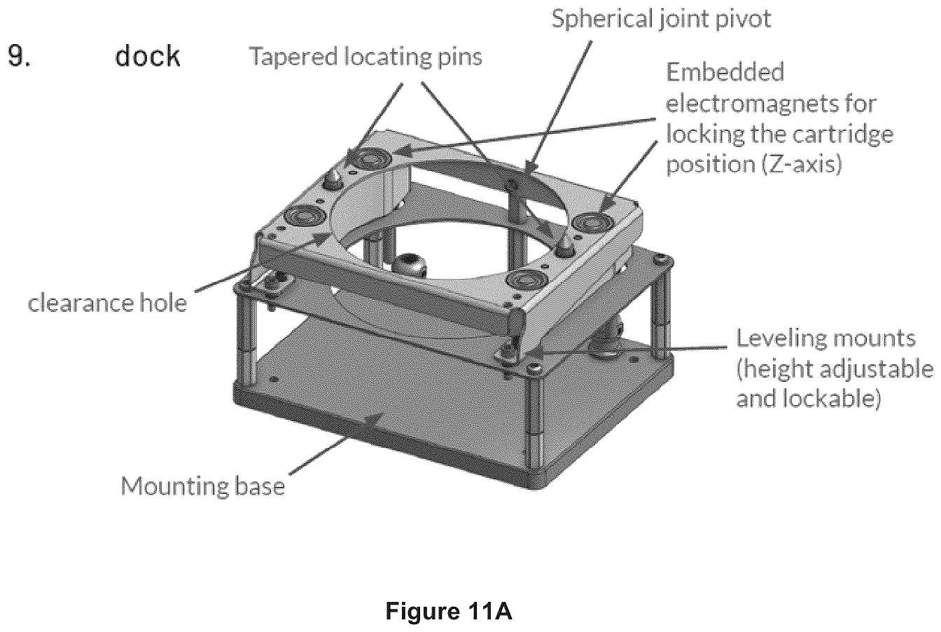

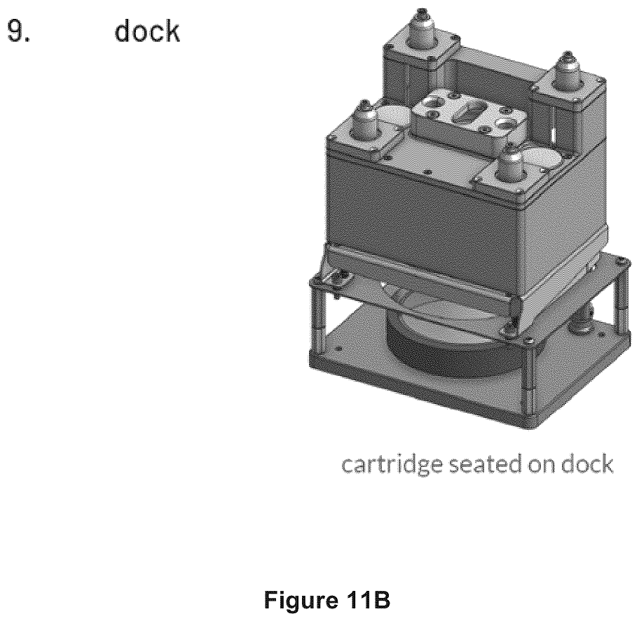

FIGS. 11A-11B illustrate a first exemplary dock in accordance with some exemplary embodiments of the present disclosure. In various embodiments, the dock includes a novel design that incorporates leveling features, electromagnet locking features and/or other features. One of the key innovations is a combination of locating and locking features that work in conjunction with the cartridge design to enable a standard bioreactor have repeatable positioning capability, robot compatible gripping and handling, and position locking capabilities to allow couplers to be twisted on and off the cartridge ports.

In some embodiments, the dock is configured for mounting a cartridge. The dock includes a mounting base, a first mounting member, a second mounting member, a plurality of pins, and a plurality of electromagnets. The first mounting member is connected to the mounting base and spaced apart from the mounting base in a first direction, wherein the first mounting member includes a first through-hole. The second mounting member is connected to the first mounting member by a plurality of leveling mounts, wherein (i) each of the plurality of leveling mounts has a height that is adjustable and lockable, and (ii) the second mounting member includes a second through-hole concentric with the first through-hole of the first mounting member to accommodate a vessel of the cartridge. The plurality of pins is disposed at the second mounting member and corresponds to a plurality of pin holes of the cartridge to facilitate positioning and locking of the cartridge on the dock. The plurality of electromagnets is disposed at the second mounting member and corresponds to a plurality of electromagnet targets of the cartridge to facilitate positioning and locking of the cartridge on the dock.

In some embodiments, the cartridge is a cartridge disclosed herein (e.g., the cartridge disclosed herein with respect to FIGS. 10A-10B).

In some embodiments, each of the plurality of pins is tapered.

In some embodiments, the second through-hole is a clearance hole.

In some embodiments, adjusting the heights of the plurality of leveling mounts allows the vessel of the cartridge to sit on the mounting base.

In some embodiments, the dock further includes one or more spherical joint pivots.

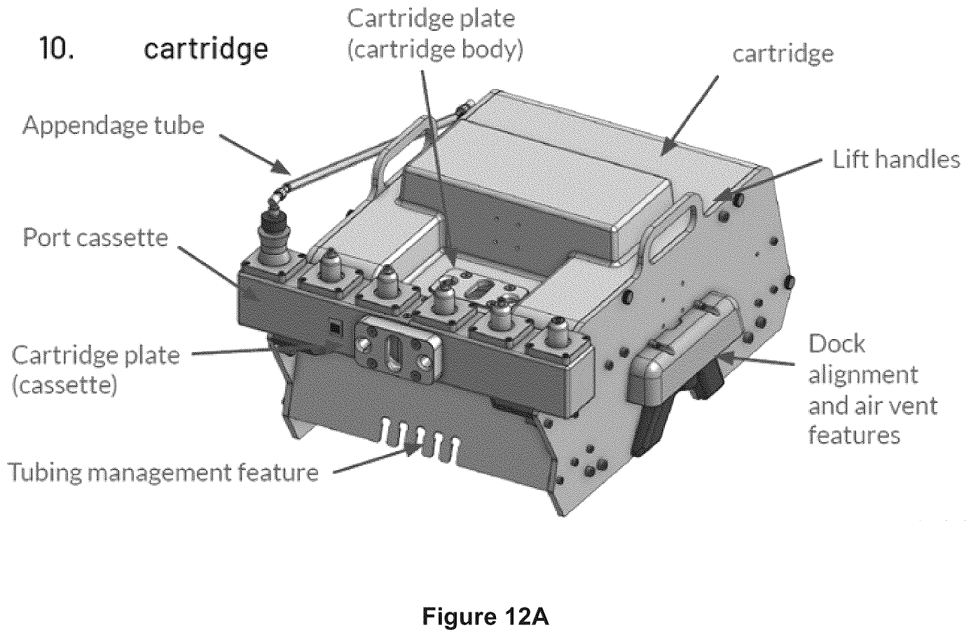

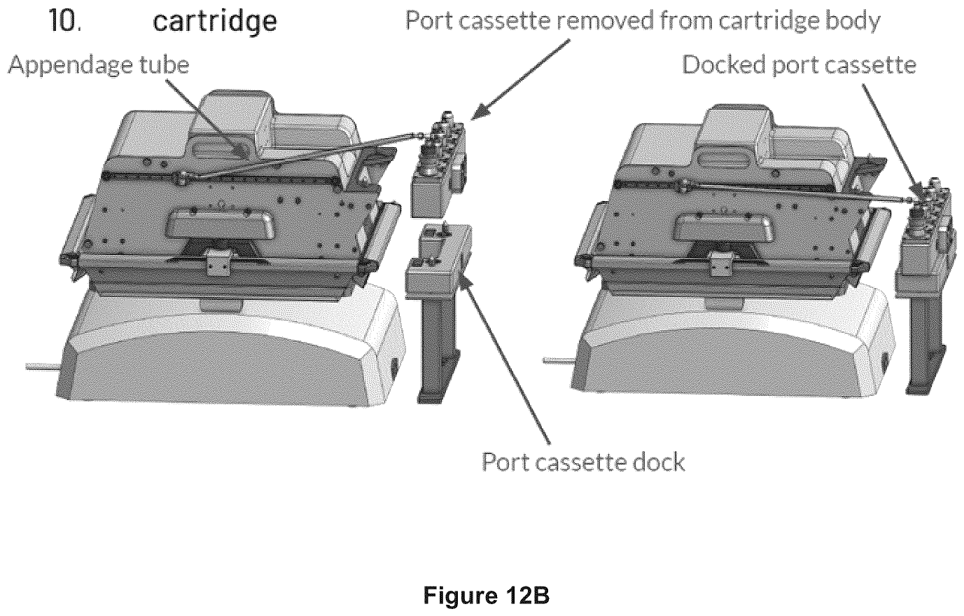

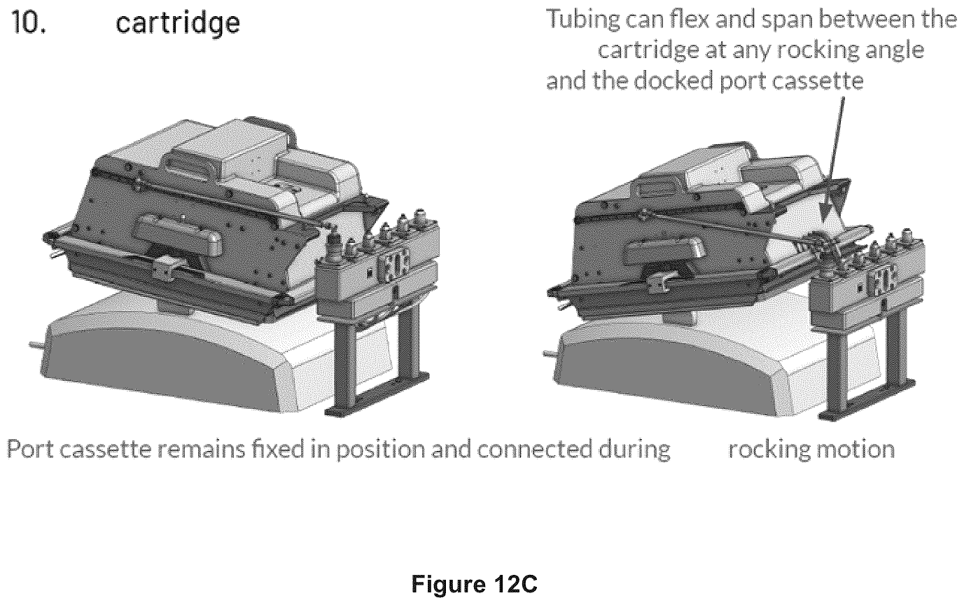

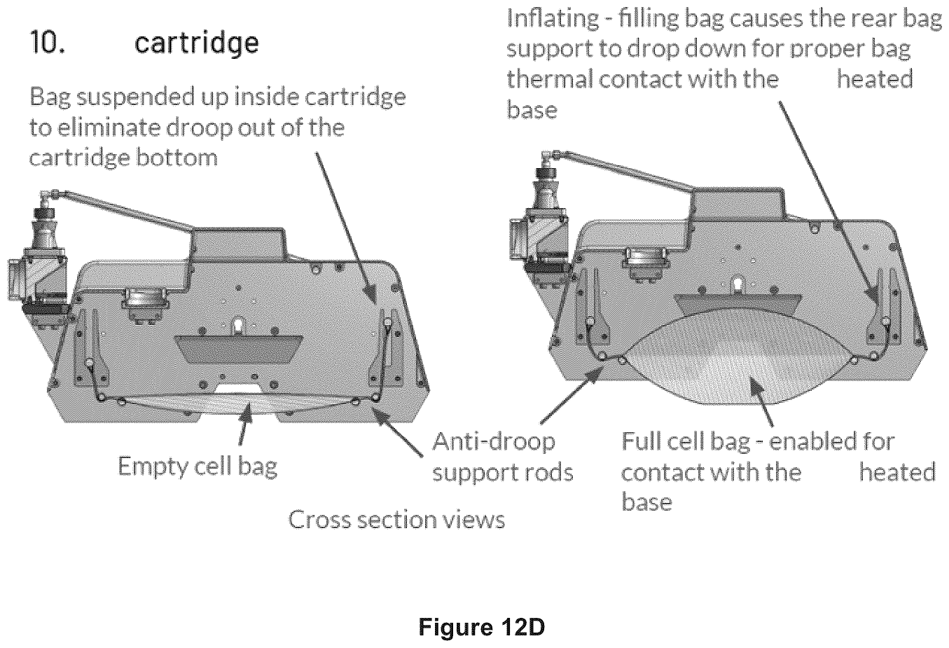

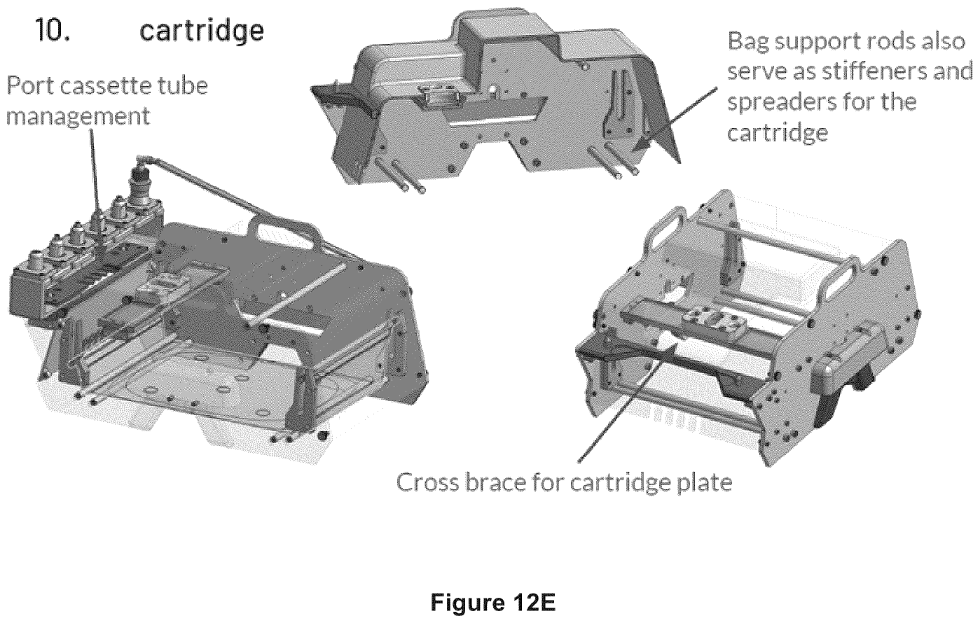

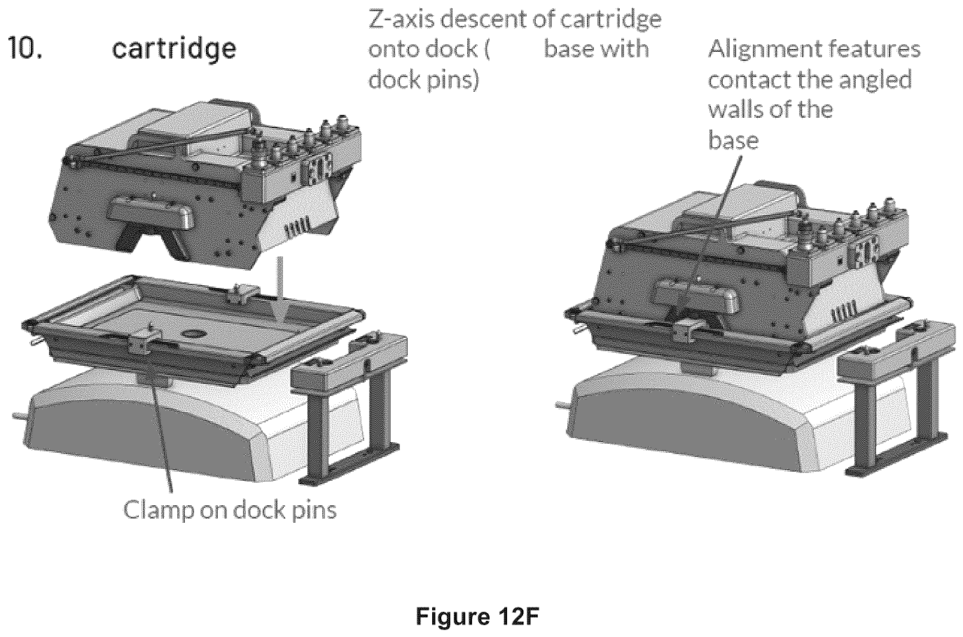

FIGS. 12A-12F illustrate a second exemplary cartridge in accordance with some exemplary embodiments of the present disclosure. The cartridge includes a novel design that mimics a standard cover assembly with special robot compatible port features, bag support system, and docking features. One of the key innovations is a series of design modifications to the original protective cover to create a robot compatible cartridge with updated manufacturing concepts, removable/decoupled and dockable port cassette, sliding appendage tube, multi-function docking features, anti-droop bag mounts, integral tubing management, and standard robot grip features.

In some embodiments, the cartridge includes a cartridge body, a bag, a first interface member, a port cassette, a second interface member, and an appendage tube. The cartridge body includes an interior space and a first port on a wall. The bag is disposed in the interior space and in fluid communication with the first port. The first interface member is connected to the cartridge body and configured to be gripped by a robotic EOAT, thereby facilitating moving of the cartridge body and docking of the cartridge body on a cartridge dock. The port cassette includes a mounting member removably coupled with the cartridge body and a plurality of second ports disposed at the mounting member. The second interface member is connected to the mounting member of the port cassette and configured to be gripped by the robotic EOAT, thereby facilitating removal of the port cassette from the cartridge body and docking of the port cassette on a cassette dock. The appendage tube is configured for fluidly connecting the first port of the cartridge body with a second port in the plurality of second ports disposed at the mounting member of the cassette.

In some embodiments, the bag is a bioreactor.

In some embodiments, a second port in at least a subset of the plurality of second parts is a port disclosed herein (e.g., the port disclosed herein with respect to FIGS. 3A-3E).

In some embodiments, the appendage tube is able to flex and span between the cartridge body at any rocking angle and the docked port cassette.

In some embodiments, the cartridge further includes a plurality of support rods connected to the cartridge body and configured for suspending the bag inside the cartridge body when it is empty and preventing the empty bag from drooping out of a bottom of the cartridge body.

In some embodiments, the bag when filled inflates and causes a bag support to drop down, thereby allowing the filled bag to be in thermal contact with a heat plate of the cartridge dock.

In some embodiments, each of the cartridge body and the port cassette includes a tube management unit.

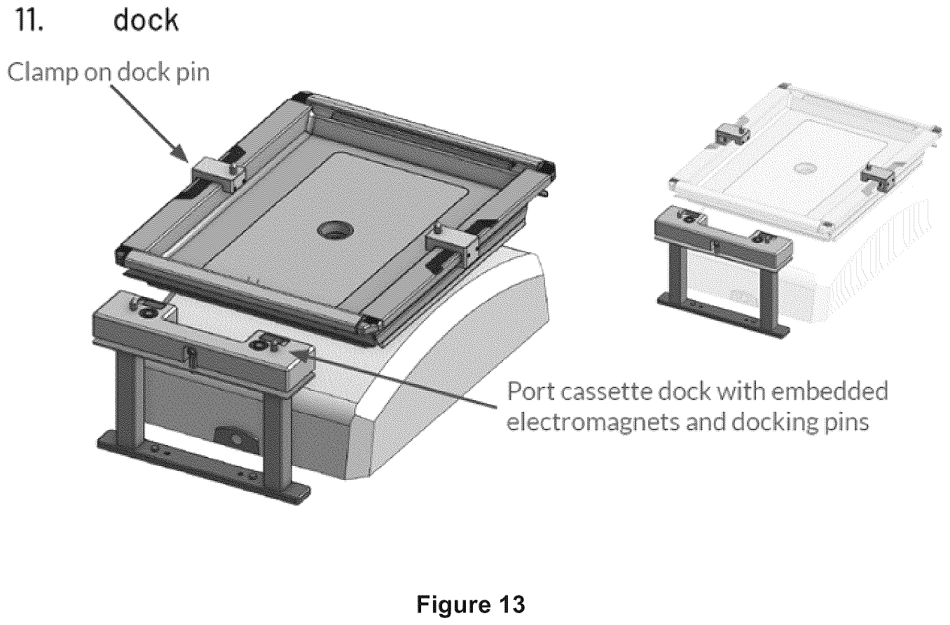

FIG. 13 illustrates a second exemplary dock in accordance with some exemplary embodiments of the present disclosure. In various embodiments, the dock includes a unique docking pin design that clamps onto a standard heat plate without any modifications to the. [symbol] One of the key innovations is a combination of using the existing base profile as a perimeter chamfer to guide the cartridge into position and clamp on docking pins that involve no modification to the base to install.

In some embodiments, the dock includes a cartridge dock, a cassette dock, or both of the cartridge dock and the cassette dock. The cartridge dock includes a base and a plurality of clamps. The base includes a perimeter edge, wherein at least a portion of the perimeter edge is chamfered to guide a cartridge into position. Each of the plurality of clamps is coupled with the perimeter edge of the base and includes a first pin to couple with a pin hole of the cartridge. The cassette dock includes a stand having an upper wall, a plurality of second pins, and a plurality of electromagnets. The plurality of second pins is disposed at the upper wall and corresponds to a plurality of pin holes of a cassette to facilitate positioning and locking of the cassette on the cassette dock. The plurality of electromagnets is disposed at the upper wall and corresponds to a plurality of electromagnet targets of the cassette to facilitate positioning and locking of the cassette on the cassette dock.

In some embodiments, the cartridge dock and the cassette dock are independent from each other.

In some embodiments, each of the plurality of clamps is movable in one or more directions with respect to the base.

In some embodiments, the cartridge dock further includes a heat plate disposed at the base.

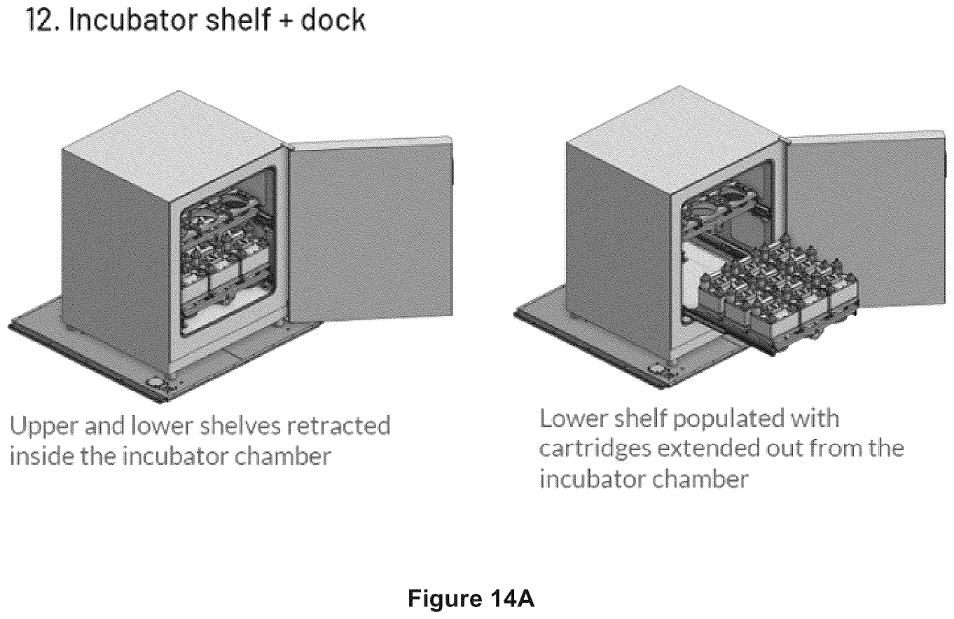

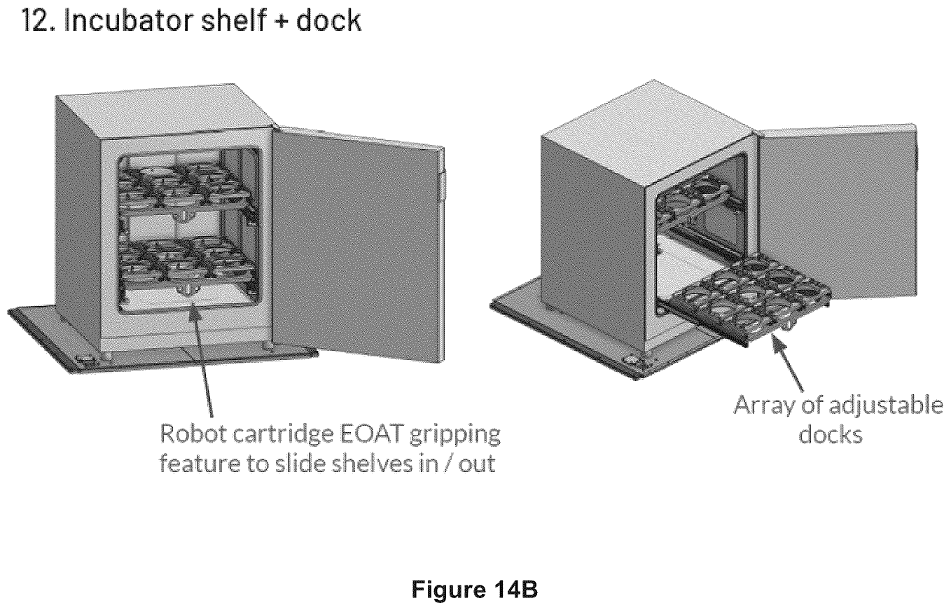

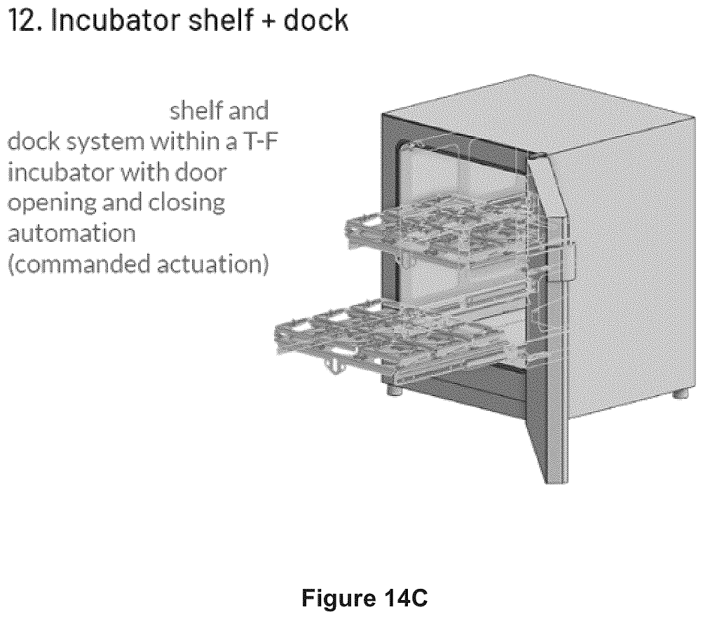

FIGS. 14A-14C illustrate an exemplary incubator in accordance with some exemplary embodiments of the present disclosure. In various embodiments, the incubator includes a custom drop-in incubator slide out shelf design that utilizes the existing mounting features into the incubator providing an ultra-high density array of docks with individual leveling features. One of the key innovations is a combination of features for easy installation into an unmodified T-F incubator, high temperature SS drawer slides, custom shelf assembly with a high density array of docks producing a robot accessible incubator system via robot actuated slide out shelves.

In some embodiments, an accessory for an incubator includes one or more slides and a shelf. Each of the one or more slides includes an outer member, an intermediate member and an inner member, wherein the outer member is configured to be connected to a wall or a rack of the incubator, the intermediate member is slidable relative to the outer member, and the inner member is slidable relative to the intermediate member. The shelf is connected to the inner member of each of the one or more slides, and including an interface member at a front side thereof, wherein the interface member is configured to be gripped by a robotic EOAT for pulling at least a portion of the shelf out of the incubator or pushing the shelf into the incubator.

In some embodiments, the incubator includes a door operable by command.

In some embodiments, the accessory further includes an array of docks disposed on an upper side of the shelf, each configured for holding a cartridge.

In some embodiments, the array of docks is one-dimensional or two-dimensional array.

In some embodiments, at least two docks in the array of docks are different than each other.

In some embodiments, at least two docks in the array of docks are substantially identical to each other.

In some embodiments, a dock in the array of docks is a dock disclosed herein (e.g., the dock disclosed herein with respect to FIGS. 11A-11B).

In some embodiments, the intermediate member is slidable relative to the outer member and the inner member is slidable relative to the intermediate member without using lubrication.

In some embodiments, the outer, intermediate and inner members of each of the one or more slides are made of stainless steel.

In some embodiments, the one or more slides include a first slide disposed at a first side of the incubator and a second slide disposed at a second side of the incubator.

In some embodiments, the robotic EOAT includes a support having a supporting surface that is substantially planar; a shaft connected to the support and including a rotational axis substantially perpendicular to the supporting surface of the support; a cam bar disposed at an end portion of the shaft, wherein the cam bar includes (i) a first cam surface facing the supporting surface of the support and characterized by a first cam profile, and (ii) an elongated cross-section in a plane parallel to the supporting surface of the support and having a length larger than a width; and a plurality of pins connected to the support and positioned around the shaft. The interface member includes: a first interface surface facing outwardly, wherein the first interface surface is substantially planar; a second interface surface opposite to the first interface surface; a plurality of pin holes, wherein each respective pin hole in the plurality of pin holes is configured to receive a corresponding pin in the plurality of pins, thereby facilitating alignment of the robotic EOAT with the interface member; an elongated slot configured in accordance with the elongated cross-section of the cam bar to allow insertion of the cam bar and rotation of the cam bar after passing through the elongated slot. In some such embodiments, rotation of the cam bar causes the first cam surface abut the second interface surface of the interface member and the first interface surface of the interface member abut the supporting surface of the support, thereby gripping the interface member.

In some embodiments, the interface member is shaped in a form of a lug; and the plurality of pin holes includes a first pin hole and a second pin hole positioned substantially symmetrical to each other with respect to the elongated slot.

In some embodiments, the shelf includes: a substantially planar top sheet having a front edge; and a front sheet extended from the front edge of the substantially planar top sheet at an angle and with a crease, wherein the engaging member is a portion of the front sheet.

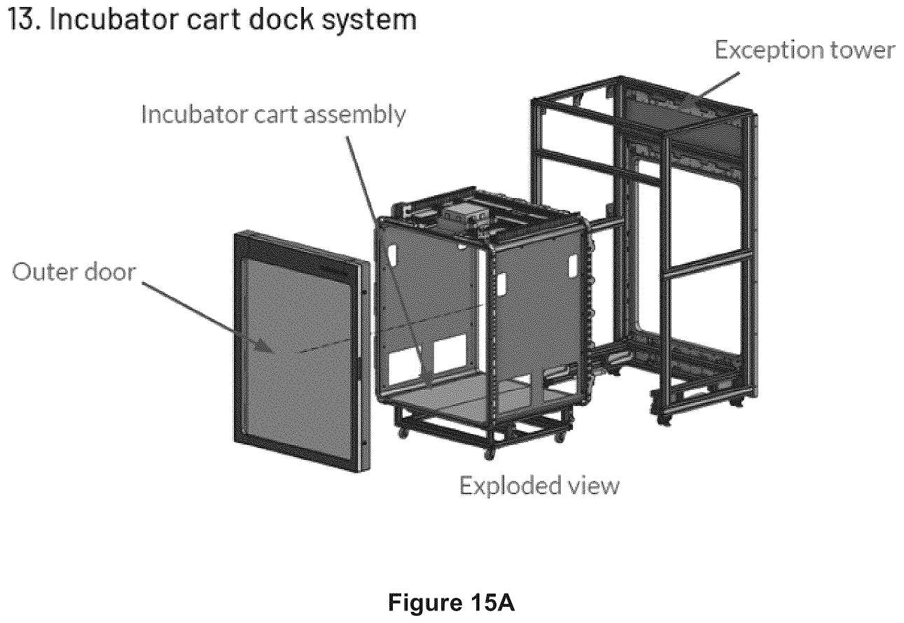

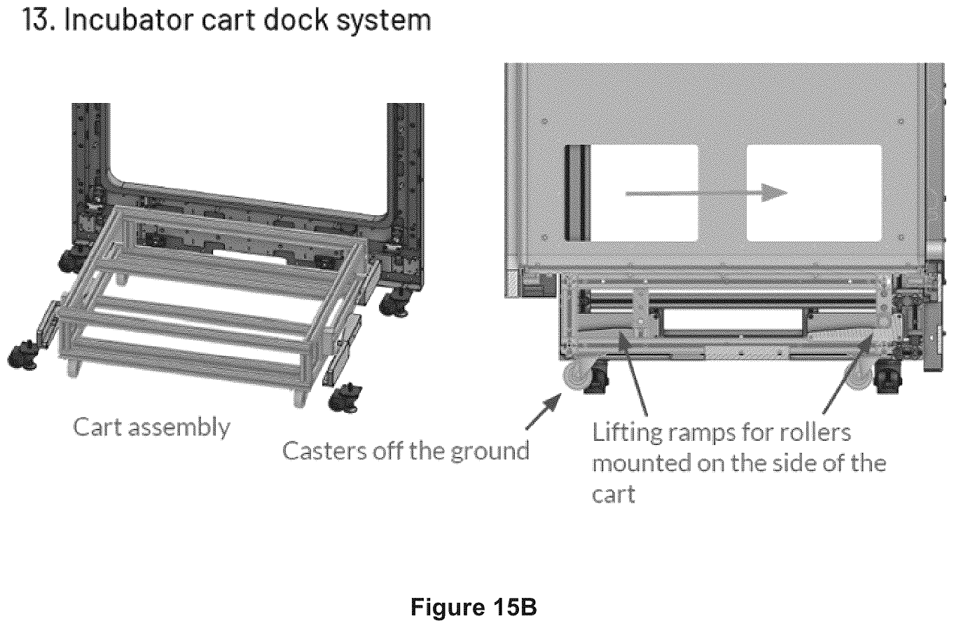

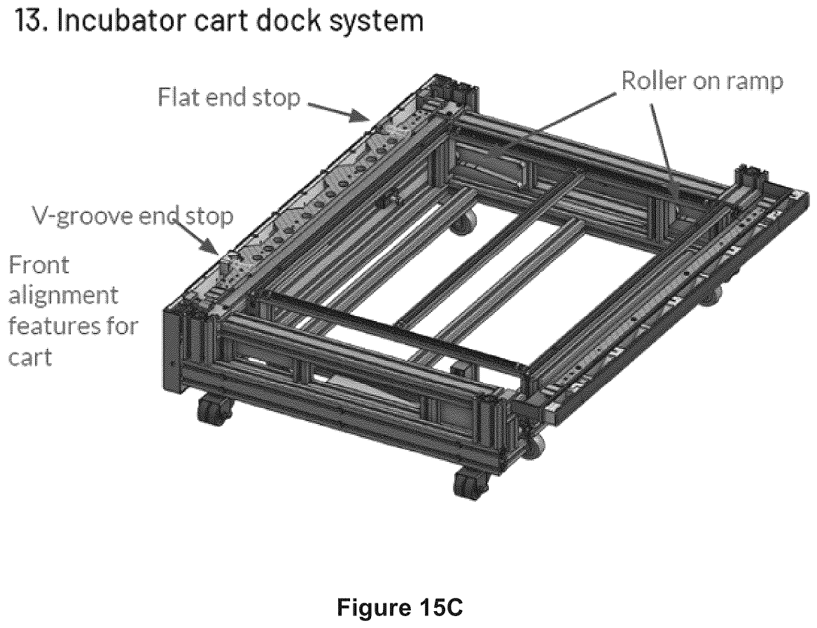

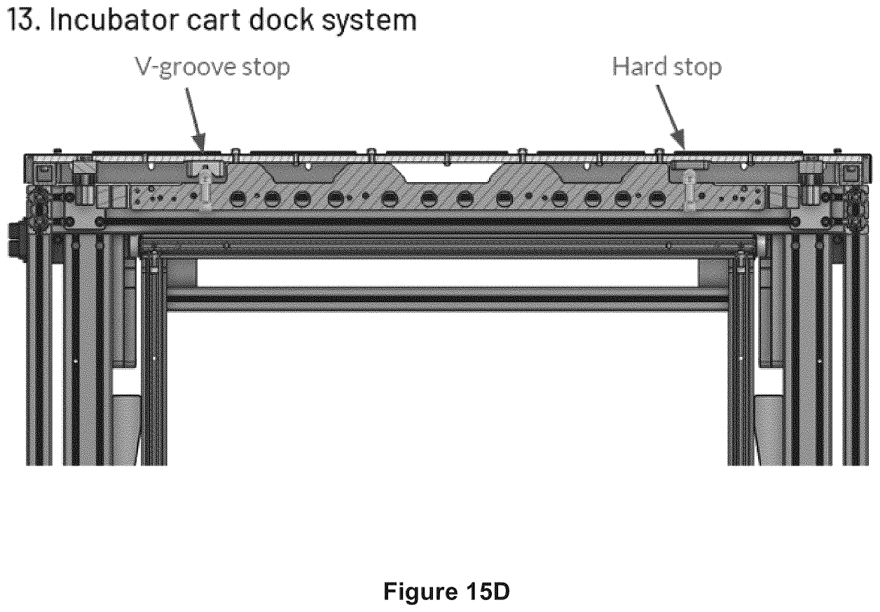

FIGS. 15A-15D illustrate an exemplary cart dock system in accordance with some exemplary embodiments of the present disclosure. In various embodiments, the dock system includes a novel exception tower design with a roll-in/roll-out cart for the incubator that maintains repeatable alignment when locked into position. One of the key innovations is the mounting of the incubator on a wheeled cart that when pushed into the install position rides up on a series of ramps to lift the wheels off the floor and dock the cart against alignment hard stops.

In some embodiments, the dock system includes a tower and a cart. The tower includes one or more ramp sets, wherein each of the one or more ramp sets includes a first ramp at a first side of the tower and a second ramp at a second side of the tower. The cart includes: a plurality of casters at a bottom of the cart; and one or more roller sets, wherein each of the one or more roller sets includes a first roller at a first side of the cart and a second roller at a second side of the cart. In some such embodiments, (i) each respective roller set in the one or more roller sets is associated with a corresponding ramp set in the one or more ramp sets, (ii) the first roller of the respective roller set rolls on the first ramp of the corresponding ramp set, and (iii) the second roller of the respective roller set rolls on the second ramp of the corresponding ramp set, thereby lifting the casters off a ground.

In some embodiments, the one or more ramp sets includes a plurality of ramp sets, and the one or more roller sets includes a plurality of roller sets.

In some embodiments, the dock system further includes one or more end stops disposed at the tower or the cart.

In an exemplary embodiment, the one or more end stops includes a flat end stop, a V-groove end stop, or both.

In some embodiments, the dock system further includes one or more front alignment features for aligning the cart.

In some embodiments, the dock system further includes an outer door.

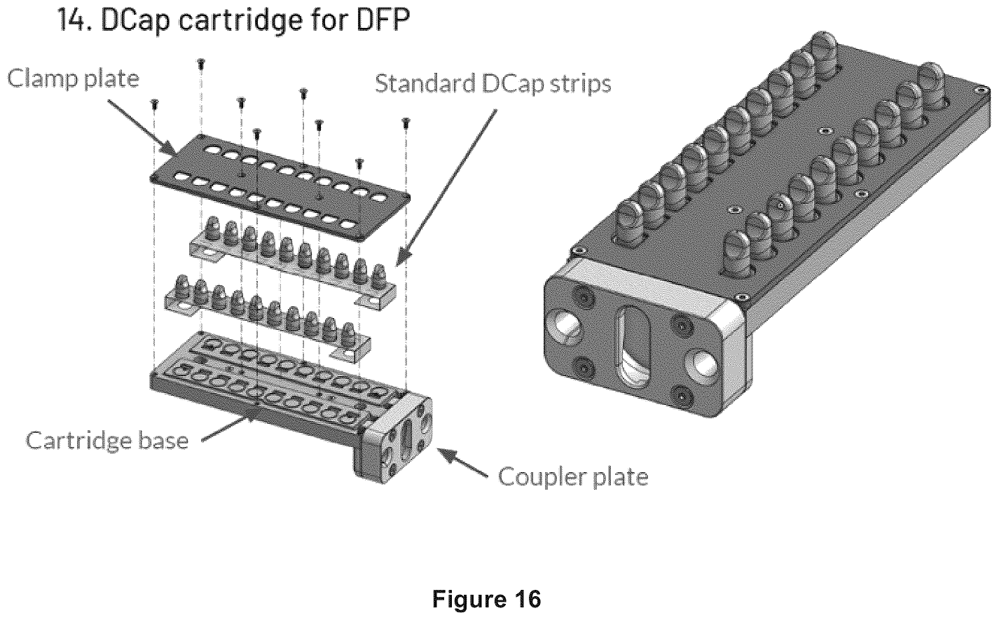

FIG. 16 illustrates a third exemplary cartridge in accordance with some exemplary embodiments of the present disclosure. In various embodiments, the cartridge includes a custom-easy-to-load cartridge design that aligns and secures standard DCap strips for robot handling. One of the key innovations is the cartridge locking feature design that holds a standard strip of adhered DCaps.

In some embodiments, a DCap cartridge includes a cartridge base, a clamp plate, one or more strips, and an interface member. The clamp plate is connected to the cartridge base at a first side of the cartridge base, wherein the clamp plate includes an array of holes. The one or more strips are disposed between the cartridge base and the clamp plate and secured on the cartridge base by the clamp plate, wherein each respective strip in the one or more strips includes a corresponding plurality of DCaps, each DCap passing through a corresponding hole in the array of holes of the clamp plate. The interface member is connected to the cartridge base at a second side of the cartridge base and configured to be gripped by a robotic EOAT, thereby facilitating moving of the cartridge and docking of the cartridge on a cartridge dock.

In some embodiments, the array of holes includes a plurality of rows of holes.

In an exemplary embodiment, the number of the strips is less than or equal to the number of rows of holes of the clamp plate.

In some embodiments, each of the one or more strips is a standard DCap strip.

In some embodiments, the first side of the cartridge base includes one or more raisers, and each raiser is patterned to help align and/or secure a strip in the one or more strips.

In some embodiments, the interface member is in a form of a plate and is substantially perpendicular to the cartridge base.

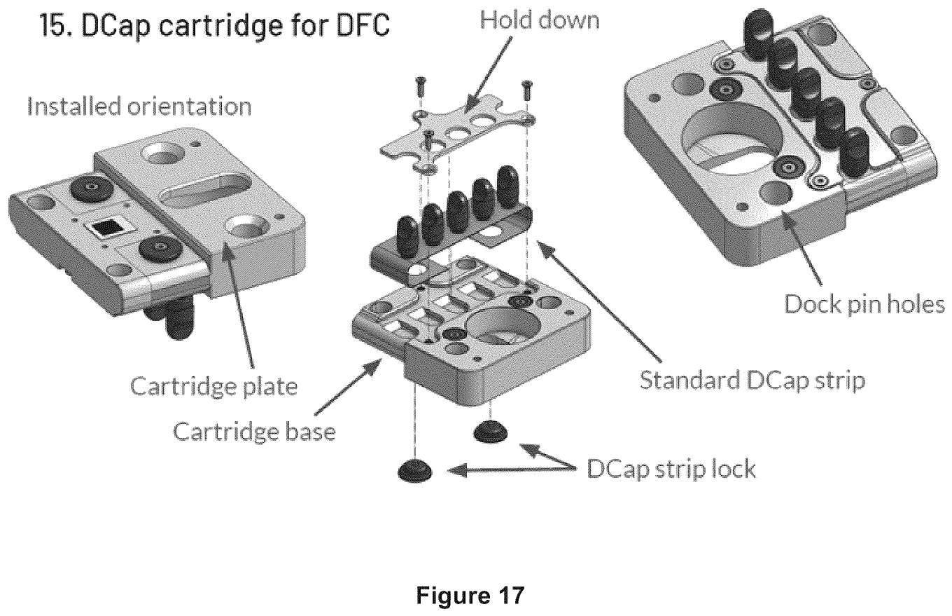

FIG. 17 illustrates a fourth exemplary cartridge in accordance with some exemplary embodiments of the present disclosure. In various embodiments, the cartridge includes custom-easy-to-load cartridge design that aligns and secures standard DCap strips for robot handling. One of the key innovations is the cartridge locking feature design which holds a standard strip of adhered DCaps.

In some embodiments, a DCap cartridge includes a cartridge base, a clamp plate, a strip, and an interface member. The clamp plate is connected to the cartridge base at a first side of the cartridge base, wherein the clamp plate includes a row of holes. The strip is disposed between the cartridge base and the clamp plate and secured on the cartridge base by the clamp plate, wherein the strip includes a plurality of DCaps, each DCap passing through a corresponding hole in the row of holes of the clamp plate. The interface member is connected to the cartridge base at a second side of the cartridge base and configured to be gripped by a robotic EOAT, thereby facilitating moving of the cartridge and docking of the cartridge on a cartridge dock.

In some embodiments, the strip is a standard DCap strip.

In some embodiments, the first side of the cartridge base includes a plurality of recesses, each configured to help align and/or secure a DCap in the plurality of DCap of the strip.

In some embodiments, the interface member is in a form of a plate and is substantially leveled with the cartridge base.

In some embodiments, the interface member includes: a first interface surface that is substantially planar; a second interface surface opposite to the first interface surface; a plurality of pin holes to facilitate alignment of a robotic EOAT with the interface member; an elongated slot formed through the first interface surface and configured in accordance with a cam bar of the robotic EOAT to allow insertion of the cam bar into the interface member; and a blind hole formed through the second interface surface and aligned with the elongated slot, wherein the blind hole has a bottom surface within the interface member to abut the cam bar of the robotic EOAT.

In an exemplary embodiment, the interface member is positioned such that the second surface is on the same side as the first side of the cartridge base.

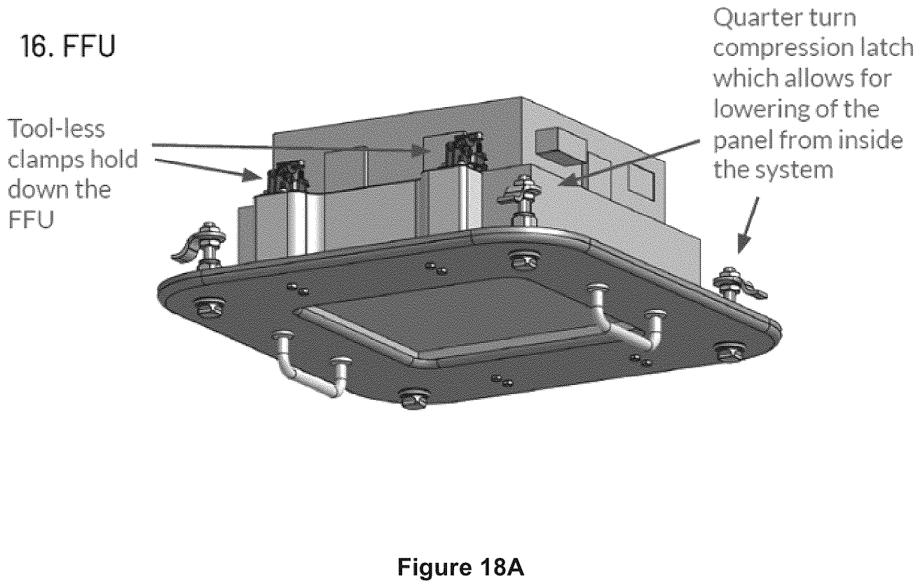

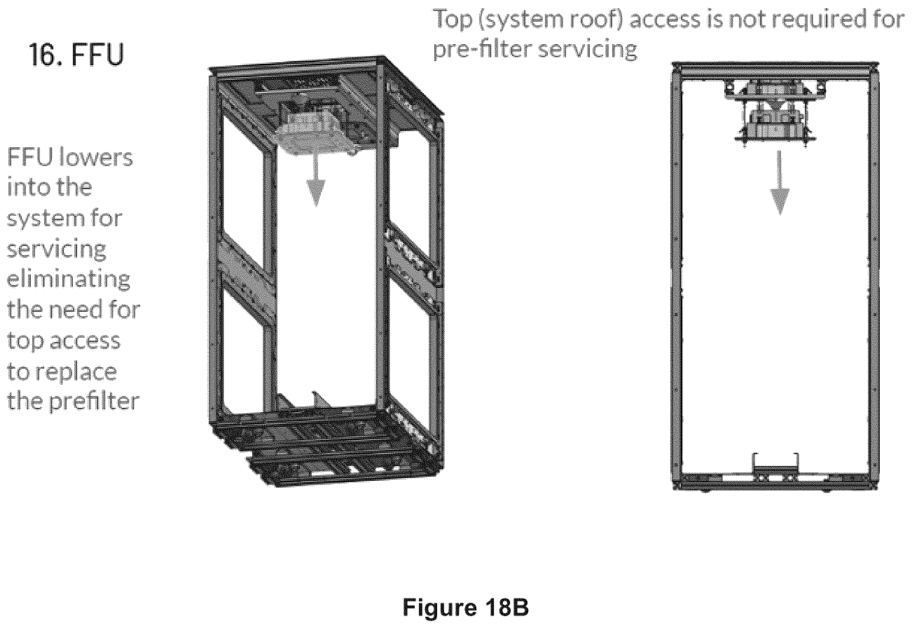

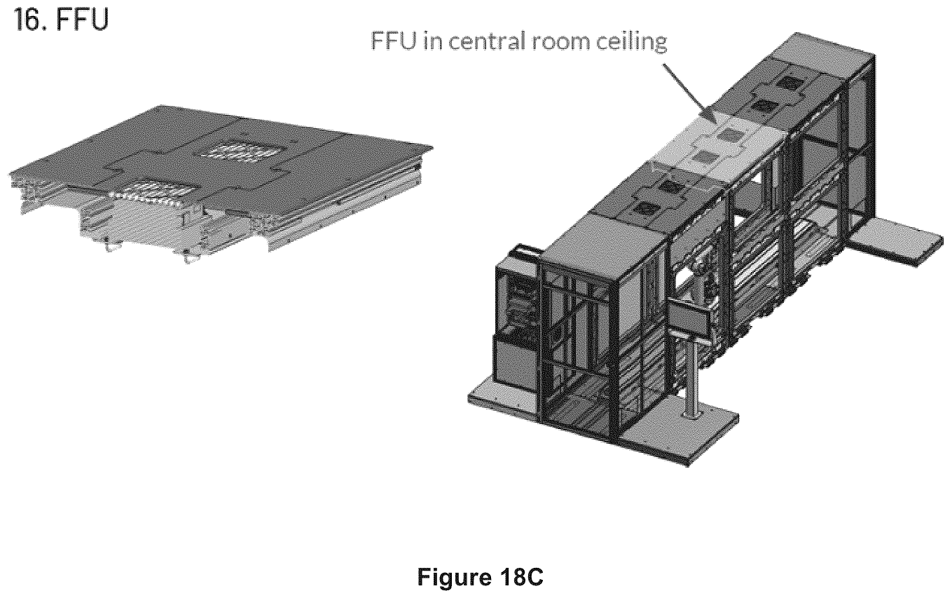

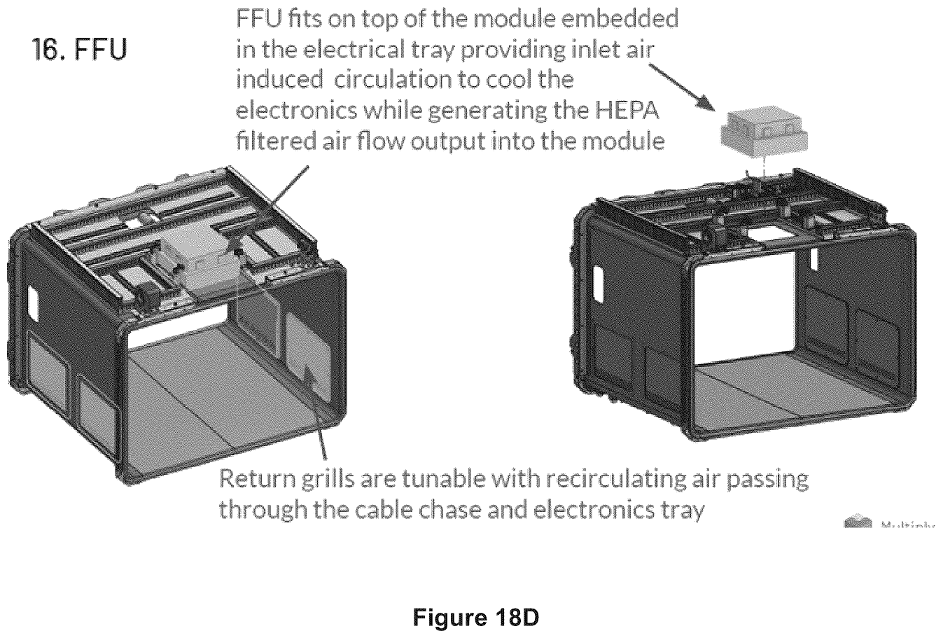

FIGS. 18A-18D illustrate an exemplary filtration system in accordance with some exemplary embodiments of the present disclosure. In various embodiments, the filtration system includes a novel design for mounting a fan filter unit (FFU) with a combination of tool-less fan and filter clamping and compression latch locking of the FFU access panel. One of the key innovations is the combination of a custom low profile Fan Filter Unit, tool-less installation, and interior-exterior service access, implicit plenum and ducts throughout the tower structures, and tunable return grills in an enclosed robotic system.

In some embodiments, the FFU includes a plurality of tool-less clamps for holding down the FFU, and a plurality of quarter turn compression latches for lowering a panel from inside a system.

In an exemplary embodiment, the FFU is installed at a lower side of an upper wall of the system, thereby eliminating a need for top access to replace a prefilter.

In another exemplary embodiment, the FFU is installed at a ceiling of a central room.

In still another exemplary embodiment, the FFU is installed on a top of a module embedded in an electrical tray to provide inlet air embedded induced circulation to cool electronics in the electrical tray and to generate filtered air flow output into the module.

In some embodiments, one or more return grills are disposed at one or more side walls of the module and are tunable with recirculating air passing through a cable chase and electronics tray.

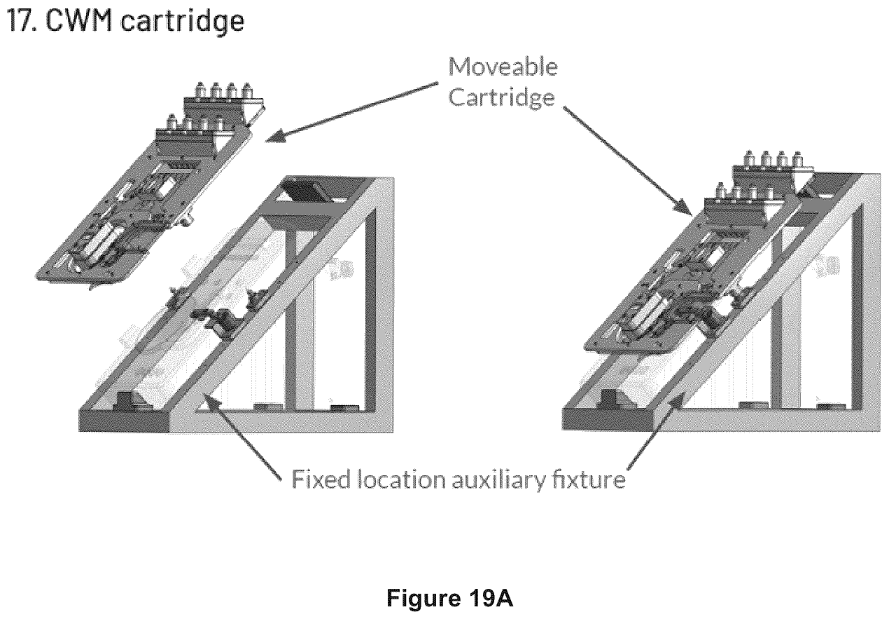

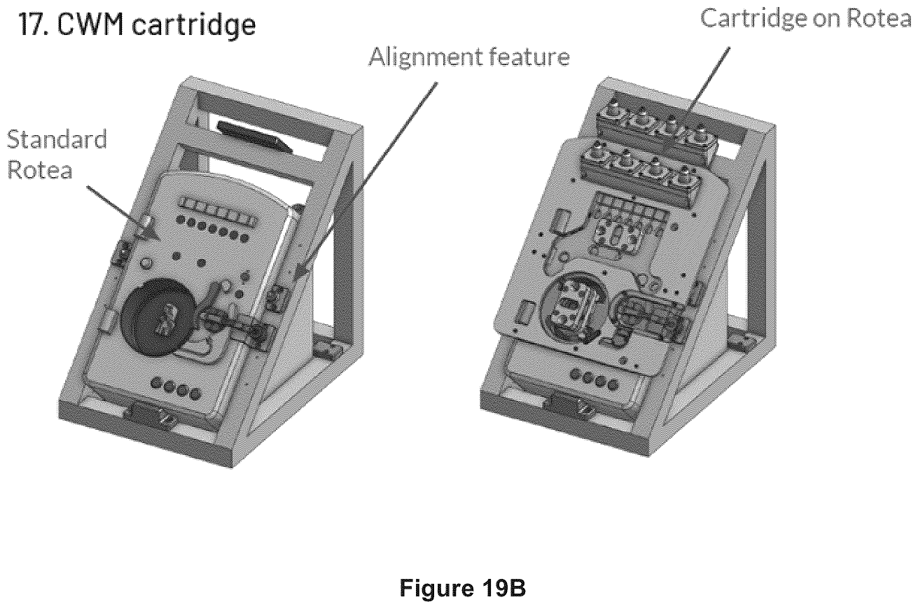

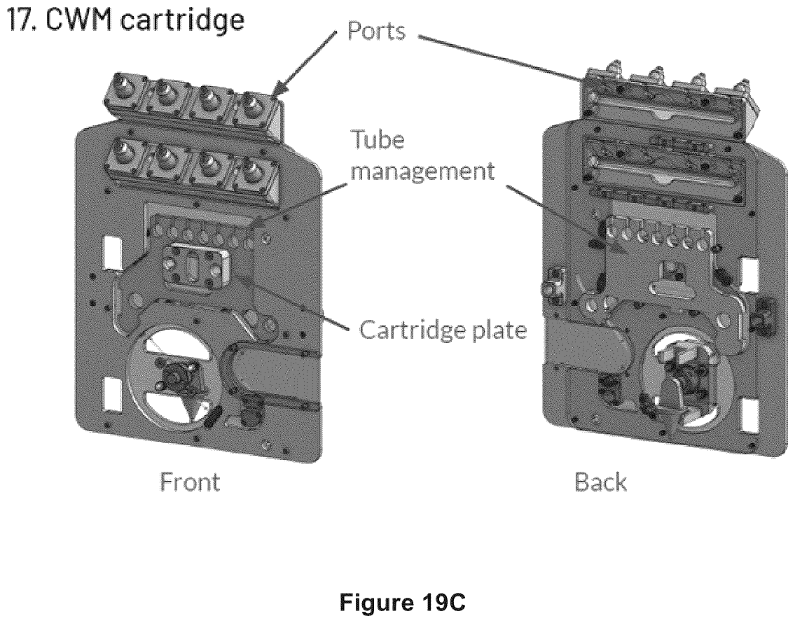

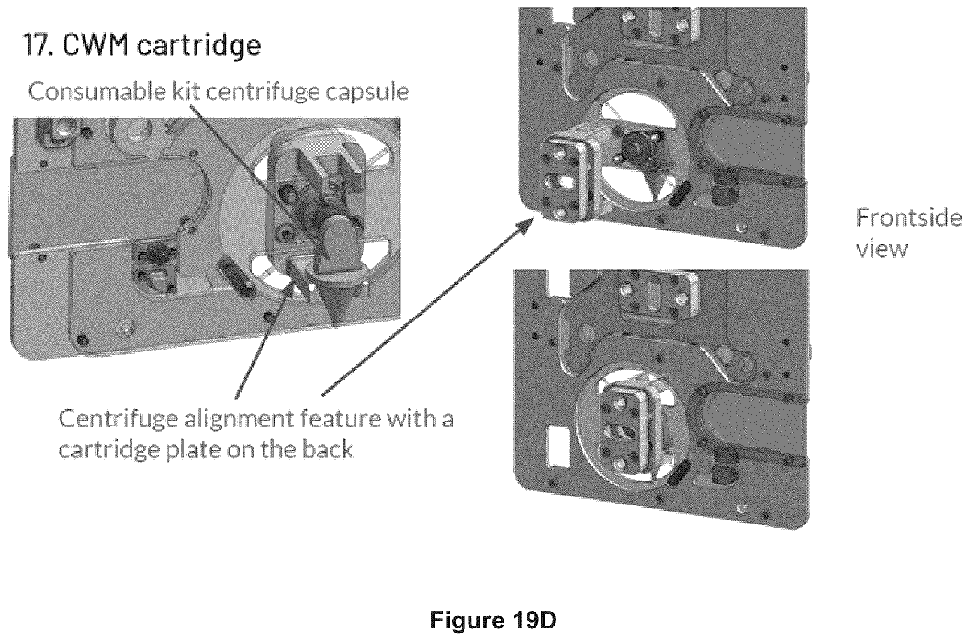

FIGS. 19A-19D illustrate a fifth exemplary cartridge in accordance with some exemplary embodiments of the present disclosure. In various embodiments, the cartridge includes a novel design that interfaces to the standard Rotea consumable kit and provides additional alignment and handling features to interface to a robot for automated loading and unloading into the Rotea. The key innovations include the use of a robot handled ‘preloaded’ cartridge with the consumable kit tubing aligned and retained in clips and features, custom rotating wiper and tubing insertion arm to stretch and seat the peristaltic tubing loop around the fixed location pump head, guide features to press tubing into the fixed location pinch valves, and robot handled device to capture and align the consumable kit centrifuge capsule.

In some embodiments, a device is configured for automated loading and unloading of a consumable kit to an instrument. The device includes a support for holding the instrument, and a cartridge. The cartridge includes: a mounting member for removably connecting the cartridge to the support at a first side of the instrument; a plurality of ports disposed at the mounting members; a tube management unit connected to or formed with the mounting member and configured to retain tubes that fluidly connect the consumable kit to the plurality of ports of the cartridge; and a first interface member connected to the mounting member and configured to be gripped by a robotic EOAT, thereby facilitating moving of the cartridge to or from the support.

In some embodiments, the instrument is a counterflow centrifugation system.

In some embodiments, the mounting member includes a first plate oblique to a ground and one or more platforms connected to or formed with the first plate. Each of the one or more platforms is substantially parallel to the ground. The plurality of ports is disposed at the one or more platforms in an upright position.

In some embodiments, a port in the plurality of ports is is a port disclosed herein (e.g., the port disclosed herein with respect to FIGS. 3A-3E).

In some embodiments, the tube management unit includes a second plate connected to the mounting member, wherein a plurality of holes and/or slots is formed at the second plate to retain the tubes of the consumable kit.

In some embodiments, the first interface member is disposed at the tube management unit and connected to the mounting member through the tube management unit.

In some embodiments, the device further includes a centrifuge alignment member removably connected to the mounting member and configured for selectively holding a centrifuge chamber or cone of the consumable kit.

In an exemplary embodiment, the centrifuge alignment member includes one or more jaw sets, each insertable through a hole formed on the mounting member and including a pair of jaws to selectively hold the centrifuge chamber or cone of the consumable kit.

In some embodiments, the device further includes a second inference member connected to the centrifuge alignment member and configured to be gripped by a robotic EOAT, thereby facilitating connecting and disconnecting the centrifuge alignment member with the mounting member.

In some embodiments, the device further includes a rotating wiper and tubing insertion arm to stretch and seat a peristaltic tubing loop around a fixed location pump head of the instrument.

In some embodiments, the device further includes one or more guide members to press tubing into one or more fixed location pinch valves of the instrument.

In some embodiments, the consumable kit is loaded to the cartridge before connecting the cartridge to the support.

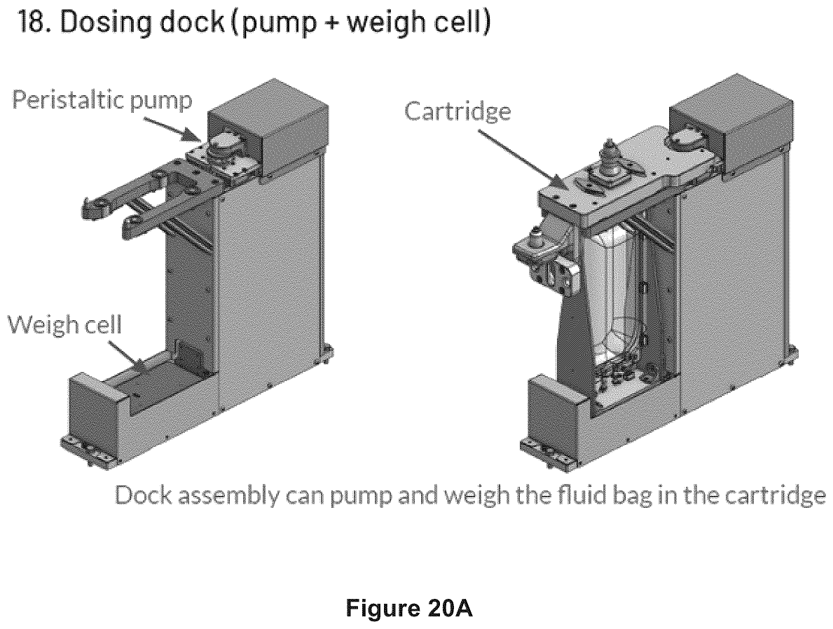

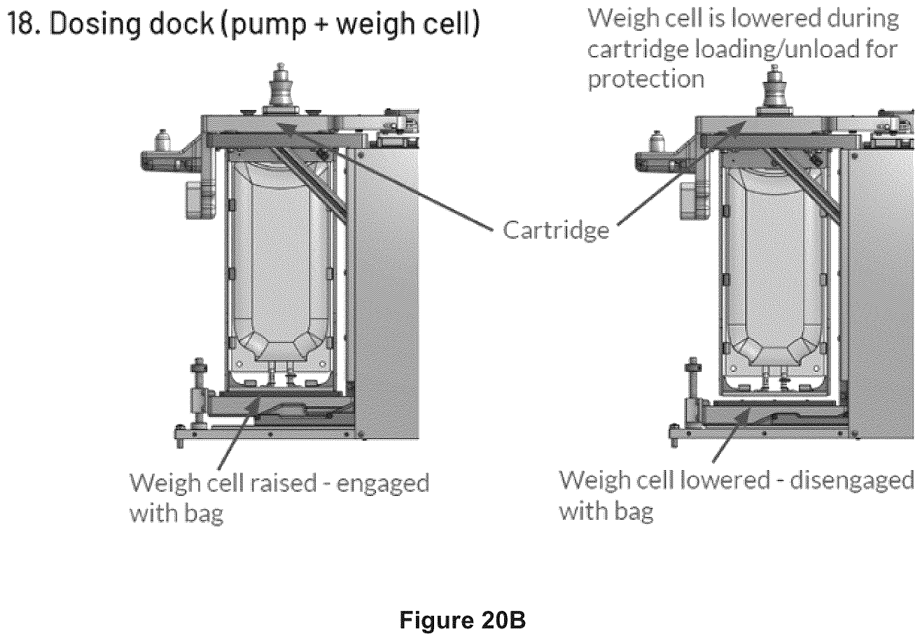

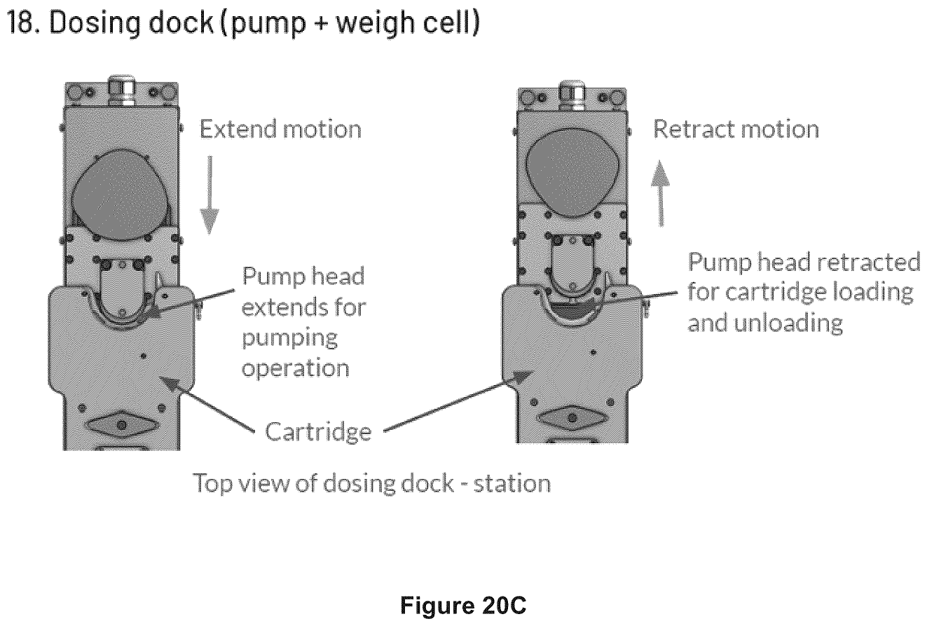

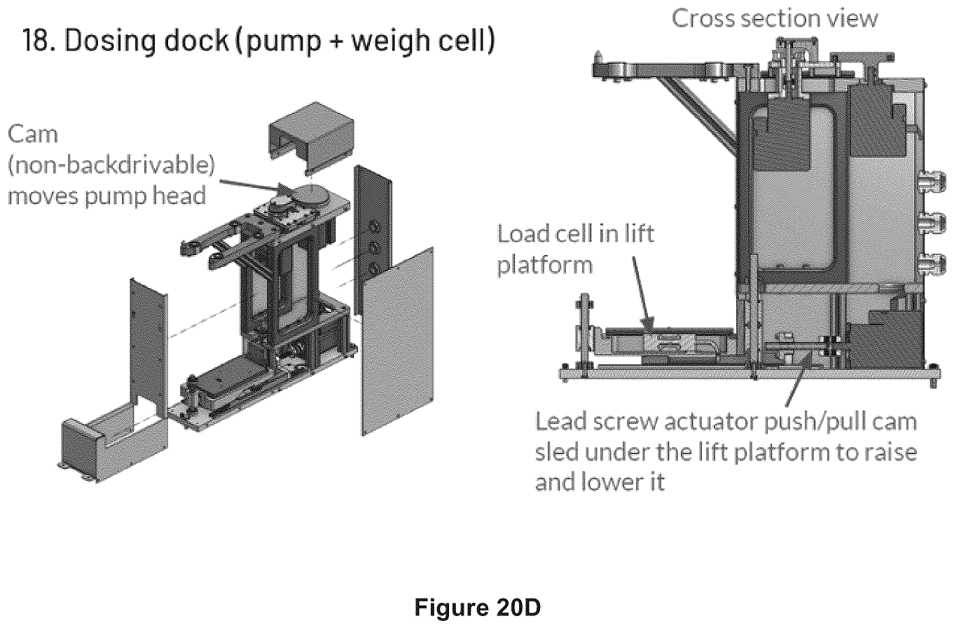

FIGS. 20A-20D illustrate a third exemplary dock in accordance with some exemplary embodiments of the present disclosure. In various embodiments, the dock includes a novel system incorporating both a sliding engagement peristaltic pump and cam driven lift stage for the weigh cell that enable a robot to automatically load and unload a dosing cartridge. One of the key innovations is the actuation of the pump head and weigh cell platform to move them to a neutral position to allow a dosing cartridge to be safely installed then the pump and weigh cell can be engaged after the cartridge is locked into position.

In some embodiments, a dosing dock includes a support, a pump unit and a weigh unit. The support is configured for mounting a dosing cartridge, wherein the dosing cartridge is configured to house a bag in fluid communication with a tubing disposed or formed at the dosing cartridge. The pump unit is configured for pumping a fluid to fill the bag through the tubing at the dosing cartridge, wherein the pump unit includes a pump head extendable and retractable to engage and disengage with the dosing cartridge. The weigh unit is configured for weighing a weight of the filled bag, wherein the weigh unit includes a lift platform movable in a vertical direction to engage and disengage with the bag.

In some embodiments, the dosing dock further includes a control unit to actuate the pump unit and the weigh unit.

In some embodiments, the pump is a peristaltic pump.

In some embodiments, the pump unit includes a cam mechanism to extend and retract the pump head.

In an exemplary embodiment, the pump head is moved substantially horizontally.

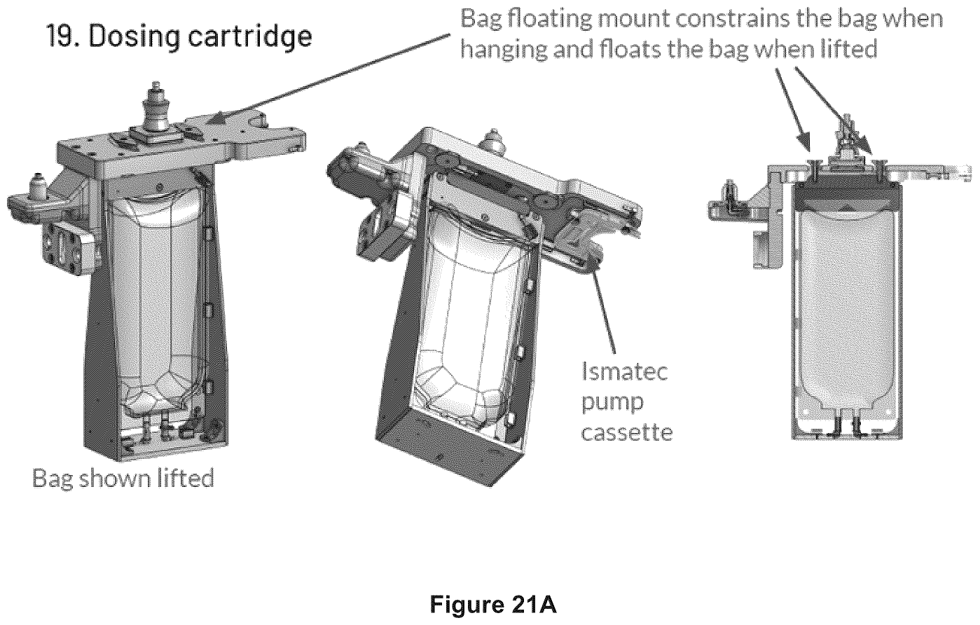

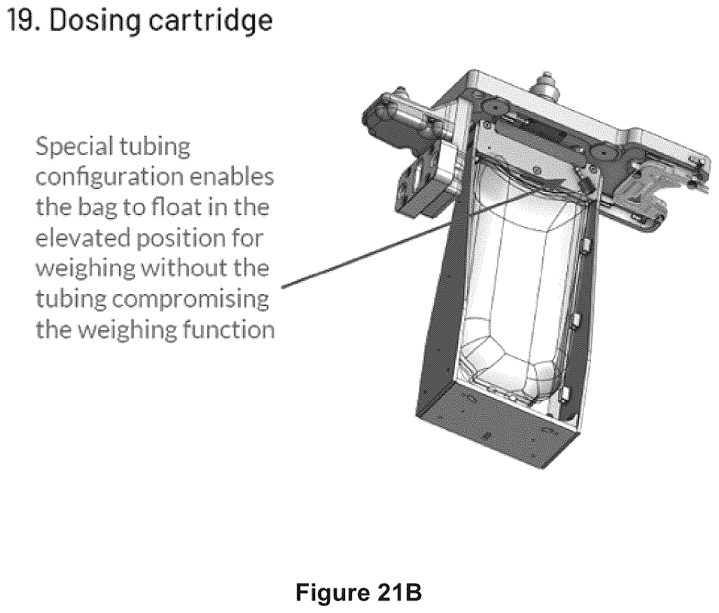

FIGS. 21A-21B illustrate a sixth exemplary cartridge in accordance with some exemplary embodiments of the present disclosure. In various embodiments, the cartridge includes custom cartridge design that houses a floating mount of a standard 1 liter fluid bag, tubing, and integral peristaltic pump cassette. One of the key innovations is the cartridge houses a combination of an Ismatec pump cassette, floating bag mount system with neutral loading integral tubing, dock locking features, and a robotic cartridge EOAT gripping feature.

In some embodiments, a dosing cartridge includes a mounting member, a compartment, a plurality of mounts, a pump cassette, a tubing and an interface member. The mounting member is configured for mounting the dosing cartridge on a dosing dock. The compartment is configured for housing a bag and connected to the mounting member. The plurality of mounts is disposed at the mounting member, wherein each mount in the plurality of mounts is operable between a hanging position and a lifted position with respect to the mounting member, thereby selectively containing the bag or floating the bag. The pump cassette is configured for coupling with a pump head of the dosing dock, wherein the pump cassette is disposed at the mounting member. The tubing is for fluidly connecting the bag to the pump cassette. The interface member is connected to the mounting member and configured to be gripped by a robotic EOAT, thereby facilitating moving of the dosing cartridge and docking of the dosing cartridge on the dosing dock.

In some embodiments, the dosing dock is a dosing dock disclosed herein (e.g., the dosing dock disclosed herein with respect to FIGS. 20A-20D).

In some embodiments, the dosing cartridge further includes one or more ports disposed at the mounting member.

In some embodiments, a port in the one or more is a port disclosed herein (e.g., the port disclosed herein with respect to FIGS. 3A-3E).

In some embodiments, the tubing is configured to enable the bag to float in an elevated position for weighing without the tubing compromising a weighing function.

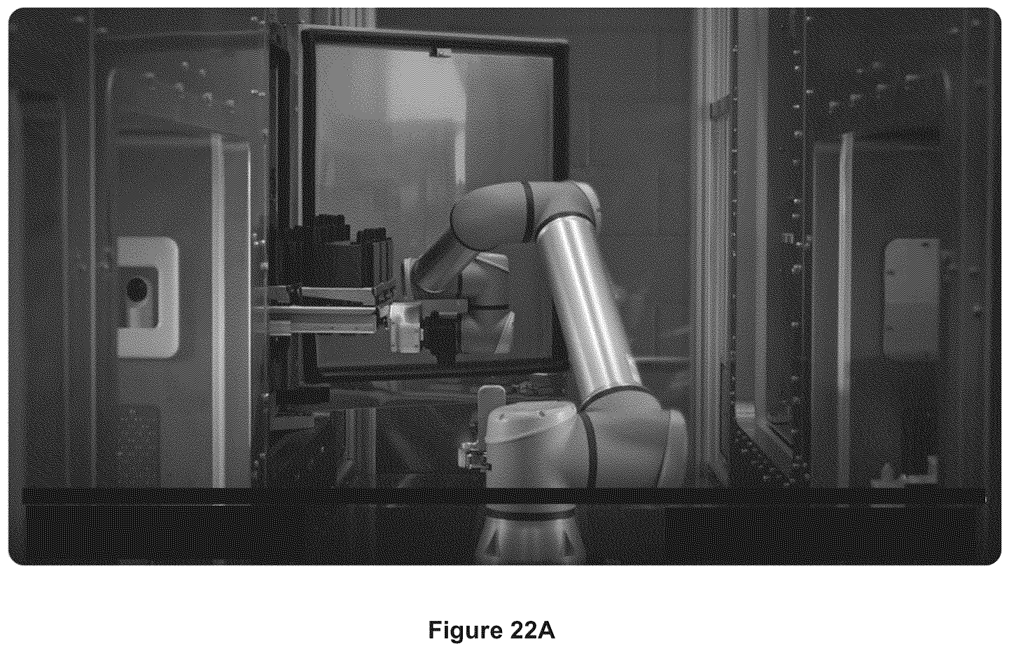

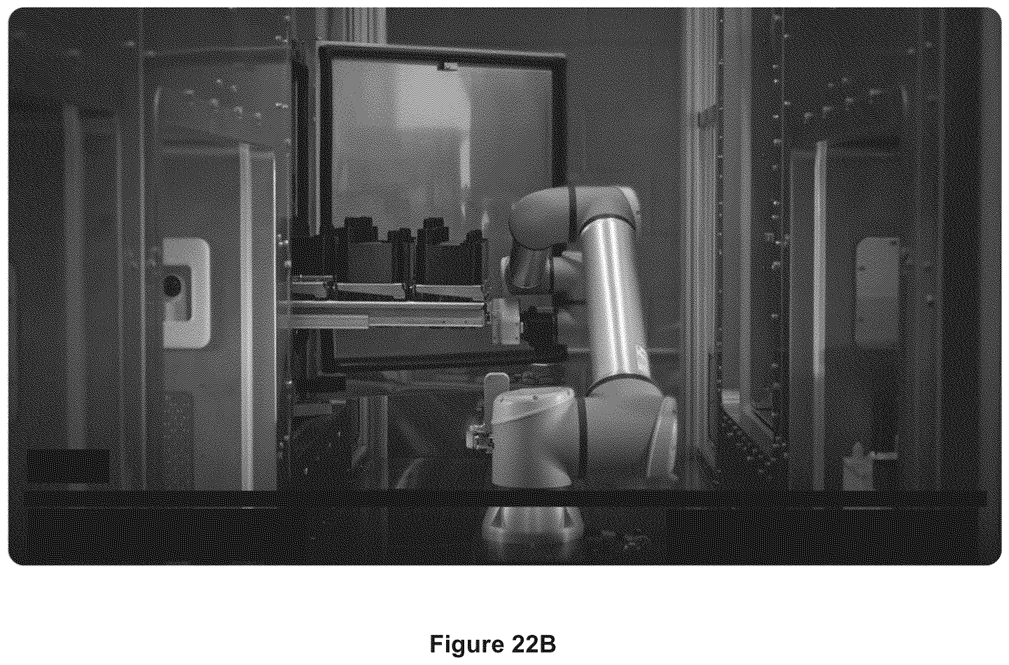

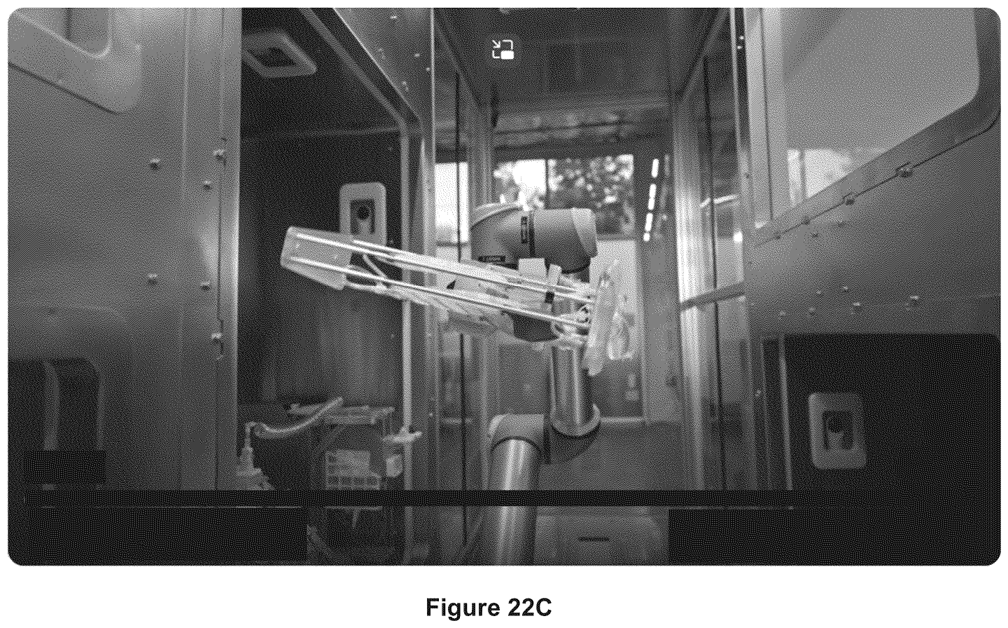

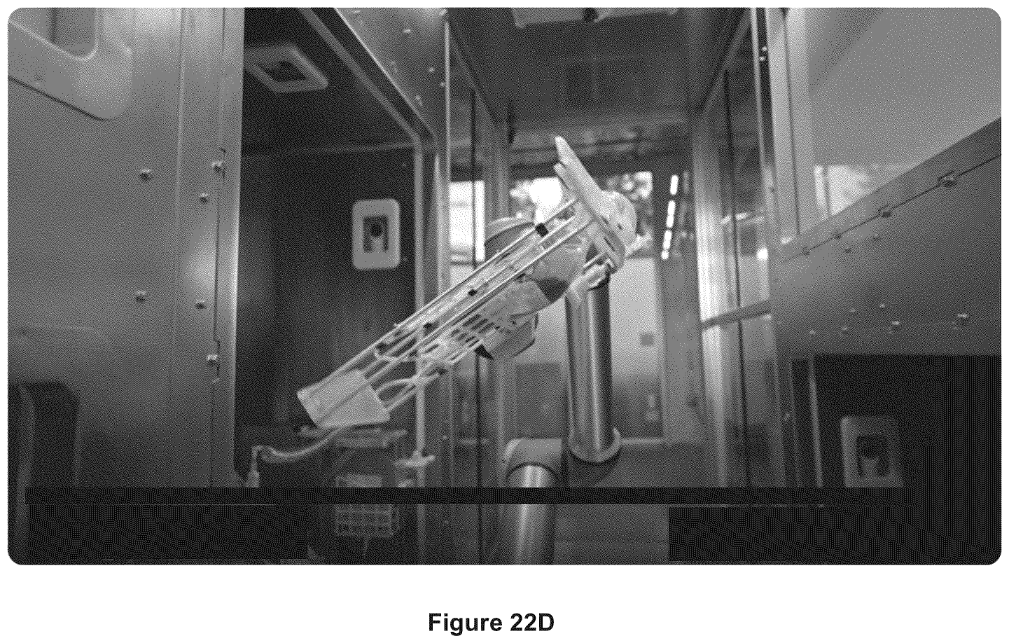

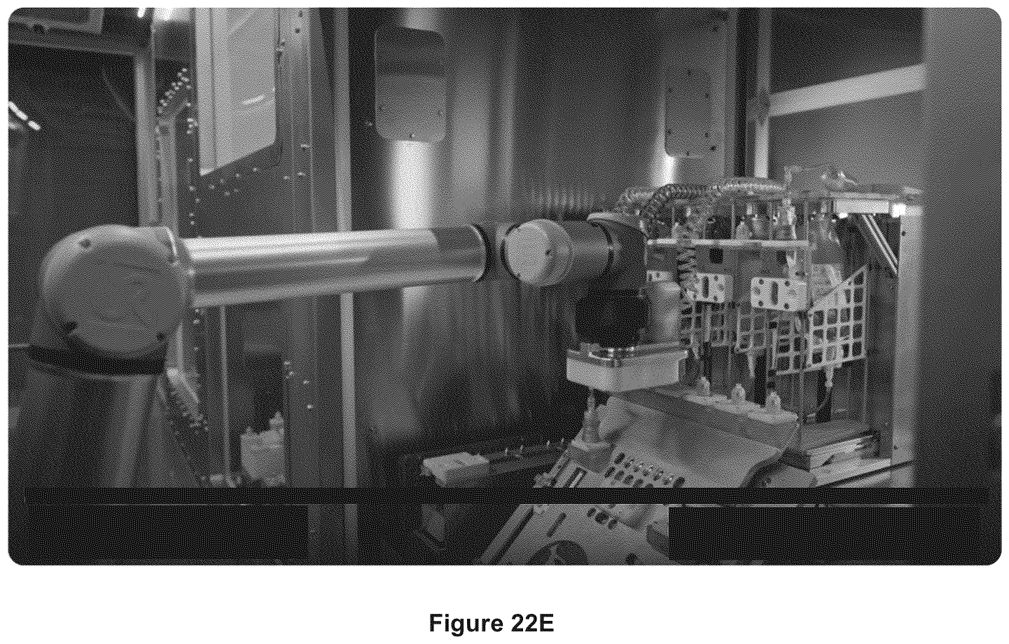

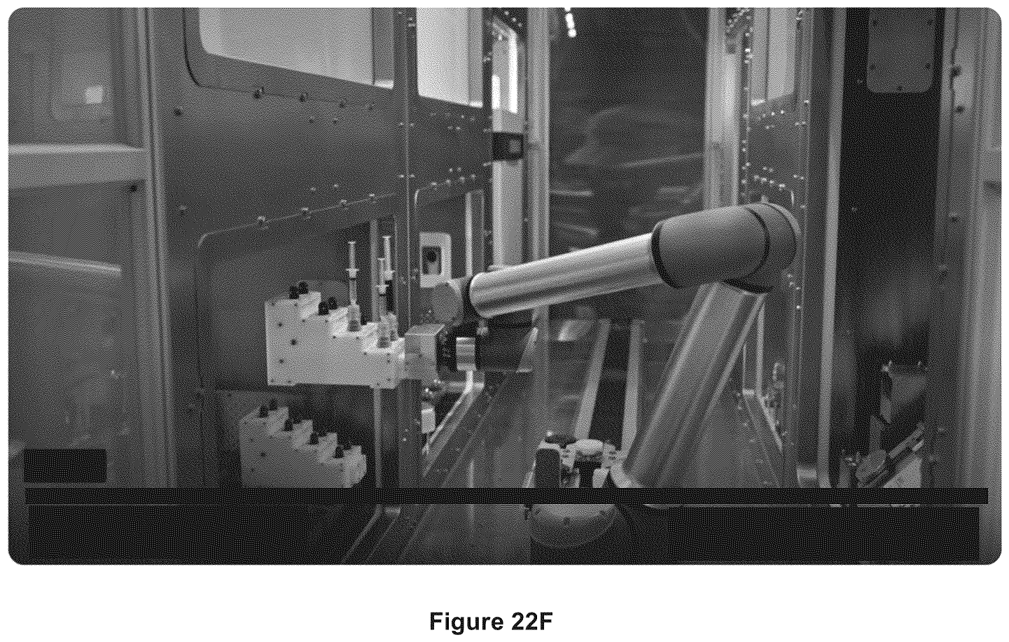

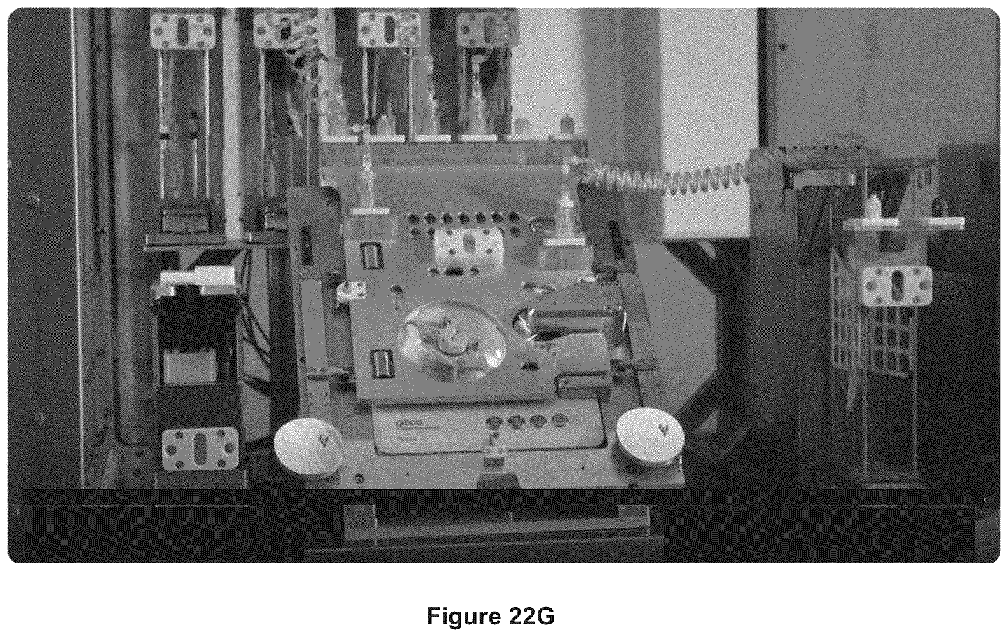

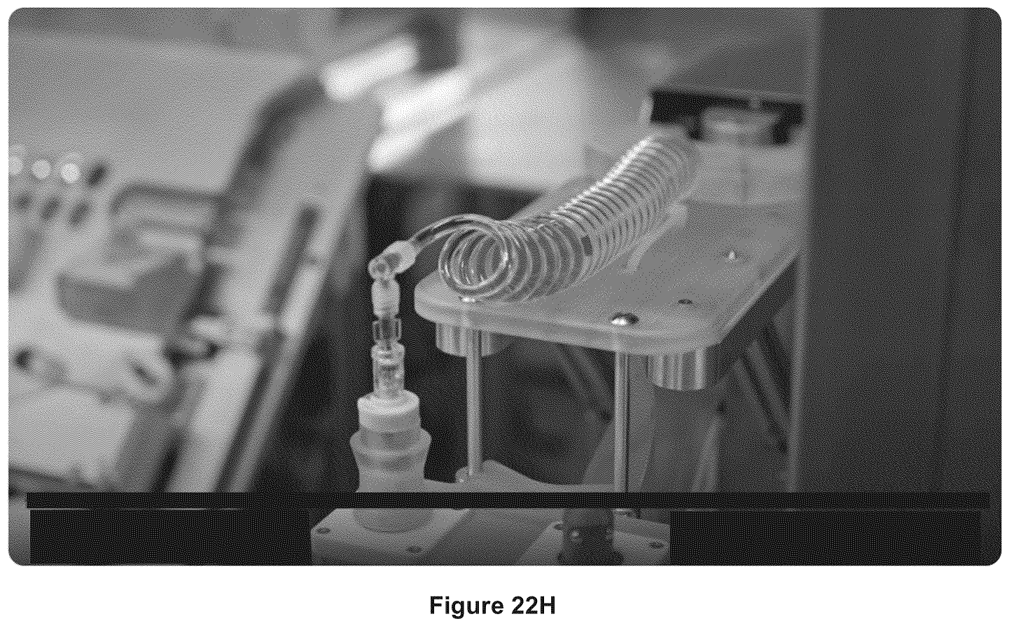

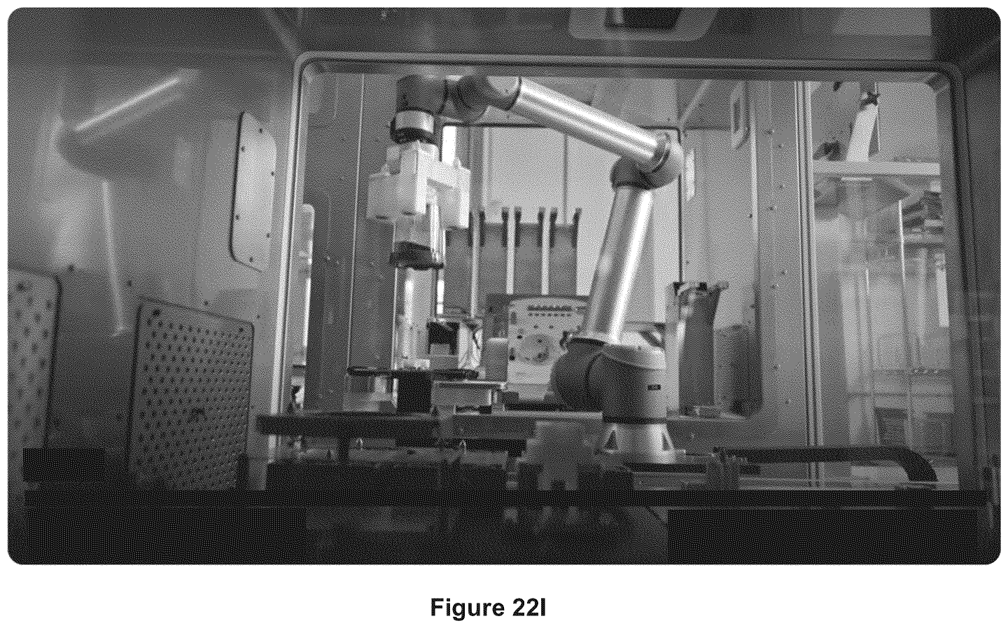

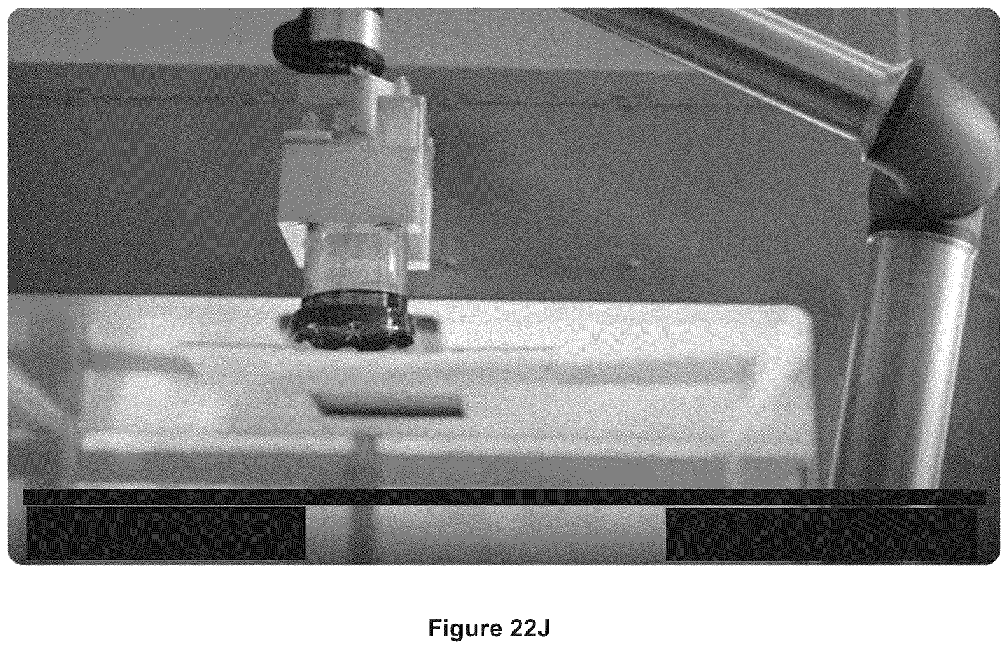

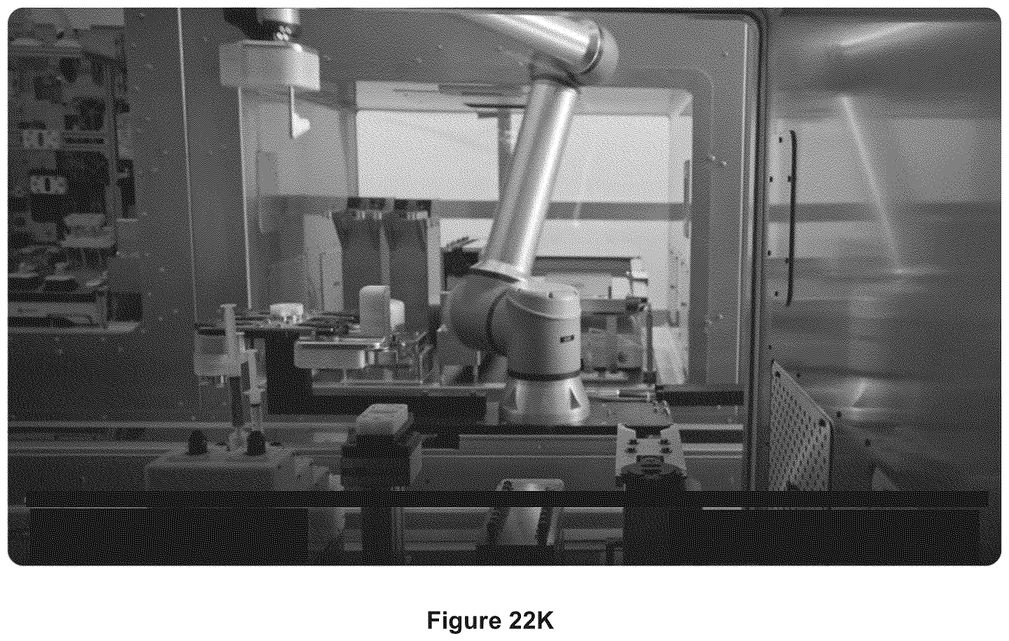

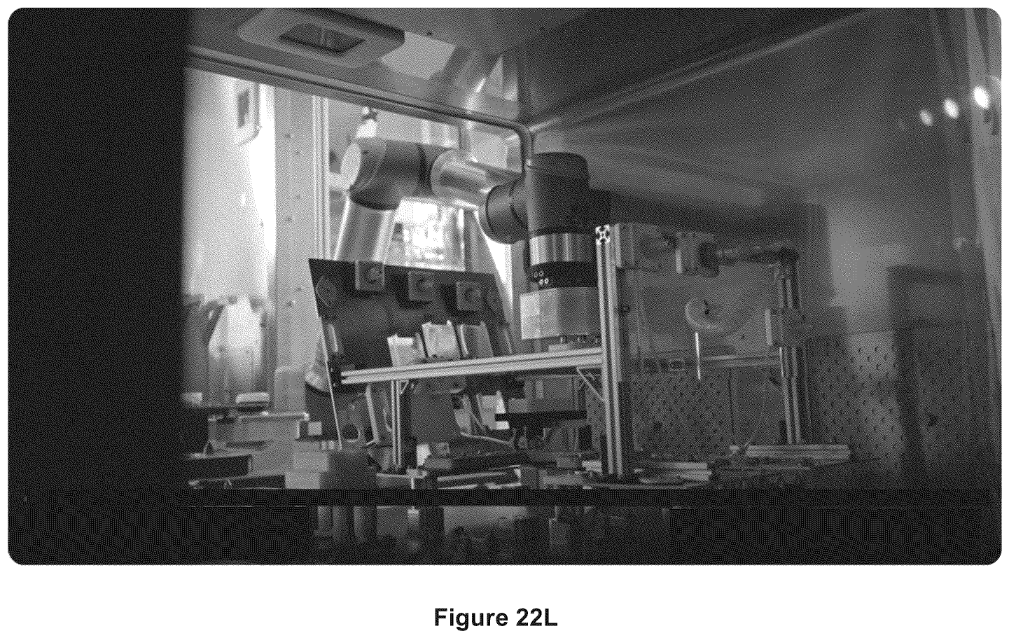

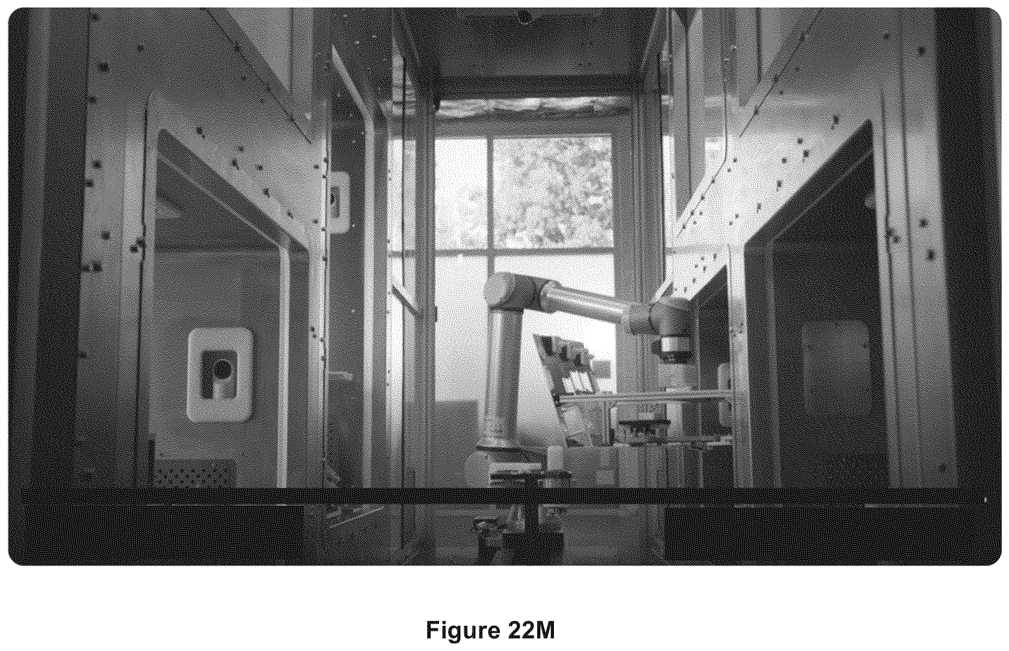

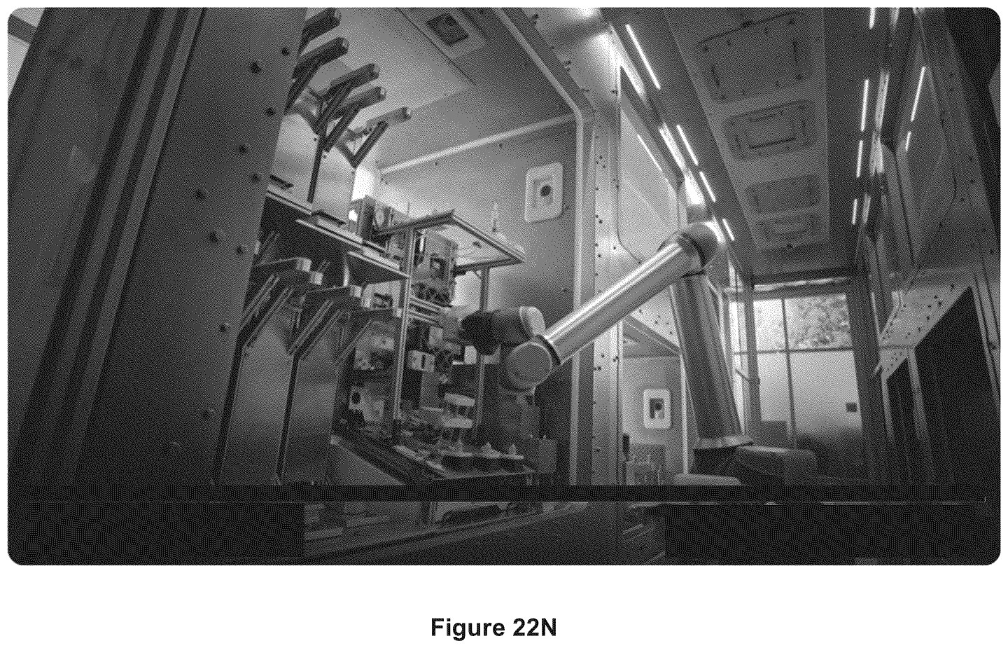

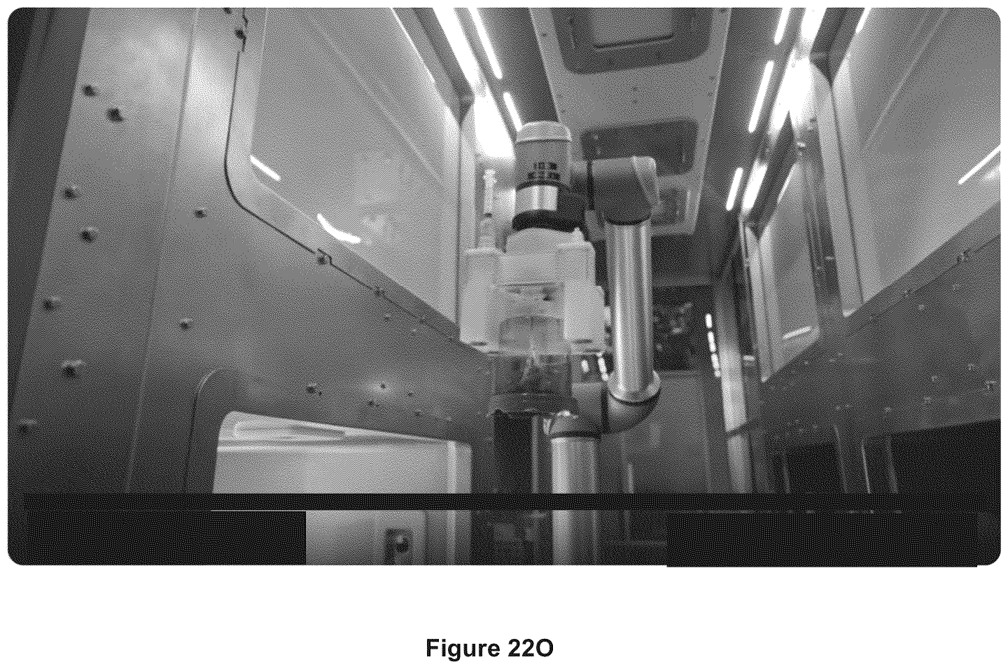

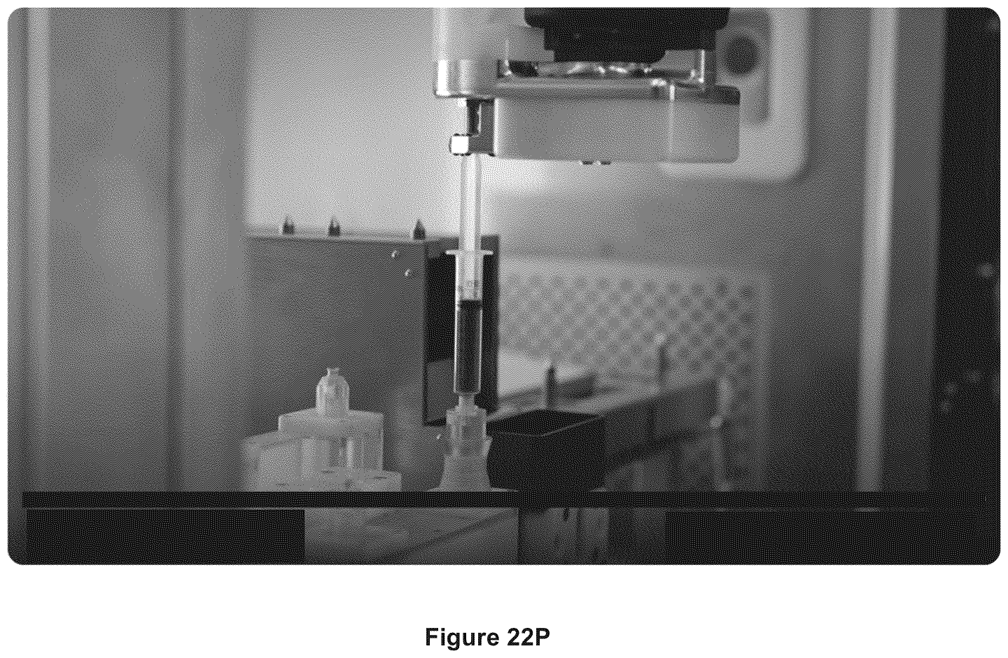

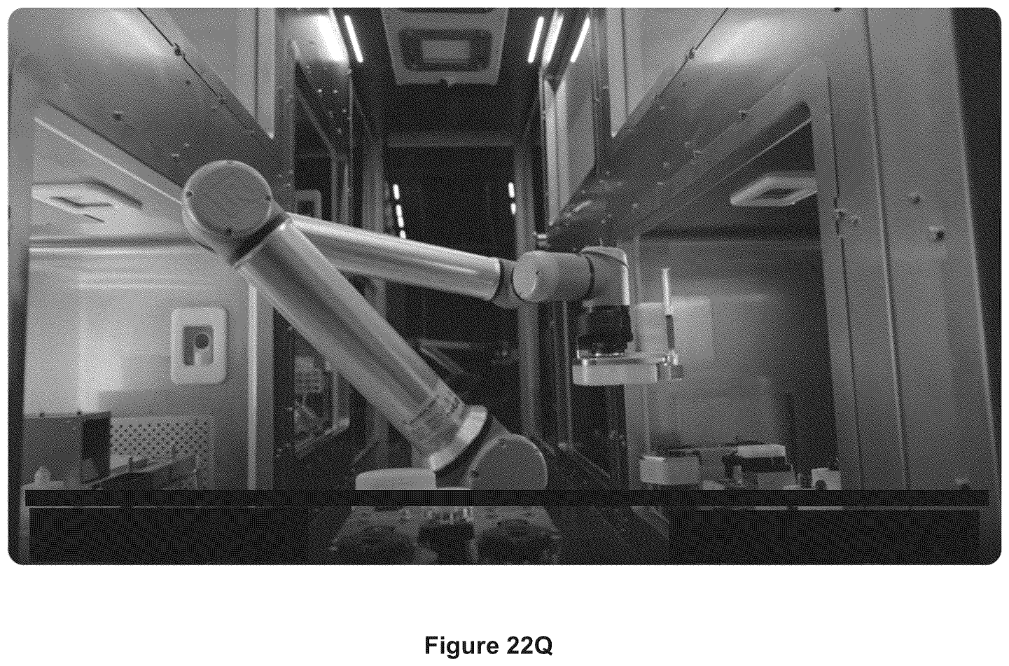

FIGS. 22A-22Q illustrate an exemplary biological foundry and exemplary operations of the biological foundry in accordance with some exemplary embodiments of the present disclosure.

In various embodiments, the present disclosure provides an apparatus for facilitating automated loading of a consumable kit to an instrument. The apparatus includes a cartridge and a dock. The cartridge includes a mounting member, a plurality of first docking members, and a plurality of first locking members. The mounting member is configured for holding the consumable kit at a rear side of the mounting member. The plurality of first docking members is coupled to or formed with the mounting member, and the plurality of first locking members is coupled to or formed with the mounting member. The dock is configured for securing the instrument. The dock includes a frame, a plurality of second docking members, and a plurality of second locking members. The frame is configured for surrounding at least a portion of a perimeter of the instrument at or adjacent to a front surface of the instrument. The plurality of second docking members is coupled to or formed with the frame. In some such embodiments, each respective second docking member in the plurality of second docking members is configured to removably couple with a corresponding first docking member in the plurality of first docking members. Coupling of each respective second docking member in the plurality of second docking members with the corresponding first docking member in the plurality of first docking members (a) restricts the mounting member of the cartridge from moving relative to the frame in a plane parallel or substantially parallel to the front surface of the instrument, and (b) allows the mounting member of the cartridge to move relative to the frame in a first direction perpendicular or substantially perpendicular to the front surface of the instrument. The plurality of second locking members is coupled to the frame. In some such embodiments, each respective second locking member in the plurality of second locking members is operably movable relative to the frame between a corresponding first position and a corresponding second position to selectively engage with or disengage from a corresponding first locking member in the plurality of first locking members. Engagement of each respective second locking member in the plurality of second locking members with the corresponding first locking member in the plurality of first locking members pushes the mounting member of the cartridge toward the front surface of the instrument and locks the mounting member of the cartridge with the frame of the dock.

In some embodiments, the instrument is a counterflow centrifugation system or the consumable kit is a closed system kit.

In some embodiments, the cartridge further includes a plurality of tube retaining sets. In some such embodiments, each respective tube retaining set in the plurality of tube retaining sets includes one or more corresponding tube retaining members disposed at the rear side of the mounting member and configured to retain a corresponding tube in a plurality of input or output tubes of the consumable kit.

In some embodiments, the one or more corresponding tube retaining members of each respective tube retaining set are disposed respectively at one or more corresponding locations on the rear side of the mounting member such that engagement of each respective second locking member in the plurality of second locking members with the corresponding first locking member in the plurality of first locking members pushes the corresponding tube in the plurality of input and output tubes of the consumable kit into a corresponding tubing track in a plurality of tubing tracks formed at the front surface of the instrument.

In an exemplary embodiment, each of the one or more corresponding tube retaining members of a respective tube retaining set is a mechanical fastener.

In some embodiments, the mounting member of the cartridge includes a plurality of holes or windows for visualizing flow in the plurality of input or output tubes of the consumable kit.

In some embodiments, the cartridge further includes a first interface member connected to or formed with the mounting member. The first interface member is configured for facilitating moving of the mounting member to or from the dock.

In an exemplary embodiment, the first interface member includes a first interface surface, a second interface surface, an elongated slot, and a recess. The first interface surface is accessible from a front side of the mounting member and is substantially planar. The second interface surface is opposite to the first interface surface. The elongated slot is formed through the first interface surface to allow an elongated cam bar of a robotic end of arm tool (EOAT) to insert into the first interface member. The recess is recessed from the second interface surface toward the first interface surface, wherein the recess has a dimension larger than a width of the elongated slot and a length of the elongated cam bar, thereby allowing the elongated cam bar of the EOAT to rotate and engage with the first interface member.

In some embodiments, the cartridge further includes a plurality of port units connected to the mounting member. In some such embodiments, each respective port unit in at least a subset of the plurality of port units is fluidly connected to a corresponding tube in a plurality of input and output tubes of the consumable kit.

In an exemplary embodiment, each of the plurality of first docking members includes a bushing, and each of the plurality of second docking members includes a post to removably couple with the bushing.

In some embodiments, each of the plurality of first locking members includes a ramp having a sloping surface with respect to the front surface of the instrument, and each of the plurality of second locking members includes a ramp follower operably movable on the sloping surface.

In some embodiments, the dock further includes a set of rails and a set of slides. Each rail in the set of rails is fixed on or formed with the frame. Each respective slide in the set of slides is coupled to a corresponding rail in the set of rails and operably movable along the corresponding rail. In some such embodiments, one or more second locking members in the plurality of second locking members are connected to or formed with each of the set of slides.

In some embodiments, the dock further includes a set of cam assemblies connected to the frame. In some such embodiments, an end portion of each respective slide in the set of slides is coupled to a corresponding cam assembly in the set of cam assemblies and the corresponding cam assembly converts a rotary motion to a linear motion of the respective slide.

In an exemplary embodiment, the plurality of first locking members includes four first locking members with two first locking members on each of a left side and a right side of the mounting member. The set of rails includes a left rail on a left side of the frame and a right rail on a right side of the frame. The set of slides includes a left slide coupled to the left rail and a right slide coupled to the right rail. The plurality of second locking members includes four second locking members with two second locking members on each of the left rail and the right rail.

In some embodiments, the dock further includes a plurality of face reference members disposed at a front surface of the frame. In some such embodiments, each of the plurality of face reference members includes a suspension beyond an inner edge of the frame to abut the front surface of the instrument, thereby aligning the front surface of the frame with the front surface of the instrument.

In an exemplary embodiment, each of the plurality of face reference members includes a pin fastened to the frame, wherein the pin is elongated in a direction parallel or substantially parallel to the front surface of the frame.

In some embodiments, the dock further includes a base for holding the instrument; and a plurality of upright members fixed on the base to support the frame such that the frame is disposed at a first angle with respect to the base.

In an exemplary embodiment, the dock further includes a plurality of stoppers fixed on the base, each adjustable and configured for abutting a wall of the instrument.

In some embodiments, the dock further includes a pump tube loading assembly connected to the frame and configured for placing at least a portion of a pump tube of the consumable kit into a peristaltic pump head of the instrument. The pump tube loading assembly includes a loading member and a driving unit. The loading member includes (i) a platform having a sector-shape or a substantial sector-shape and (ii) a finger disposed at or adjacent to a circumferential edge of the platform and extended toward the instrument beyond the platform. The driving unit is configured to rotate the loading member, thereby causing the finger to press at least the portion of the pump tube of the consumable kit into a peristaltic pump head to get it seated on rollers of the peristaltic pump head of the instrument and using the platform to prevent at least the portion of the pump tube of the consumable kit from popping out of the peristaltic pump head.

In an exemplary embodiment, the driving unit rotates the loading member around a rotational axis of the platform that is aligned with an axis of the peristaltic pump head of the instrument.

In some embodiments, the driving unit includes a plurality of shafts coupled to each other by a timing belt.

In some embodiments, the apparatus further includes a capsule releasing member and a second interface member. The capsule releasing member is configured for unlocking a centrifuge capsule of the consumable kit from a centrifuge chamber carrier of the instrument. The second interface member is connected to or formed with the capsule releasing member. The second interface member is robot-operable, thereby facilitating moving of the capsule releasing member relative to the instrument.

In an exemplary embodiment, the capsule releasing member includes a first jaw and a second jaw. The first jaw is insertable through a first hole formed on the mounting member and configured to grip the centrifuge capsule of the consumable kit. The second jaw is insertable through a second hole formed on the mounting member and configured to lift a lever in the centrifuge chamber carrier of the instrument, thereby unlocking the centrifuge capsule of the consumable kit from a centrifuge chamber carrier of the instrument.

In various embodiments, the present disclosure provides an apparatus for facilitating automated loading of a consumable kit to an instrument. The apparatus includes a cartridge and a dock. The cartridge is configured for holding the consumable kit and is movable by a robot. The dock is configured for securing the instrument. The dock includes a base, a frame, a plurality of upright members, and a plurality of face reference members. The base is configured for holding the instrument. The frame is configured for surrounding at least a portion of a perimeter of the instrument at or adjacent to a front surface of the instrument. The plurality of upright members is fixed on the base to support the frame such that the frame is disposed at a first angle with respect to the base. The plurality of face reference members is disposed at a front surface of the frame. In some such embodiments, each of the plurality of face reference members includes a suspension beyond an inner edge of the frame to abut the front surface of the instrument, thereby aligning the front surface of the frame with the front surface of the instrument.

In some embodiments, each of the plurality of face reference members includes a pin that is parallel or substantially parallel to the front surface of the frame and fastened to the frame.

In an exemplary embodiment, the dock further includes a plurality of stoppers fixed on the base, each adjustable and configured for abutting a wall of the instrument.

In various embodiments, the present disclosure provides an apparatus for facilitating automated loading of a consumable kit to an instrument. The apparatus includes a cartridge and a dock. The cartridge is configured for holding the consumable kit and is movable by a robot. The dock is configured for securing the instrument. The dock includes a frame and a pump tube loading assembly. The frame is configured for surrounding at least a portion of a perimeter of the instrument at or adjacent to a front surface of the instrument. The pump tube loading assembly is connected to the frame and configured for placing at least a portion of a pump tube of the consumable kit into a peristaltic pump head of the instrument. The pump tube loading assembly includes a loading member and a driving unit. The loading member includes (i) a platform having a sector-shape or a substantial sector-shape and including a retaining surface that faces the instrument, wherein the retaining surface is parallel or substantially parallel to the front surface of the instrument, and (ii) a finger disposed at or adjacent to a circumferential edge of the platform and extended toward the instrument beyond the retaining surface of the platform. The driving unit is configured to rotate the loading member, thereby causing the finger to press at least the portion of the pump tube of the consumable kit into a peristaltic pump head to seat on rollers of the peristaltic pump head of the instrument and causing the platform to confine at least the portion of the pump tube of the consumable kit to prevent the pump tube of the consumable kit from popping out of the peristaltic pump head.

In some embodiments, the driving unit rotates the loading member around a rotational axis of the platform that is aligned with an axis of the peristaltic pump head of the instrument.

In various embodiments, the present disclosure provides an apparatus for facilitating automated loading of a consumable kit to an instrument. The apparatus includes a cartridge and a dock. The cartridge is movable by a robot and includes a mounting member, a plurality of first locking members, and a plurality of tube retaining sets. The mounting member is configured for holding the consumable kit at a back side of the mounting member. The plurality of first locking members is coupled to or formed with the mounting member. Each respective tube retaining set in the plurality of tube retaining sets includes one or more corresponding tube retaining members disposed at the back side of the mounting member and configured to retain a corresponding tube in a plurality of input and output tubes of the consumable kit. The dock is configured for securing the instrument and includes a frame and a plurality of second locking members. The frame is configured for surrounding at least a portion of a perimeter of the instrument at or adjacent to a front surface of the instrument. The plurality of second locking members is coupled to the frame. In some such embodiments, each respective second locking member in the plurality of second locking members is configured to selectively engage with or disengage from a corresponding first locking member in the plurality of first locking members. The one or more corresponding tube retaining members of each respective tube retaining set are disposed respectively at one or more corresponding locations on the back side of the mounting member such that engagement of each respective second locking member in the plurality of second locking members with the corresponding first locking member in the plurality of first locking members pushes the corresponding tube in the plurality of input and output tubes of the consumable kit into a corresponding tubing track in a plurality of tubing tracks formed at the front surface of the instrument.

In some embodiments, the dock further includes a pump tube loading assembly connected to the frame and configured for placing at least a portion of a pump tube of the consumable kit around a peristaltic pump head of the instrument. The pump tube loading assembly includes a loading member and a driving unit. The loading member includes (i) a platform having a sector-shape or a substantial sector-shape and (ii) a finger disposed at or adjacent to a circumferential edge of the platform and extended toward the instrument beyond the platform. The driving unit is configured to rotate the loading member, thereby causing the finger to press at least the portion of the pump tube of the consumable kit into a peristaltic pump head to get it seated on rollers of the peristaltic pump head of the instrument and using the platform to prevent at least the portion of the pump tube of the consumable kit from popping out of the peristaltic pump head.

In various embodiments, the present disclosure provides a cartridge for facilitating automation of a vessel having a cap and at least one vessel port. The cartridge includes a cartridge body, an adapter and at least one connection unit. The cartridge body includes a side wall, an upper wall and an interior, where the upper wall is connected to or formed with an upper portion of the side wall, and the interior is defined by the side wall and the upper wall. The adapter is disposed in the interior of the cartridge body and connected to or formed with the cartridge body, and configured for removably connecting the vessel to the cartridge body. Each respective connection unit in the at least one connection unit includes a corresponding retainer, a corresponding port body, a corresponding connector and a corresponding tube. The corresponding retainer is fixed on or integrally formed with the upper wall of the cartridge body. The corresponding port body is coupled to the corresponding retainer and has a corresponding distal end portion positioned exterior to the cartridge body. The corresponding connector is housed by the corresponding port body and has a corresponding first end and a corresponding second end, where the corresponding first end is positioned outside of the cartridge body. The corresponding tube is configured for connecting the corresponding second end of the corresponding connector to a vessel port in the at least one vessel port of the vessel.

In some embodiments, the vessel is a gas permeable rapid expansion bioreactor device.

In some embodiments, the at least one vessel port is formed at the cap of the vessel.

In some embodiments, an interior surface of the side wall includes a first interior surface segment, a second interior surface segment, and a third interior surface segment between the first and second interior surface segments. In some such embodiments, the second interior surface segment is parallel or substantially parallel to the first interior surface segment. Each of the first and second interior surface segments is planar or substantially planar, and the third interior surface segment is curved in accordance with the vessel.

In some embodiments, an exterior surface of the side wall includes a first exterior surface segment, a second exterior surface segment, and a third exterior surface segment between the first and second exterior surface segments. In some such embodiments, the second exterior surface segment is parallel or substantially parallel to the first exterior surface segment. Each of the first, second, and third exterior surface segments is planar or substantially planar, and the third exterior surface segment is perpendicular or substantially perpendicular to the first and second exterior surface segments.

In some embodiments, the at least one connection unit includes a plurality of connection units.

In some embodiments, the upper wall of the cartridge body includes a first upper wall segment and a second upper wall segment, where the first and second upper wall segments are at different heights. In some such embodiments, at least one retainer in the corresponding retainers of the plurality of connection units is fixed on or integrally formed with each of the first and second upper wall segments.

In an exemplary embodiment, more than one retainer in the corresponding retainers of the plurality of connection units are fixed on or integrally formed with the first or second upper wall segment.

In some embodiments, the corresponding port body is movable translationally relative to the corresponding retainer in a plane perpendicular or substantially perpendicular to an axial direction of the corresponding port body.

In some embodiments, for each respective connection unit in the at least one connection unit, the corresponding port body includes a base, a stem extending from the base, and one or more first anti-rotation members disposed at the base. The corresponding retainer includes a first retaining member and a second retaining member coupled or formed with the first retaining member. The first retaining member has a first surface, and the second retaining member has a second surface spaced apart from the first surface of the first retaining member in an axial direction of the corresponding port body, where the base of the corresponding port body is disposed between the first surface of the first retaining member and the second surface of the second retaining member. The corresponding retainer also includes a first circular or substantially circular through-hole disposed on the first retaining member, The first circular or substantially circular through-hole has a diameter larger or substantially larger than an outer diameter of the stem to allow the stem of the corresponding port body to pass through and to move relative to the first retaining member. The corresponding retainer further includes one or more second anti-rotation members disposed at the first retaining member or the second retaining member and coupled with the one or more first anti-rotation members to restrict the corresponding port body from rotating relative to the retainer.

In some embodiments, the second retaining member of the corresponding retainer of each respective connection unit in the at least one connection unit is a portion of the upper wall of the cartridge body.

In an exemplary embodiment, the adapter is coupled to the upper wall of the cartridge body and the cap of the vessel.

In some embodiments, the adapter includes at least one slot for accommodating the at least on vessel port, the corresponding tube of each respective connection unit in at least one connection unit, or any combination thereof.

In some embodiments, the adapter includes (i) an internal flange for inserting into a gap between the cap and a body of the vessel, and (ii) a plurality of internal ribs for abutting a side wall of the cap, thereby restricting the cap of the vessel from moving relative to the cartridge body.

In some embodiments, the corresponding retainer restricts axial and rotational movement of the corresponding port body but allows translational movement of the corresponding port body relative to the corresponding retainer in a plane perpendicular or substantially perpendicular to an axial direction of the corresponding port body.

In some embodiments, the cartridge includes an interface member disposed at the side wall or the upper wall of the cartridge body to facilitate operation by a robotic end of arm tool (EOAT).

In an exemplary embodiment, the interface member is disposed at a middle portion of the upper wall of the cartridge body.

In some embodiments, the interface member includes a first interface surface, a second interface surface, an elongated slot, and a recess. The first interface surface is planar or substantially planar and faces away from the side wall or the upper wall of the cartridge body. The second interface surface faces toward the side wall or the upper wall of the cartridge body. The elongated slot is formed through the first interface surface to allow an elongated cam bar of the EOAT to insert into the interface member. The recess is recessed from the second interface surface toward the first interface surface and has a dimension larger than a width of the elongated slot and a length of the elongated cam bar, thereby allowing the elongated cam bar of the EOAT to rotate and engage with the interface member.

In an exemplary embodiment, the recess is a circular blind hole formed through the second interface surface and aligned with the elongated slot.

In some embodiments, the recess has a bottom surface within the interface member that is curved or slanted relative to the first interface surface.

In some embodiments, the cartridge includes a plurality of first alignment elements to facilitate alignment of the interface member with the EOAT.

In an exemplary embodiment, a first alignment element in the plurality of first alignment elements is a pin or a pin hole.

In some embodiments, the cartridge includes a plurality of second alignment elements formed at a lower portion of the side wall of the cartridge body to facilitate alignment and positioning of the cartridge on a dock.

In an exemplary embodiment, a second alignment element in the plurality of second alignment elements is a pin or a pin hole.

In some embodiments, the cartridge includes a plurality of locking elements disposed at a lower portion of the side wall of the cartridge body to facilitate positioning and locking of the cartridge on a dock

In an exemplary embodiment, a locking element in the plurality of locking elements is an electromagnet or an electromagnet target.

In various embodiments, the present disclosure provides a cartridge for facilitating automation of a vessel having a cap and at least one vessel port. The cartridge includes a cartridge body, an adapter, at least one connection unit, and an interface member. The cartridge body includes an interior space to receiving at least a portion of the vessel. The adapter is disposed in the interior space and connected to or formed with the cartridge body, wherein the adapter is configured for connecting the vessel to the cartridge body. Each respective connection unit in at least one connection unit includes a corresponding connector, where the corresponding connector has a corresponding first end accessible from outside of the cartridge body and a corresponding second end for coupling with a vessel port in the at least one vessel port. The interface member is disposed on the cartridge body to facilitate operation by a robotic end of arm tool (EOAT). The interface member includes a first interface surface, a second interface surface, an elongated slot, and a recess. The first interface surface is planar or substantially planar and faces away from the cartridge body. The second interface surface faces toward the cartridge body. The elongated slot is formed through the first interface surface to allow an elongated cam bar of the EOAT to insert into the interface member. The recess is recessed from the second interface surface toward the first interface surface and has a dimension larger than a width of the elongated slot and a length of the elongated cam bar, thereby allowing the elongated cam bar of the EOAT to rotate and engage with the interface member.

In various embodiments, the present disclosure provides a cartridge for facilitating automation of a vessel having a cap and a plurality of vessel ports. The cartridge includes a cartridge body, an adapter, and a plurality of connection units. The cartridge body includes an upper wall having a plurality of regions. The adapter is connected to or formed with the cartridge body, and configured for removably connecting the vessel to the cartridge body. Each respective connection unit in the plurality of connection unit includes a corresponding port body, a corresponding retainer, a corresponding connector, and a corresponding tube. The corresponding port body includes a base, a stem extending from the base, and one or more first anti-rotation members disposed at the base. The corresponding retainer includes a first retaining member, a second retaining member, a first circular or substantially circular through-hole, and one or more second anti-rotation members. The second retaining member is a corresponding region in the plurality of regions of the upper wall of the cartridge body. The first retaining member has a first surface and the second retaining member has a second surface spaced apart from the first surface of the first retaining member in an axial direction of the corresponding port body, with the base of the corresponding port body disposed between the first surface of the first retaining member and the second surface of the second retaining member. The first circular or substantially circular through-hole is disposed on the first retaining member and has a diameter larger or substantially larger than an outer diameter of the stem to allow the stem of the corresponding port body to pass through and to move relative to the first retaining member. The one or more second anti-rotation members are disposed at the first retaining member or the second retaining member and coupled with the one or more first anti-rotation members to restrict the corresponding port body from rotating relative to the retainer. The corresponding connector is housed by the corresponding port body and has a corresponding first end and a corresponding second end, where the corresponding first end is positioned outside of the cartridge body. The corresponding tube is configured for connecting the corresponding second end of the corresponding connector to a vessel port in the plurality of vessel ports.

In some embodiments, the upper wall of the cartridge body includes a first upper wall segment and a second upper wall segment, where the first and second upper wall segments are at different heights. In some such embodiments, each of the first and second upper wall segments includes at least one region in the plurality of regions to serve as the second retaining member of at least one retainer.

In some embodiments, each of the first and second upper wall segments includes two or more regions in the plurality of regions to serve as the second retaining members of two or more retainers.

In various embodiments, the present disclosure provides an apparatus for facilitating automated connection of a first device and a second device. The apparatus includes a port body and a retainer. The port body includes a base, a stem, a bore, a tip and one or more first anti-rotation members. The stem extends from the base. The bore extends from an upper end portion of the stem to a lower end portion of the base and configured for receiving at least a portion of the first device. The tip is disposed at a free end portion of the stem and configured for guiding the second device when connecting the second device and the first device. The one or more first anti-rotation members are disposed at the base. The retainer includes a first retaining member, a second retaining member, a first circular or substantially circular through-hole, and one or more second anti-rotation members. The first retaining member has a first surface. The second retaining member is coupled or formed with the first retaining member and has a second surface spaced apart from the first surface of the first retaining member in an axial direction of the port body. In some such embodiments, the base of the port body is disposed between the first surface of the first retaining member and the second surface of the second retaining member. The first circular or substantially circular through-hole is disposed on the first retaining member and has a diameter larger or substantially larger than an outer diameter of the stem to allow the stem of the port body to pass through and to move relative to the first retaining member. The one or more second anti-rotation members are disposed at the first retaining member or the second retaining member and coupled with the one or more first anti-rotation members to restrict the port body from rotating relative to the retainer. As such, while restricted from rotating relative to the retainer, the port body is movable translationally relative to the retainer in a plane substantially perpendicular to the axial direction of the port body.

In an exemplary embodiment, the base is circular.

In another exemplary embodiment, the base is non-circular.

In some embodiments, the base is substantially planar.

In some such embodiments, a thickness of the base equals or substantially equals a distance between the first surface of the first retaining member and the second surface of the second retaining member.

In some embodiments, the first device includes a fluid connector, a gas connector, an electrical connector, or any combination thereof.

In some embodiments, the second device includes a corresponding fluid connector, a corresponding gas connector, a corresponding electrical connector, or any combination thereof.

In some embodiments, at least a portion of the stem is circular or substantially circular.

In some embodiments, the port body further includes a plurality of internal ribs disposed on an inner surface of the stem and distributed circumferentially. In some such embodiments, each internal rib in the plurality of internal ribs includes a surface for abutting an external wall of the first device to secure the first device with the port body.

In some embodiments, each internal rib in at least a subset of the plurality of internal ribs includes a first rib portion disposed at or adjacent the free end portion of the stem, and a second rib portion disposed between the free end portion of the stem and the base.

In some such embodiments, the second rib portion contacts with a knurled surface of the first device.

In some embodiments, the stem includes a first stem member and a second stem member removably coupled with each other.

In some such embodiments, at least one internal rib in the plurality of internal ribs is formed on each of the first stem member and the second stem member.

In some embodiments, the first stem member and the second stem member are coupled with each other by snap-fit.

In an exemplary embodiment, the first stem member includes a plurality of internal recesses formed on the first stem member, and the second stem member includes a plurality of protrusions, each snap-fitted into a corresponding internal recess in the plurality of internal recesses formed on the first stem member.

In some embodiments, the first stem member is monolithically formed with the base as a single piece.

In some embodiments, the second stem member includes an upper portion, and at least a segment of the upper portion is inserted into a groove of the first device, thereby helping to secure the first device on the port body and restrict the first device from moving axially relative to the port body.

In some embodiments, the second retaining member is a component of another device.

In some embodiments, a second circular or substantially circular through-hole is formed on the second retaining member and concentric with the first circular or substantially circular through-hole formed on the first retaining member.

In some such embodiments, one or more slots are formed at the second retaining member, each extending from the second circular or substantially circular through-hole to an edge of the second retaining member to accommodate tubing or cable.

In some embodiments, each of the one or more first anti-rotation members is formed adjacent to an outer edge of the base.

In an exemplary embodiment, each of the one or more first anti-rotation members is a pin formed on the base. Each of the one or more second anti-rotation members is a hole formed on the second retaining member to receive a corresponding pin formed on the base. A size of the hole is larger than a size of the corresponding pin.

In some embodiments, the retainer further includes a rim formed on the first surface of the first retaining member or the second surface of the second retaining member to set a boundary for translational movement of the port body.

In some embodiments, the port body is movable translationally relative to the retainer in the plane substantially perpendicular to the axial direction of the port body within a range defined by (i) a gap between the first circular or substantially circular through-hole formed on the first retaining member and the stem, (ii) a gap between each respective first anti-rotation member in the one or more first anti-rotation members and a corresponding second anti-rotation member in the one or more second anti-rotation members, (iii) a gap between the rim formed on the first surface of the first retaining member or the second surface of the second retaining member and an outer edge of the base of the port body, or (iv) a combination thereof.

In some embodiments, the rim is formed on the second surface of the second retaining member.

In an exemplary embodiment, the rim includes one or more rim segments.

In another exemplary embodiment, the rim is in a closed form shape surrounding the base.

In various embodiments, the present disclosure provides an apparatus for facilitating automated connection of a first device and a second device. The apparatus includes a port body and a retainer. The port body a base, a stem, a bore, and a tip. The a stem extends from the base, and at least a portion of the stem includes a circular or substantially circular cross-section. The bore extends from an upper end portion of the stem to a lower end portion of the base, and configured for receiving at least a portion of the first device. The tip is disposed at a free end portion of the stem and configured for guiding the second device when connecting the second device and the first device. The retainer includes a first retaining member, a second retaining member, a first circular or substantially circular through-hole, and a rim. The first retaining member has a first surface. The second retaining member is coupled with the first retaining member and has a second surface spaced apart from the first surface of the first retaining member in an axial direction of the port body. In some such embodiments, the base of the port body is disposed between the first surface of the first retaining member and the second surface of the second retaining member. The first circular or substantially circular through-hole is formed on the first retaining member and has a diameter larger than an outer diameter of the stem to allow the stem of the port body to pass through and to move relative to the first retaining member. The rim is formed on the first surface of the first retaining member or the second surface of the second retaining member to set a boundary for translational movement of the port body. The port body is movable translationally relative to the retainer in a plane substantially perpendicular to the axial direction of the port body within a range defined by (i) a gap between the first circular or substantially circular through-hole formed on the first retaining member and the stem, (ii) a gap between the rim formed on the first surface of the first retaining member or the second surface of the second retaining member and an outer edge of the base of the port body, or (iii) a combination thereof.

In various embodiments, the present disclosure provides an apparatus for facilitating automated connection of a first device and a second device. The apparatus includes a port body and a retainer. The port body includes a base, a stem, a bore, a tip, and a plurality of internal ribs. The stem extends from the base and includes a first stem member and a second stem member removably coupled with each other. The bore extends from an upper end portion of the stem to a lower end portion of the base and configured for receiving at least a portion of the first device. The tipis disposed at a free end portion of the stem and configured for guiding the second device when connecting the second device and the first device. The plurality of internal ribs is formed on the stem and distributed circumferentially for abutting an external wall of the first device to secure the first device with the port body, In some such embodiments, (i) at least one internal rib in the plurality of internal ribs is formed on each of the first stem member and the second stem member, and (ii) each internal rib in at least a subset of the plurality of internal ribs includes a first rib portion disposed at or adjacent the free end portion of the stem and a second rib portion disposed between the free end portion of the stem and the base. The retainer is coupled with the port body and configured to restrict the port body from rotating relative to the retainer but allow the port body to move translationally relative to the retainer in a plane substantially perpendicular to the axial direction of the port body.

In some embodiments, the first stem member is monolithically formed with the base as a single piece.

In various embodiments, the present disclosure provides an automation-compatible apparatus including a first coupling member and a second coupling member. The first coupling member includes a first side wall defining a first bore that receives at least a portion of a first device. The second coupling member is connected to or formed with the first coupling member at a proximal end portion of the first coupling member. The second coupling member includes a second side wall having an exterior surface defined by revolving a continuous curve about a rotational axis of the apparatus to facilitate operation by a robotic arm. In some such embodiments, the revolving exterior surface includes a first revolving segment proximal to the first coupling member and a second revolving segment distal to the first coupling member. Each of the first and second revolving segments of the revolving exterior surface has a first side and a second side that is narrower than the first side, and the second sides of the first and second revolving segments of the revolving exterior surface face each other.

In some embodiments, the first device includes a fluid connector, a gas connector, an electrical connector, or any combination thereof.

In some embodiments, the first coupling member is connected to the first device by a retainer.

In an exemplary embodiment, the retainer is a component of the first device.

In some embodiments, the retainer is a clip having an open side to allow the clip to fit on the first coupling member.

In some embodiments, the first coupling member includes a first external recess and a second external recess formed on the first side wall at or adjacent a distal end portion of the first coupling member. The clip includes an upper wall, an outer side wall, a first clip protrusion, and a second clip protrusion. The upper wall is configured for abutting a surface of the distal end portion of the first coupling member and a surface of the first device to restrict the first device from moving relative to the first coupling member in the rotational axis of the apparatus. In some embodiments, the upper wall includes an outer curved edge. The outer side wall extends downward from at least a portion of the outer curved edge of the upper wall and includes a first clip end and a second clip end at the open side of the clip. The first clip protrusion protrudes inward from the outer side wall at or adjacent to the first clip end for engaging with the first external recess formed on the first side wall. The second clip protrusion protrudes inward from the outer side wall at or adjacent to the second clip end for engaging with the second external recess formed on the first side wall.

In an exemplary embodiment, each of the first and second external recesses is a circumferential groove.

In some embodiments, the upper wall of the clip further includes an inner curved edge. The clip further includes an inner side wall extending upward from at least a portion of the inner curved edge of the upper wall to assist in retaining the first device.

In some embodiments, the first coupling member includes a plurality of first internal ribs formed on the first side wall and distributed circumferentially around the rotational axis of the apparatus for abutting an external side wall of the first device to restrict the first device from rotating relative to the first coupling member around the rotational axis of the apparatus.

In some embodiments, the first coupling member includes one or more external strengthening members formed on the first side wall thereof.

In some such embodiments, the one or more external strengthening members include one or more external rims, one or more external ribs, or any combination thereof.

In some embodiments, the first coupling member includes an external flange at or adjacent the proximal end portion thereof, and the second coupling member includes a shoulder at or adjacent the proximal end portion thereof to hold the external flange of the first coupling member.

In an exemplary embodiment, the external flange of the first coupling member and the shoulder of the second coupling member are connected to each other by ultrasonic welding.

In some embodiments, the proximal end portion of the first coupling member is inserted into the proximal end portion of the second coupling member.

In some such embodiments, the proximal end portion of the first coupling member includes a plurality of first external ribs formed on the first side wall and distributed circumferentially around the rotational axis of the apparatus for abutting the proximal end portion of the second coupling member to assist in securing the first coupling member with the second coupling member.

In some embodiments, the first and second revolving segments of the revolving exterior surface are substantially the same in size and shape.

In an exemplary embodiment, one of the first and second revolving segments of the revolving exterior surface is a conical or substantially conical surface, and the other of the first and second revolving segments of the revolving exterior surface is an inverted conical or substantially conical surface.

In some embodiments, the revolving exterior surface further includes a third revolving segment between the first and second revolving segments and connecting the second side of the first revolving segment with the second side of the second revolving segment.

In an exemplary embodiment, the third revolving segment of revolving exterior surface is a cylindrical or substantially cylindrical surface.

In some embodiments, the second side wall of the second coupling member defines a second bore to receive at least a portion of a second device.

In some embodiments, the second device includes a port body.

In some embodiments, the port body is a floating port body.

In some embodiments, the second coupling member includes an internal chamfer formed at a second end portion of the second coupling member to guide connection of the apparatus with the second device.

In some such embodiments, the internal chamfer is formed collectively by a plurality of second internal ribs on the second side wall and distributed circumferentially around the rotational axis of the apparatus.

In an exemplary embodiment, the second coupling member has a substantially uniform wall thickness.

In some embodiments, a tapered internal recess is formed circumferentially on the second side wall of the second coupling member at or adjacent the internal chamfer to facilitate smooth interaction between the apparatus and the second device.

In various embodiments, the present invention provides an automation-compatible apparatus including a rotational axis, a first coupling member, a second coupling member and a clip. The first coupling member and the second coupling member are connected to or formed with each other at proximal end portions thereof. The first coupling member includes a first side wall defining a first bore to receive at least a portion of the first device. The first coupling member also includes a first external recess and a second external recess formed on the first side wall at or adjacent a distal end portion of the first coupling member. The second coupling member includes a revolving exterior surface around the rotational axis of the apparatus to facilitate operation by a robotic arm. The clip has an open side to allow the clip to fit on the first coupling member. The clip includes an upper wall, an outer side wall, a first clip protrusion, and a second clip protrusion. The upper wall is configured for abutting a surface of the distal end portion of the first coupling member and a surface of the first device to restrict the first device from moving relative to the first coupling member in the rotational axis of the apparatus. The upper wall includes an outer curved edge. The outer side wall extends downward from at least a portion of the outer curved edge of the upper wall and includes a first clip end and a second clip end at the open side of the clip. The first clip protrusion protrudes inward from the outer side wall at or adjacent to the first clip end for engaging with the first external recess formed on the first side wall. The second clip protrusion protrudes inward from the outer side wall at or adjacent to the second clip end for engaging with the second external recess formed on the first side wall. In some embodiments, the first coupling member includes a plurality of first internal ribs formed on the first side wall and distributed circumferentially around the rotational axis of the apparatus for abutting an external side wall of the first device to restrict the first device from rotating relative to the first coupling member around the rotational axis of the apparatus.

In various embodiments, the present disclosure provides an automation-compatible apparatus including a rotational axis, a first coupling member, and a second coupling member. The first coupling member is configured for connecting the first device to the apparatus. The second coupling member is connected to or formed with the first coupling member. The second coupling member includes a bore, a revolving exterior surface around the rotational axis of the apparatus, an internal chamfer, and a tapered internal recess. The bore is configured to receive at least a portion of a device. The revolving exterior surface is configured to facilitate operation by a robotic arm. The internal chamfer is formed at or adjacent an end portion distal to the first coupling member and configured for guiding connection of the apparatus with the device. The tapered internal recess is formed circumferentially on an interior surface of the second coupling member at or adjacent the internal chamfer to facilitate smooth interaction between the apparatus and the device.

The foregoing descriptions of specific exemplary embodiments of the present invention have been presented for purposes of illustration and description. They are not intended to be exhaustive or to limit the invention to the precise forms disclosed, and obviously many modifications and variations are possible in light of the above teachings. The exemplary embodiments were chosen and described in order to explain certain principles of the invention and their practical application, to thereby enable others skilled in the art to make and utilize various exemplary embodiments of the present invention, as well as various alternatives and modifications thereof. It is intended that the scope of the invention be defined by the Claims appended hereto and their equivalents.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that includes a computer program mechanism embedded in a non-transitory computer-readable storage medium. For instance, the computer program product could contain instructions for operating the user interfaces described with respect to FIG. 2. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

1-53. (canceled)

54. A device for coupling a first part with a second part, the device comprising:
a robotic EOAT comprising:
a support;
a first jaw and a second jaw connected to the support and operable between an open position and a closed position for gripping and releasing the first part, wherein when in the closed position, the first and second jaws collectively define a first axis; and
a plurality of pins, wherein (i) each of the plurality of pins has an outer flange at a free end portion thereof, (ii) at least one pin in the plurality of pins is disposed at the first jaw, (iii) at least one pin in the plurality of pins is disposed at the second jaw, and (iv) when the first and second jaws are in the closed position, pins in the plurality of pins are positioned around the first axis defined by the first and second jaws at a common radius; and
a sleeve comprising:
a rotational axis;
a first wall substantially perpendicular to the rotational axis;
a plurality of keyhole shaped slots formed on the first wall and corresponding to the plurality of pins of the robotic EOAT, wherein each respective slot in the plurality of slots comprises (i) a first slot segment larger than the outer flange of a corresponding pin in the plurality of pins and a second slot segment smaller than the outer flange of a corresponding pin, thereby allowing the corresponding pin to insert into the respective slot through the first slot segment and move to the second slot segment to abut the first wall by rotating the sleeve around the rotational axis; and
a cylindrical section connected to or formed with the first wall, wherein the cylindrical section is hollow and comprises an inner chamfer formed at a free end portion of the cylindrical section to help align the second part with the first part.

55. The device of claim 54, wherein the first part is a DCap.

56. The device of claim 54, wherein the second part is a port.

57. The device of claim 56, wherein the port is the port of any one of claims 1-20.

58. The device of claim 54, wherein each of the plurality of pins is a spring loaded pin.

59. The device of claim 54, wherein each of the plurality of slots is keyhole shaped.

60. The device of claim 54, wherein:
the first wall comprises a first wall segment corresponding to the first jaw and a second wall segment corresponding to the second jaw; and
a recess is formed between the first wall segment and the second wall segment.

61-158. (canceled)
